SEQUENCE LISTING (36 sequences)

SEQ ID NO: 1 - DNA, 32908 bp, Sus scrofa
  misc_feature (21960)..(22059): n is a, c, g, or t
tcttcatcctattagagacactgctatacagcagaaattgacacaacattgtaaatcaactatactttaataaaataaaaaaaagaaatacaagtgctttctacagacaatctgcacaagttatttgttagacatatttgattatagaattaatattaaaaggggttataacaatcaagcattgataatttaattatgtttgcctattttactttagttttttgacataactgtgtaactattgcgatttttttattcctaatgtaattagttcaaaacaaagtgcagaaatttaaaatattcaattcaacaacagtatataagtcaatattccccccttaaatttttacaaatctttagggagtgtttctcaatttctcaatttctttggttgtttcatgtcccatatggaagaaaacatgggtgtgaaagggaagcttactcttttgattacttcccttttctggttgactccacctccattatgaagcctttctgtatttttgtggaagtgaaatgatttttagaattcttagtggttctcttcttcaggagaacatttctaggtaataatacaagaagatttaaatggcataaaaccttggaatggacaaactcagaatggtgctacatgaaaactctggatctgcaggtaaaatcttctcatttattctatatttaccttttaatagagtgtagcaatattccgacagtcaatcaatctgatttaatagtgattggcatctggagaagaagtaacagggaaaaggcaataagcttataaggggaacttttatcttccatagaatcaaaattgaagacgtgactagaagaaggattagatttggcatcagttttgtaaaattgctgaggtgaaattaagtaagggatgaaaattaactaaattgtgttgagtatgaaactagtagttgttagaaaagatagaacatgaaggaatgaatattgattgaaagttgatgacctagaggacatttagactaacacctctgagtgtcaaagtctaatttatgatttacatcgatgcgttaaactcatttaacattcttacttttttcccctcaagcatttaagctgaagtataacatttcacatgaaagcctggattataaatgcacagttcagtgacctatctcagaggagtgactgccatagcattttttttgtctttttgccttcagagccacagcaacgcgggatccgaagccgcgtctgcgacccacaccacagctcacggcaatgccggatctttaacccactgagcgaggccggggatcgaacccgcagtctcatggttcctagtaggattcgttaaccactgcgccacgacgggaactcctaccatagcatttttacttttaagttactgttggtttagagtaagaaggagaaatgagagtgatggagcgtttgctatatttggagacaaggtcctatattggaggttctcaaatataaattttgtcgctttttcctccaatgtattgttcaactactatttagcaggccactgtgccaggtactggtgaaactggtgaacatgatagatgtaattcattccctcatggaactttccatctaacaatgtggatcaggtaggcttggagatgagaatgccagtggttgactatgactctgtggctgaagggagagctactcacttcgtagtttcatcaatgtctttttggttttccaggttttaagccctgctcttgcaattcttttcccttctccaactttcttctaatttctcacccctaggatgcctataaacatgagtattttcaaagctacttcactgaggttatatgatcctcgtgtgaatttttcctgcctgccttgccatttagaaggaagtgtttcctggaatttccattgtggcttggtggttaaagaccctgcattgtctctgtgaggatgtgggttcaatctctggcctcattcagtgagtgggttaaggatctggtgtcgctgcaagctgtggctaagatcccacattgccatggctgtggtgtagactggcacctggagctctgatttgaccacaatcctaggaacttcagatgttgccataaaaagaaaaaaaaagttaggaagggttttctgtcttgttttgacctttgttaatctcaaacctttggaaccatctctcctccaaaacctcctttgggtaagactgtatgtttgccctctctcttcttttcgcagactttagaagatgttctgcccatttaagttccttcacttttgctgtagtcgctgttctcagtgcctgcttggtcactagttctcttggtgagtactttgacaaatttacttgtaacctagcccactgtgacaagaaacactgaaaagcaaataattctcctgaagtctagatagcatctaaaaacatgcttcatggtttcaaaggatcagatattaaaaaccccaaataggtacagaaccatgtggctctctccccccaaacaaataaaacgttagcatggttttcaaaaaaataaaataaccttcacaggaaaaatggattttacttaagatttgaaataatatctaactaaaaaatagggaataatgcagaagaggagaaacctcagaattgttgggatgaaggaatttttagtaacactaaaaattcaagtgccaaaatttgtctaaaattgtattcagggaagccagatatatatcagtgaaatcgccagttcctatattagctaaaataatcacaaggctgtagcagagacagttcagagagaggtggagatgagattttttttttttaagtataattgatttacaatgttgtggcaatttctgttgtatagcaagagatagaattattttatggtggaagataatagaaaaatatatccatatcaatttccatttgagtagataaatttcaattagagttcaactagcaattagtagttttgcatacatggtgaaatatattcatggtattttgcatatatgtgtgaaataggtactaaattcctcataactgttctttttagtctcaccatcagcctctactgatcttaggattttggagaaacatacatagttcatccctataaaatgccataaaatctcatttttacattaaaccatccaagagattatataaattgaccttataaagaatatcagccataaaataaaggtatcatagtatgggattatttagctttattggttctatgtcactgcttaatttgaaacctgtgatattgctgtttgtttttgaactcctatgaaataacattctcccattgtaccatggatgggtccagaaacatttctcaaatctggctttgaaaaataaataagtaatctaaagaataataattctctacttgctctttgaatcttgaccaattgctgcatttacctattgttacaggaggaaaagacaaggagctgaggctaacgggtggtgaaaacaagtgctctggaagagtggaggtgaaagtgcaggaggagtggggaactgtgtgtaataatggctgggacatggatgtggtctctgttgtttgtaggcagctgggatgtccaactgctatcaaagccactggatgggctaattttagtgcaggttctggacgcatttggatggatcatgtttcttgtcgagggaatgagtcagctctctgggactgcaaacatgatggatggggaaagcataactgtactcaccaacaggatgctggagtaacctgctcaggtaagacatacacaaataagtcaagcctatacatgaaatgctttgtgggaaaaaatgtatagatgagttaaaaacaaaaaggaaccagttttctataagtcatctagtccatgtataaaattacccaatccattactaaaagaccacttctggtattttacacatgacaaagcccatattaaaaaaaaaaaattcagaagagattctgaatgctataataaatgagcaagtgactagcttcaattttatattaggtcattctaccttctacttctacatgaaaatatcataatgtctaagttaattccttgtcccctttcccaataaagcactgctttcatgcactggcctatgaatcatgaactttttgccctttaactgatgatcaacttaccaaatcaagaaataaatattcttagcactgatccttttttgttgttgttggaggaagaatgttttgcaaagtagaattgcttttttctgtttaacagtgctattcatttcatttacatggtcgttttaatttataaaacatttcataagtttcacctcatatgcccttacaataactcaggaagttatatgttagacctttctgctgacaaatcccagagtcatgtttctgacccagttcagattccttggcttcccatttctctttgctcatgtcattgacctttatgcagccctcttacctcccacctttctattacagaccatctcctccataggactggtgttagaaagtactaatctctacccaggcattgtggtgcaatgtgggcagcacaggctggtatctagaaaaatgctgaagtgaattccagctcagctgctcgttaatactattgttttaagtaagctgttcaatcctttgaaattcactttctgagcactcagtgatataataaatgtagagttactggtacactgtctggtatgtaataggtgttaaaaattaaccttagtttcctcatgggtcactgcttctcattacctagacaactcatttctctttcttcctctttctctttctccattctcctcctccttcttcctcttcttcttgtcttttattgttattcattttgctgagaaagttaagaaataacaactctaacctctacatcgaccacctagagcaaagttaaaaataataataaaccttgccagactcttactataattgttgctgtctatagagttgactgtttaagttaagacatcagtatagtatttttaatttttgtgttttttttttcatacttttacatgaggatcctttatataaggatgagttaaacaaacttgatttttgaagtttatacccctgaggctcaactgcataataatagaaagggatccatagcctctcaaggacttaactagtttcatgagttttcagaatctgaatttctgagattctccaccccaattaaagctcaagcctcagaacatatatccttctcttggtaaattctattcttatcacatgcgtaataataaaaaagagagatgttggagacagatttttttcctcacattctgtctctactgttttctaggtgtttgattctgtgttatttaacctcagtttgcttatctgtgaagtagggattatggtaataacatataatgcttaatgttgtaaagactaaagaagatagcatatgtaacacatttggaacagggaatgcatattttgattgtgagctcttattattattaccaatcagccataataaaaatcttgttaagtggaggtctttggatttcagagcttttaaaatctaattactttttcaaaaaagagcttcttagtgttttttttttttaaccacaaagtgtttctattttttaggtgtcccaaaatttcattccaaatatctttttctcagatattttagtcctcatagaacacctagggatagtgtatagagaaaattttctttattaaaaagctgttctttgctaaaaattgtagcaggtacttttgggaggggggaaaactttgattcagaaactgctaagacatggagtgttttgactaatttttcctcaatttttaatgttttttataccatagggtacttttgcaaactattatgcatacttatatatttttacttttttcctgtcttttaacttccaaattcaacttcagacaattattcatgcactaaactgttgtagtaagaaagattaaaattaaaaaattaaccattcaacaaatgactggtttgccatttttactactttgttgtatgaacaatttttttttctacaaatgaatactttgagtctgatttatccattcctacataaaagtttttactatatcttagtattggaaggaaacaaaacaaaacacaatgtaaattttaatctataaattttgggggggggtaaatatacatagatgaaagtcttaaccattaattagagtcaaaagattaaaattctccaatatgtgaacttaggctgcatccaaaatgaagcatcatttttaaggacagcatcaaaagtgaccagaggaattttactttctttctttttttttttttttttgaattttagtttctaaactcacttctgaataaatacaacttctaaattctcgtcttttctctactctagatggatctgatttagagatgaggctggtgaatggaggaaaccggtgcttaggaagaatagaagtcaaatttcaaggacggtggggaacagtgtgtgatgataacttcaacataaatcatgcttctgtggtttgtaaacaacttgaatgtggaagtgctgtcagtttctctggttcagctaattttggagaaggttctggaccaatctggtttgatgatcttgtatgcaatggaaatgagtcagctctctggaactgcaaacatgaaggatggggaaagcacaattgcgatcatgctgaggatgctggagtgatttgcttaagtaaggactgacctgggtttgttctgttctccatgagagggcaaaaaaaggggagtaaaagtcttaaaagctcaaactgttaaaaacataatgatgattgcttcttttatcatcttattattatctaatttcaggtcgaaattctagtacctgtgcagttttttaccttaactgaaattaagataaataggatagggaggaaggatgagcagtgacatttaggtccaagtcatgaggttagaaggaaatgttcagagaatagcccattccctcagccctcaaagaaagaaagaaagaaaaagaaaaaaaaaaagaaagcttaactagaaaattttgttctctggatgttttagaggcaaaccatcccttttatcattccttacctacaaagcccttctctttaatcacattgacccaccctttcctaaactattagttcaaattcacataattgaatgcttttaaaacttggtttcctcttataattatatttatgttgtaaggaggcactgtgtcttgtctagagactttcatgttctatgcttgattatgggacagggacatggctttgtctgctccaggatgtcactctccttttttcacttgagctcctagtttgaagaagacctagtaagtcttgaactccagggagtctttaggaaactatccctagagcaaaactgtccctgaattcacccagtgtcttttttttttttttcaaatgaaggaactttagttcaaactaaatttaaaataagggaattctaattcagaatactgggaaatccaggagattacaattggcttcatgtgtgattggattcagcacttcaccaatgtcatcagggttctggttctttttttatttcttgaattggcttttttttttttttccttgttgaacaatatgactatctatactttgaaccacaaagaaagtgattcctacagaaaagacagaatgtgttagctgaaggaagggaatgggacttggggtagaaaaaaacaccttccgtattccttaacctatcaaaaatttctaggtacccctaactaaaatcctaattcaagcatattggaggaacttgacaaatccaggaataatattatccgttatcaaatacatgcacatcatttacatttctccatgtctctgctcatgcagttcccggccctaactctaccaaagtattactctccatctccctctttttttttttaatgatttttattttttctgttatgactggtttacagtgttctgtcaattttctactgtacagcaaagtgacccagtcacacattcatatatacattctttttctcacattatcctccatcaggctccatcacaagtgactagacatagttcccagagctatgcagcaggatctcattgctgctccattccaaaggcaacagttcacatctattaaccccagattcccagtccaccccactcccttcccctccctcttggcaaccacaagtctgttctccaagttcatgagtttattttctgtggaaagttttatttgtgcagtatgttagattccagatataagtgctatcatatggtatttgtccttctctttctgactgacttcacaaagtatgagagtctctagttccatccatgttactgcaaatggcattattaatctccatctttttttgttcatgtatatgttacccagattccttgacttttctacatcatcaagatattgttgatcacttctttgtagtgatttctgcccttctctgatgtcctgtgacactagtctggattattcatttacctgaaaccacatgtctcttataatgtgtatcccaaattaaatatgtctattgtaatgtgtatcccaaattaaatatttatctttctaaaaaaaaaaatttctaggcccccaatcagcatgtttcttctcagtgtgttttatacatgctgcagaatcataatagacagcataatagacagcataacaaaaactaaaaatgccaggggaaaaaagcaatttactgattacaacatattactcagaatcaagttctgttctttgaggaatattgattgggggaaaatgaaaataatgatggggaggtcccttttctctttgctttgcttttaaactacggaagtagtcagaaaggggtcaggaatgtaatataaaccaggtagtcctggtaggtaacgcagccggaggcaaaagtgagtgttgagtattgaggcaaactggagggcatggataccacctagacagatgcaaatatatatttaacagggaaaaaagaaccaaacaatttcaacaaaaaaccaaacaattccaacaaaattggtccaataagcaaacctctagataaatttcagtccctggatgttttgttaggaactcttcctacaatgcgtgctttccattctgaaaagtcctatctacttgcctgatccacttctccttccatcctaaacgattttcagtggtagtatattactgttgtctctgtctctacttatatatcttccccttttcactcactcctctcaggtacagctcttcagtttgcccttattcttgtttccttgtcaatgacttgttttgtgtccctcttacagatggagcagacctgaaactgagagtggtagatggagtcactgaatgttcaggaagattggaagtgaaattccaaggagaatggggaacaatctgtgatgatggctgggatagtgatgatgccgctgtggcatgtaagcaactgggatgtccaactgctgtcactgccattggtcgagttaacgccagtgagggaactggacacatttggcttgacagtgtttcttgccatggacacgagtctgctctctggcagtgtagacaccatgaatggggaaagcattattgcaatcataatgaagatgctggtgtgacatgttctggtaagtgaaaacaaaacaccggaaggacctgtgttcttcaggattaggaatggatatgagataggagaaaaattgtatctaatattttctttgttgggaattcttttacagttgtgacaaatctttaacatattcttcatttgagtagtttggagggttgtctgactgttttctataataaatgtcccaagtgctatgaggtaccacatttcaaattctaattctacctgaagctccaaaaagacaaaatgttataggtcttttctttatatctaatttgcttatggtttttagccattgacaattttttttttcttaactcttgaaactataatcctatttctaaccaaattcatgttctatactggctcttcaaaaacccaggagatgggaaagccagaatctccagtgtttcagcttctgggaaggagcaagtttttaaatgtgggagctaaattccacatgtatctatggcctaagtgtatgtttattttgcagatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaagccaaaattacctgctcaggtaagaatttcaatcaatgtgttaggaaattgcattctactttcttttacatgtagctgtccagttttcccagcaccacttgttgaagagactgtcttttcttcatcatatagtcctacatcctttgtcataaattaattgaccataggtgtgtgggtttatatctgggctctctattctgttcctttgatctatgtgtctgtttttatgccagcaccatgctgttttgattactatagctttgtagtatcatctgaagtcaggaaacatgattcctccagctttgttcttctttctcaagattgttttgtctattcagagttttatgttcctatgcagattttatttttatttttattttatttttattttttttattttcccactgtacggcaagggggtcaggttatccttacatgtatacattacaattacagtttttcccccaccctttcttctgttgcaacatgagtatctagacaaagttctcaatgctattcagcaggatctccttgtaaatctattctaagttgtgtctgataagcccaagctcccgatccctcccactccctccccctcccatcaggcagccacaagtctcttctccaagtccatgattttcttttctgaggagatgttcatttgtgctggatattagattccagttataagggatatcatatggtatttgtctttgtctttctggctcatttcactcaggatgagattctctagttccatccatgttgctgcaaatggcattatgtcattcttttttatggctgagtagtattccattgtgtatatataccacctcttctgaatccaatcctctgtcgatggacatttgggttgtttccatgtcctggctattgtgaatagtgctgcaatgaacatgcgggtgcacgtgtctcttttaagtagagctttgtccggatagatgcccaagagtgggattgcagggtcatatggaagttctatgtatagatttctaaggtatctccaaactgtcctccatagtggctgtaccagtttacattcccagcagcagtgcaggagggttcccttttctccacagcccctccagcacttgttatttgtggatttattaatgatggccattctgactggtgtgaggtggtatctcatggtagttttgatttgcatttctcttataatcagcgatgttgagcattttttcatgtgtttgctggccatctgtgtatcttctttggagaaatgtctattcaggtcttttgcccatttttccattgattgattgttttttttgctgttgagttgtataagttgcttatatattctagagattaagcccttgtcagttgcacctatgcagattttaaaactattttctctagttctatgaaaaataccattggtaatttgatagggattgccctgaatctgtagattgccttggatagtattgccattttaacaatactgaatcttccaattcgagagcacagtgtatctttctttctgtgtcatcttcagttcttctcatctgcatcttatagttttagaagtacaggtcttttgcctcctaaggtgggttttttcctaggcattttattcttttcaatgtgatagtgaatgaaattgtttccttaattctttctctctcttttttaatggcttcacctgcagcatatggaagtccccaggctagggatcaaatcacagctgcagctatgtccatgccactgccttggcaacagcagatctgagccacatctgccacttacactgtagcttacaataatgctgaatccttaacccactgctagaacctgaatcctcacagaaacaatgtcggggtccttacctctctgagccacaatgggaaatcttcatttttctttctgataatttgttgttagtgtatagaaatgaaacaggtttcagcatattaattcttatcctgaagttttacccaattcattgataaactctagtagctttttggtggtgtctttaggattttctatgtatagattcatgttacctgcaaacagtgccattattacttccttttttccaaattggattccttttatttctttttcttctctgctgtgactaggatttccaaaatcatgttgaataaaagtagcaagaatcagcatccttgctttgttcctgaccttagaagaaacacattcagcatttaactgtcgagtatgatgttagctgtgggcttatcatatatggcatttattattttgaggtatattccctctatacccactttgttgagaactttttatcatgaatggatgttaaactttgtctaaagctttttctgcatctagataaccctattatttttcttttctaatttgttcatgtggtgtatcacactgatttatttgcagatgtgcatccattcatgtatcccacttgatcgtggtgtgtaatctttttagtgtattagtgaatttggttgctagtattttgtttgaggatttttgcatatacattcatcagcggtattggattttaaatcttttgtatgtgtcttgttttggtatcagggtatcctctagggtatcctcctagaatgagttcagaagggtacatttctttggggaatatatttggtagaattcacttttgaagctgtctggtcctgttcttttgtttgtcgggaagttctttttaaattattattattactgattcaatttcattactggtaattggaccatttatattttcttttttttcctggttcaatcttgggagattgtatgttttaaaaatttgtccagttcttctaggttgttcattttattggaatgtaattgtttgtttatctttttttttgcattttctagggccgcacccatggcatatggaagttcccaggctaggggtctaatcggaactgtagccactggcctaccccagagccacagcaacgtgggatctgagccgcatcttcgacctataccacagctcacaacaatgcgggatccttaacccactgagcaaggccagggattgaacctgcaacctcatggttcctagttggattagttaaccactgagccacgacgggaactccaatggtatgtaattgtttatagtgatctcttatgagtctttatttttctgtagtaatcataacttctcttatttcattttgatcttattgacttgagccctctgtttttttcttagtgactctagctaaaggtttatcaattttgttcatttttttcaaggatctggctcttaatttcattcaacttttctatttattttagtctctatttcatttacttctgttcagatttttatgatttctttctttctactaagttcagttttggtttgttcttttctatttcctttaagtgtaaggttatgttgtttatttgagatttttgtttcttgaggaaacaggcttgcatatttgtaaacttccctcttagaatagtttttcttaagttccatagttttttttttttattttgtggtttttatttttccattatagttcatttacagtgttctgccaattcctactatatagcaaagtgacccagtcatatatatatgtatatatgtatatatacacatacatatacacattatcctccatcatgttccatcacaagtgactggatacagttccctgtgctatatagcaggatctcattgcttatccactccaaatgtaatagtttgcatctattaaccccagatgtcccatagatttggaattgtgtttttgttttcattcgtattcaggttttttttaatttcctctttgatttcttcagtaatccatttgttgcttagtaatatattgtttagcctctgcgtgtttgtggtttgttgcaattttcttcttgtagttgatttctagtctctttgtgttgtagttggaaaagatgtatgatatgatttcaactttcctaaatttaccaaggcttgttttgtggcctagcatgtgatatatcctgaagaatgttccatgtgcacatgaaaaaaatgaatattctgctgctttcaaatggaatgctctctctatttcaattatgtccatctctaatgttttgggaacatgttcttttgctacctcattttgcctaatttgctgttttgggttctaaatatctggtaggttggttacattttccaaccttggacaaataaccttttgttgaaacatcctgtgcttcccagcagcacactcctctctggtcaccagagctatatgttccaggggtgcccccctatgctgactttgtgagaacttcttttgcagttggctgactactgtaggtggtcttgtaggcatggctggcccccagtctggttgtttgcaagaagctgccttgtacaaaggctgccagtcacttgttggtgggactgggtcatggggtggctggctatagagaccagggttgtctcaggggtagtgctgtctcatttgtgggtttagccacgttttgcagtgggtgattgtggttccagggttcctagatctagtgtcagcttgtgggtactggggtccccagctgcagggcctaggagcttcagagctagagctaacctcctggtgggtagactgtgtcctgacaaggcaggttgtagtgttacagtgatcctggggctagtatctatccactggggggtaagacttgtcccagggctagcaccagctctctggtgggtagatctaggtcctggaggttctggctgcagggccagggatccaggagctggtgttgactggttggtggacagggccaaggcccagagtgtccccaggctagatctacttcagtgatgggtggatctaggtcctgtatttctggctacagggctctgggatcccagagttggtatgtcagtcaactgacatacagggctggaggcagagagtcctgaggctggtgcctgcccactggtgggtggagctgggattcagggtctctgactgaagtgccctggggatccctgggctagtgctggcccactggtgtgtgtttggttgggtcctggccattctggtagacagggccatattcccatattccagggtggctgtaggctcagggaatctcaaggcaacctactgctggttagaggagtgtgtggggaggtgctatgtccctgtccagtttgttgcttggcatgaagcatcccagtactggtgccaacaggctaattagtgggtctgggtcctggtgctaataagctagagggaagattcaaaaatgacatttttttaacaccagtgtccttgtggtaaaatgaactccccagaatggctaccaccagtgtctatgtccccatggtgaattctaattgctcctgtctcttgaagtggctctccaagatcaacaggtgggtctgatctaagctcctttcaaattactgcttctgccctgggtcccagaacatgtgagattttgtgtgtcctttaagagtggagtctctatttcccactgctctctggttctccccaaagtaagccctgctggctttcaaaacttctgggagcttgccttcttggtataggactcctgggctagggagtctaatgtttggcttagaccccttactgcttgggaagaatctctgcaactgtaatgaattatcttcctatttgtgggttgctgaggatatggtcttaactgttctgtgttctacccctcctatccatcttgttgtggttccttctttatatctttagttgtagaaaagtttttcttatcaacagttgctctgtaaattgtaacttgggtgtacacctagtaggaggtgagctcagggtcttcctactctgccatcttggccatgtcctctaaacattttggtgtatttcactgcaacctttttaaaaatctcaaaagtgagctgtgattggctagtcttgtggataatctctagcatttgatgctaatcatatttatacaaatactttgttgaaaagtgatgcctttttaactattattaaaaaacgtattgacataactattgctattatactgaaaagaaagaccttagagaaaatagcataagagcaaaaccattaaacatggagacatctagtcatagggtggaaattttatgtggtgcatatcccctaaccagtggctttacaccaggcacatcctaactaagatctgctcccaagtgtcttccctgatgctttaaattgtgttacatggaaactatcctttgatgaagaaatgcaaccttttaaaatacaacattgaaacttttgtgctttaattttgcttttcaacattttttctttttaaaagaagaaatttatttgtttttttaaattttaatggccacggcatatggaagttctcaggccagggatagaattcaagccacaggtgcgacccatgccacaactgctgcaacaccagatcctttaacccactgcaccaggccagggattgaagccttgccttactgacaatctgagccacttcagtcagataaagaaatttcttcattaagcagagtattcacatggtttaaacttcaaaatattaaagtgtaaactctttccccaccactgtccccagctcaccaactctacttaccacagacaactgatgtggttagggtatttaaatagtaaatccaagaaaatataaacaaatccgtatatataggtttcaccccattttattatcctaatgttgcatatcatataaactatactgtcccttgggtattcacttagtaaaatattttgatcataatttcctatcagtatttaaagagctttctgaaattatttctgtataacatttcttttctcatcatctattatgtgcatttatttatattttaacttcttttattagatgaaattatcttctgcttcagcttttttttttttttaagaacacacagttgggttttttaaggttaataccacctttgttttctaagtcattaaatttgtttttctattaattcacttctgattctttgaagtttgatttctttttagcttttaacttcttgagttgtatgcttaattaattttgattctttcctatttattaatatacatatttgaagctataggttttccactgagtataccagtagctatatcgtataattgatgaactgatcctctgtgagtctgggacataaacgtcctatgactgttatgtggtagctgtgaattgctctttttagattataaagttctcatcttttatagttgaacaatttttgtcctgaatcaaatttgttggatattaatatcacatctattgctttatttattttctattctcacttttaacctctgtgaataatttcactctaggtgcctcacttttttcataatagaattgggatttatttttaaaaggactctgattaagtaattttctttttctgatatgggagatatatttgaccttaacttagtcacattatgcattgttctcttgtcatgttatgtatacataacatttattgtcattatggtacaactaaaaacatatttcactctgtgacctttatggggactcagcatttgtttaggaatgtggaagtatatttgtatatctgataatttccttccaaatttaaaaaggtttgtatattttcatattaacatatttcatattaattagcatgaatttcagctgcattaaaaggaaaaccacctgagtggtaaagaaaaagtttttttttctcttttttttttttttttttttaatggccacatctgtggcatgtgaagttcccaggctaggggkcgaataggagctacagctgccagcttgcaccacagccacaacaatgccagagccaagcctcatctgcgacctataccacaactcatggcaatgctggttccttaaccccctgagtgaggcctggggtcaaacccacatcctcatggatactaaccggctttgttaccgctgagccatgagggaaactccctttttctcattgaaaataagtcaaatagataagcagcttaaggctgtttgggtgattctgtggtccagtaattatcaaatcctactggacaagaatagagaatgtgcaaatgagggaacgtgttggtgagatcaggctctgcccactgagctatcctctgtcatgggccctgtgctgttctcagagctgtacttcctagggcattgttctcatttcaattctgagttcagtgtggagagtatacgtgtgtgggggctgcacgcttttcacaacccactttctgctgatactgatttagggatccttggattgctttacagttgagtcatcattaactagtgtcacttgccttcaaagtcagcaaaataattgtctccaaactagtaggcttctagtgtatttgctttaatccaatgccatgtgaaagtaacatggtcaaagaataagttatataccttgacctaccctgtgaccaggctcttcctcttaatttattgaccactgccttaaggtcatttgaaaccatgggtttgggaggaaggcaaggcctaaatcccgtctttgttggaaggctcactgtccttgtctttagagcatcatttttttttaaactggggtacagtttatttacagtgttgtgtcaatttctgctgtacagcatagtgacccagtcatacacatacatacattctttttctcatactatcttcaattttattttgtgctaagtctgccattttatcatcacctcagtttgaaggacaggatatttagagtttgttttttttttccccccaatcctgcaatttctaaattataagactctcaattagccgtatataacagctgcaggcacaggatgtctccctcacaaaattggtatttttccttccatttcttcttgcagtttggctatttcttgtctgagttcatctctctttttaagtgttaaaaagggcaaggaggattcatgctatgtcaacattatgattttttcttttctatacttgataagagtatacttttcccaaatgtcatccaacttttcagcatcagtttggacatggttttcttttcaaggtggtatttctctaatgtcacttgaataacaagactcgttagttctccaggctacaatatcctagtctgagtatattctgcatgttaattctattcagccacatccataatttaggttttattcctggaacacctcactttttttttttttttggtctttttatagccataaccatggcatatggaggttcccaggctaggggtctaatctgagctttagccactggcccatgccacagccacagccatgccacatctgagccacatctgtgaccttttccacagctcacagaaacaccagatccctaacccactgagtgaggccaggggtcaaacctgtaacctcatggttcctagtcagattcgtttcctctgtaccacgatgggaattcctaatacctcacttatgataacacattctgaattatttaggattctattatactgcatgtaatagaaatcccaaatagcaaaatttgcaacttaaggcaggttcctgtctttacaaaatcatgttttcctttgctatatgtgcactttgctttcctctgtgaattcccttttttgttatatttctatagcttttggaaacacttttacttatttgggggggcctagatttttaaccctctccttgtttttctagaaatagagtttataattttatttcttcatttacttgatactttcaagagatttccaggaaaaaaattatggaaatactgtctctgtgcctgccaagttcaaactaagaattgtataatctgttttaattcttaagcatttatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcattaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaaccaccagaggtatttatttgtttttgccttttttcactgactgttttttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgttgcagcttcaatttgacccctagcccgggaatttccataggccacacgtgcagcactaaggaaggaaaaaaaaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgggtaagttctgcacatcacttcgggttacaatgatttaagaaacaactaaggtggggcaaagggtagtgaggcatatccatcagagcaaattccttgaaatacggactcagaggaaaccattgtgagattgaggttcccagaggtgtggatttaatgaattagtgttacctcatgtacaaggtagtatactaccagaaagataaaaattcagaagcgagtttgcagcaaaactcatagggagaacttcttttataaataatatgaagctggatatttagtgcaccacctgatgaccactttattaataaataaagagttcctgttgtggcgcagcggaaatgaatccgacaaataatcatgagtttgcgggtttgatccctgacctcgctcagtgggttggggatctggtgttgccatgagctgtggtgtaggtcgcagatgctgcttggatcccgctttgctgtggctgtggtatagtcttgtggctacagctccgatttgaccgctagcctgggaacctccatatgctgcgggggtggccctcaaaagcaaaataaataaataagtaaataaataagtagtttaaaaaggacaagaagaaatatatttggtattatattctacagagacaaagataatcaccatgcccgattgatttttcaaggcatataaatgagacgtcatgggagcaaaaatggtcataatacaatgcccttgttttgtgtacatggtaagattttagaaagcattgtgaagtagaaaagtgtactcagttataatatattggagaaaacagtactatgagaagtaaaaaaatctacatgccggaatttatttttttaatgtctctttagagtcgcacatgcggcatgtggaggttcccaggctaggggtcgaatcagagctatagccactggcttatggcacagccacaacaacgctagatctgagccacatcagagacctatactatagctcatggcaatgccagatccttaacctactgagccaagccatgggtcaaatccaggtcctcatggatcctaggcaaattcatttctgctgagccacgaagggaactcctcagaagtgattttgatgttactttcttttcatgacaaatctggtaaagtacatacacatagaaactgaagtgtcagaaagggaaatatttcattttaaggtaatgtatacaaaacagtggttttaccatctgagtatcttgctaaattttaactatcaaggacaattgccaaaaaaaaaaaaaaaagagagagagagagaacagaatagggttatgaagctaaaatcacagggttatgaagctaaaatcacagtaatttagggagaaaaaaatccaaagcatgtaattgataaaaggctctgagcctttgtttgagatttagaattcaacttggaaataccggtggtattttaaagcagtccataagtataaaatccaaggctaaaaagccagaaggtatttgtagaacaaatatattttaataagctctaccaagtcatccagaagctactaaagaattactggtcactgacatagtgtacctgttttcaaggccattcttacatcagaataaagggagagcaccctctgaatcttcagaaaagatgtgaaagtgctaattctctatttcatcccagagttcatgtctctcagactgatcagtgaaaacagcagagagacctgtgcagggcgcctggaagttttttacaacggagcttggggcagcgttggcaggaatagcatgtctccagccacagtgggggtggtatgcaggcagctgggctgtgcagacagaggggacatcagccctgcatcttcagacaagacagtgtccaggcacatgtgggtggacaatgttcagtgtcctaaaggacctgacaccctatggcagtgcccatcatctccatggaagaagagactggccagcccctcagaggagacatggatcacatgtgccagtgagtatccattctttagcgccactgttatcttctgatctacctaagcagaagtgttataacctttagataatccctattctacctggatgatgagattcattctctttaatttggtgtgcaggtattcaggatcagtgatcattttcccaaagaccatcatgctctgatggtcttctcaaaagttctaatcagttgcttcctccgtgaacagttgaggagcagagaatatgtaattcagaatttgactattgaatcatcccatttttctttcacatagtcttttgttgcactgagtataaggagagaagcagtcagaaagatcaatcctgaattatttctccattctacatctgttttaaatttcaaaaaaaattgttataggtgatttacaatgtctgtcaatttctgctctacagcaaagtgacccagttatttacatatacattctttttctcatatttttaaaccgggagatttctatccacctggcagtttgagggaatttaacattatgcatttatgttaactttattcacctgatgttttctaagtcatactgagattcttatgtccaggatggaatacacctggtttgctggaaagacatgtgctttcataaagatgaattttggaaaaaatataaaatttaaaagtcccattaaataagcaaagttttaagagatttcaaaaaaaatttcatctctctcttttcctctttgacctcttgggcacgttcatcttctcaaatatgatcttggtgtttctgacttttcagacaaaataagacttcaagaaggaaacactaattgttctggacgtgtggagatctggtacggaggttcctggggcactgtgtgtgacgactcctgggaccttgaagatgctcaggtggtgtgccgacagctgggctgtggctcagctttggaggcaggaaaagaggccgcatttggccaggggactgggcccatatggctcaatgaagtgaagtgcaaggggaatgaaacctccttgtgggattgtcctgccagatcctggggccacagtgactgtggacacaaggaggatgctgctgtgacgtgctcaggtgagggcagagagtctggattgagcttggaagctctggcagcaaagagagggtgggcggtgacctgcattgggtaaagattggaaggtccagcctaaggatctggtggtggggggagacatgatgtttcagtctgaagaatgatgaaaacctgtgttgttacgcatgggccttcaccgaggaaaggaacataacttacatgtatcctcctgcagagggaggaagaactaggggattctagttttgtgtgggaaggagcagtttacttggttcaggaggcactaaaggctcagataggaaacagagatctgttccattcttactcccagaactgattctcttctcttttctcctacagaaattgcaaagagccgagaatccctacatgccacaggtatataaaaaagtttaagaacatgggacccattgtctgcattttgtggaatccctcttattaagacattctgggtcagaagttctgaggatttgacatttacttcagctatctgttatcttacccaagagagggatggtaactaggaacccaggtcttttagctaagacattatcacctcttgtgatgtttacttgttctcaggtcgctcatcttttgttgcacttgcaatctttggggtcattctgttggcctgtctcatcgcattcctcatttggactcagaagcgaagacagaggcagcggctctcaggtctgaacaaaattacggtctctctaatgtttctatgggataagaagcctctctggataataaaacaaaaaaattacattcaagtatcagttggccagaaagagggaacctagaagaggtttaagcagtttctccgaaacagggaacaagaattcagagaagaaaaggcacattggctgtactgatgatacctgcactcgctatgtatgtttaatgggggacagtagagaattgatagtttagaaggagtatgcttatatggttctggatgaatcctgtatccccccaaacatttattttctcttactatatacttattactaatttaactcttctgtcaagccgtgtgctaggttctgaagatggttcagacttggatactcaagtgcttttgttttcatggaatttccagtttagtggaagagataaatatgtaaacaaataaattgcaatgttttattatacattcgtgtgaataaggaacaaaggaggcacagagaataaagtaattactgaaaggggaaggggagtatcagagacttctaagtttggaggcagattttgaagacagaaatcaaagtactgggtaagatgcatttcaggaaagaagaaaaatatgtacacgtgtagagaagcttaaaagagggcacatttgttgttttggaggggagtacaagttgagttaaagagagaagtttctgttaaggctgaagaatagggaagatacacgtagcgatgctctgtgttgcatgataagaagagtcggagttattaaagagtatgagataggggagtgagataggcaggcaggtccttagaaagttctgtttggaaatgggatgtcggaggggttgaaagagaaccatatattgacaaggagagcattttgaagtagttgtgatgaaagataaaatggactttatagtgagaatggctgggaaaggatagattttatacaaatctccaatgaattacagaagaatgctacctgtctttggggaagaaacagggttatccgatggcatcctgttgcgtttgagttcgtgacatcatgagggaaaggcttggcagcgtttactcggtactgtgtggtaacttatatggaaaaaaatatgagaaggaatgagtgtgtgtataactaatttacttagctgtatgcctgaaattaatacaattttataagtcaactctactccaataaaacaaacaaataaataaataattttaactacctgaacaaaaaaaaaagaatggactggagacaagtcaaaagtatggatgatgactacgttatgcttgcactgctggggaaaagcacacatagggagggaacgttttattatgacccagtccctaacctatgacctctgttatcagttttctcaggaggagagaattctgtccatcaaattcaataccgggagatgaattcttgcctgaaagcagatgaaacggatatgctaaatccctcaggtccgtgggttctttgagggcctgtagccctggggttcagatcagcagctgcagttgaggttgaggcatgctactttgcacagcagtagaaagaaatctcaactgtaataggaagcttgggatgcatatgaggaagaaaggcaagaatgaaccacaaattattcttagggaagataaaaattgcagtcatggggagacctctggctgagagggccgtgattatttctgacagagggattatggagtagaatatgatggcttggaccttttttcactaaaacaagtcagtcttctcaaaggtagtttagcttttcatatatctttctcagtttcttccattcccatttcctgccattttcctttctctaacttttatttattatattttttcctaaaagtttaaattttctatatctttatcccttcagaagccatccctagtcacaggactagttttatttcccattatgtaatgcttctttctctgtctgttgacttctatttagaaccagtgcactaaatctgcctctaggaacatacctctgctaggttgcaagaaatatcccattccccactcactctgtgaagactcaatgcttctcaatattccttacctcctgagagggacttgcctcacttctttaatccaagggactcgatttttgccaaaactaagtcaggaaaacctacataagacataggaaagacttgctgtgcttcttaaaccccactgtttgttttcctaattgtgaacagtatttttaaagttcaaagagcttctaaggcacttgaggggagatctgatttatttcccagtaattattttattcctttcagaaaattccaatgaataagatggttttaatgatgtgggactaatttttgtgtctaaatctcttcctatttctggatgaaaaaaaggagaccactctgaagtacaatgaaaaggaaaatgggaattataacctggtgaggtgagtaaaaagaatttattcatcattgctgaaaacaggtacattccttttgaaagttgggaactcctctggtattagaaaaaaaaaaaagaacgtatatacacatatatttccatgtctatgtttatgtttgtaaatccatattcagaatatgcaacaactttttataactatgacttcagtccatcttttagttacatatatattctaaacaacaactattgctaagagaagctgggtaagtaaatgtgaataaatcttctaaagatattacaggaagttcctgctgcggctcagtgggttaaggacttgatgtctttgtgaagatgagggctcgagccctggcctcactcagtgagttaaggatctagcattgctgtaagctgcagcgtaggttgcagatggggctcagatccagtgttgctgtggctgtggcctcagttgcagctctgattcaacccttaggcgaggaacttccatatgcagcaaatgtggccattaaaaaaaaacaaaaaacattataggagtcatttcataaaagagataagacgtttctatagttatatagtgcatactctggtaaagatagtataggatactataggaatatagaaagcttgcctatgaaaatttgggaagattgtggaaaagacatctcaaaatatggcatagaaaagaatcatatctttgaggaacagtaagtttttcattcaaaaccgtgtattgaacatacttatggtgacaaatggtgtcttgagtactaaaaattcagtgataaaagatgctcttgacaaagacatggctgttgaatagaaggtctcactgtcaatgtgtgggaattatggacagcctatgtggacacagggaatagatgagactctaggctggaaggctgcattgagcccagtaatgaatggtcctgtctgatatatttcatgctcatattttattttagggactattggggaggtggtgggctttggaagattaagctgaggcaagacacaatcagattgccttttataatttactttcaggaggaaaatctaactaaagaaaaaaagtgaataaggcaagaaacataagttatacatcaaaaagaaaaggtagtggagttcctgttgtggctcagtggttaatgaaccctgctaggaaccatgaggttgtgggttcgatccctggccttgctcagtgggttaaggatccagcgatgccatgagttgtggtgtaggtcgcagaccgtggcttgggtcccgcattgctgtggctatggtgttggctggcagctgcagacagctctgatta

SEQ ID NO: 2 - protein, 104 aa, Sus scrofa
HRKPRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCS

SEQ ID NO: 3 - DNA, 32 bp, Sus scrofa
aatgctatttttcagcccacaggaaacccagg

SEQ ID NO: 4 - DNA, 32 bp, Artificial Sequence
  Modified splice acceptor site
aatgctatttttcggccatggggaaacccagg

SEQ ID NO: 5 - DNA, 20 bp, Artificial Sequence
  sgSL25
tgaaaaatagcatttcggtg

SEQ ID NO: 6 - DNA, 20 bp, Sus scrofa
caccgaaatgctatttttca

SEQ ID NO: 7 - DNA, 20 bp, Artificial Sequence
  sgSL26
gaatcggctaagcccactgt

SEQ ID NO: 8 - DNA, 20 bp, Sus scrofa
gaatcggctaagcccactgt

SEQ ID NO: 9 - DNA, 20 bp, Artificial Sequence
  sgSL27
gtcctccatttactgtaatc

SEQ ID NO: 10 - DNA, 20 bp, Sus scrofa
gattacagtaaatggaggac

SEQ ID NO: 11 - DNA, 20 bp, Artificial Sequence
  sgSL28
cccatgccatgaagagggta

SEQ ID NO: 12 - DNA, 20 bp, Artificial Sequence
  sgRNA 1
aaccagcctgggtttcctgt

SEQ ID NO: 13 - DNA, 20 bp, Artificial Sequence
  sgRNA 2
caaccagcctgggtttcctg

SEQ ID NO: 14 - DNA, 20 bp, Sus scrofa
acaggaaacccaggctggtt

SEQ ID NO: 15 - DNA, 20 bp, Sus scrofa
caggaaacccaggctggttg

SEQ ID NO: 16 - DNA, 99 bp, Artificial Sequence
  HDR template
gaaggaaaatattggaatcatattctccctcaccgaaatgctatttttcggccatggggaaacccaggctggttggaggggacattccctgctctggtc

SEQ ID NO: 17 - DNA, 20 bp, Artificial Sequence
  Primer oSL46
accttgatgattgcgctctt

SEQ ID NO: 18 - DNA, 20 bp, Artificial Sequence
  Primer oSL47
tgtcccagtgagagttgcag

SEQ ID NO: 19 - DNA, 20 bp, Artificial Sequence
  Primer oSL6
aaaagcaccgactcggtgcc

SEQ ID NO: 20 - DNA, 24 bp, Artificial Sequence
  Primer P0083
atggatctgatttagagatgaggc

SEQ ID NO: 21 - DNA, 23 bp, Artificial Sequence
  Primer P0084
ctatgcaggcaacaccattttct

SEQ ID NO: 22 - DNA, 20 bp, Artificial Sequence
  Primer P0074
catggacacgagtctgctct

SEQ ID NO: 23 - DNA, 20 bp, Artificial Sequence
  Primer P0075
gctgcctccacctttaagtc

SEQ ID NO: 24 - DNA, 20 bp, Artificial Sequence
  Primer P0081
ccctggagaagagctacgag

SEQ ID NO: 25 - DNA, 20 bp, Artificial Sequence
  Primer P0082
aaggtagtttcgtggatgcc

SEQ ID NO: 26 - DNA, 16 bp, Artificial Sequence
  Primer H2 fwd
gatgacrtccggcayc

SEQ ID NO: 27 - DNA, 18 bp, Artificial Sequence
  Primer H2 rev
cagttcctgcgccttgat

SEQ ID NO: 28 - DNA, 21 bp, Artificial Sequence
  H2 Probe
tgcaatcgatccagacggctt

SEQ ID NO: 29 - DNA, 20 bp, Artificial Sequence
  Primer SU1-Bel fwd
tctttgtttgcaatcgatcc

SEQ ID NO: 30 - DNA, 20 bp, Artificial Sequence
  Primer SU1-Bel rev
ggcgcactgtatgactgact

SEQ ID NO: 31 - DNA, 17 bp, Artificial Sequence
  SU1-Bel probe
ccggaactgcgctttca

SEQ ID NO: 32 - DNA, 20 bp, Artificial Sequence
  Primer DAI fwd
ggatactatcacgggcggta

SEQ ID NO: 33 - DNA, 20 bp, Artificial Sequence
  Primer DAI rev
ggcacgccatacaattctta

SEQ ID NO: 34 - DNA, 20 bp, Artificial Sequence
  Primer P0239
tacatgggtgacctgtctgg

SEQ ID NO: 35 - DNA, 20 bp, Artificial Sequence
  Primer P0240
acagctgcttgaacttggtg

SEQ ID NO: 36 - DNA, 64 bp, Artificial Sequence
  Edited CD163 gene sequence
attgtctccagggaaggacagggaggtctagaatcggctaagcccacgtagggttaggtagtca

(19) United States
(12) Patent Application Publication
(10) Pub. No.: US 2020/0045945 A1
(43) Pub. Date: Feb. 13, 2020

(54) Swine Comprising Modified CD163 and Associated Methods

(71) Applicant: The University Court of the University of Edinburgh, Edinburgh, Midlothian (GB)

(72) Inventors: Simon Geoffrey Lillico, Edinburgh Lothian (GB); Alan Archibald, Edinburgh Lothian (GB); Christopher Bruce Alexander Whitelaw, Biggar South Lanarshire (GB); Christine Tait-Burkard, Bo'ness (GB); Tahar Ait-Ali, Ivry-sur-Seine (FR)

(21) Appl. No.: 16/342,650

(22) PCT Filed: Oct. 17, 2017
(86) PCT No.: PCT/EP2017/076460

§ 371 Date: Apr. 17, 2019

(30) Foreign Application Priority Data
Oct. 17, 2016 (GB) 1617559.8

(51) Int. Cl. A01K 67/027; C07K 14/705; C12N 15/10; C12N 15/85; C12N 15/90; C12N 5/0786

(52) U.S. Cl. CPC A01K 67/0275; C07K 14/70596; C12N 15/102; C12N 15/8509; A01K 2267/02; C12N 5/0645; A01K 2217/05; A01K 2227/108; C12N 15/907

(57) ABSTRACT

The present invention relates to genetically edited swine which produce CD163 protein in which the scavenger receptor cysteine-rich 5 (SRCR5) domain (also known as CD163 domain 5) has been deleted. Such swine have been found to be healthy and do not exhibit negative properties, and are resistant to PRRSV infection. CD163 expressed in the edited swine also demonstrates retention of the ability to function as a haemoglobin-haptoglobin scavenger. Methods of producing such swine are also provided.

The present invention relates to genetically edited swine which produce CD163 protein in which the scavenger receptor cysteine-rich 5 (SRCR5) domain has been deleted. Such swine have been found to be healthy and do not exhibit negative properties, and are resistant to PRRSV infection. Moreover, the CD163 protein without the SRCR5 retains the ability to function as a haemoglobin-haptoglobin scavenger. The invention also relates to methods of producing such swine.

### BACKGROUND OF THE INVENTION

Porcine Reproductive and Respiratory Syndrome Virus (PRRSV) is a virus that causes a disease of pigs, called Porcine Reproductive and Respiratory Syndrome (PRRS).

This economically important disease, which is endemic in many pig producing countries, causes reproductive failure in breeding stock and respiratory tract illness in young pigs. Initially referred to as “mystery swine disease” and “mystery reproductive syndrome,” it was first reported in 1987 in North America and Central Europe. It is estimated that the disease costs the United States swine industry around $650 million annually.

PRRSV enters macrophages via a set of macrophage cell surface markers: CD169 and CD163. The role of CD169/sialoadhesin was discovered by the group of Hans Nauwynck in Ghent. The role of CD163 was discovered by scientists working with Pfizer (Calvert et al. 2007). Calvert et al. (2007) demonstrated that transfection of any non-susceptible cells with CD163 can render the cells susceptible to PRRSV. That has allowed for the generation of vaccine strains without the need of using Marc-145 cells.

Van Gorp et al. (“Susceptible cell lines for the production of porcine reproductive and respiratory syndrome virus by stable transfection of sialoadhesin and CD163”, BMC Biotechnology 2010, 10:48) have demonstrated that the domains 5 to 9 of the CD163 protein are important for the PRRSV entry into non-susceptible cells and highlighted that domain 5 may be critical.

Das et al. (“The Minor Envelope Glycoproteins GP2a and GP4 of Porcine Reproductive and Respiratory Syndrome Virus Interact with the Receptor CD163”, JOURNAL OF VIROLOGY, February 2010, p. 1731-1740) have demonstrated that that the PRRSV glycoprotein GP2A and GP4 interact physically with CD163.

US 20050271685 held by Pfizer (Zoetis) suggests that the use of CD163 molecule can make cells susceptible to PRRSV and ASFV.

WO 2012/158828 describes PRRS resistant animals in which the SIGLEC1 and/or CD163 genes have been inactivated. CD163, however, has roles in normal physiological activities. It is therefore undesirable to inactive this gene as it may have undesirable and unforeseeable knock-on effects on the animal.

There remains a need for improvements in the prevention and treatment of PRRSV.

The present inventors have succeeded in generating genetically edited swine which produces CD163 in which the scavenger receptor cysteine-rich 5 (SRCR5) domain (also known as CD163 domain 5) has been deleted. Swine produced by the inventors have been found to be healthy and do not exhibit negative properties. Experiments conducted by the inventors have shown that the swine demonstrate resistance to PRRSV infection. CD163 expressed in the edited swine also demonstrates retention of the ability to function as a haemoglobin-haptoglobin scavenger.

### STATEMENTS OF THE INVENTION

According to a first aspect of the present invention, there is provided a genetically edited swine, the swine comprising an edited genome wherein the edit results in the deletion of SRCR5 domain from CD163 protein produced by the swine. In other words, the genetically edited swine produces a modified form of the CD163 protein in which SRCR5 (also referred to in context as domain 5) is absent.

Preferably the swine is a pig (Sus scrofa), and most preferably a domestic pig (Sus scrofa domesticus or Sus domesticus).

Suitably the swine comprises an edited genome wherein the edit results in the deletion of SRCR5 from CD163 protein produced by the animal, and wherein all of the other CD163 domains are present and their amino acid sequences are unaltered. Accordingly, the swine suitably produces CD163 in which SRCR5 is absent, but SRCR domains 1 to 4 and 6 to 9 are unaltered, as are the transmembrane segment and cytoplasmic domain. The present inventors have found, surprisingly, that a CD163 protein in which SRCR5 has been deleted can retain its physiological function as a hemoglobin-haptoglobin scavenger, but generates high levels of resistance to infection by PRRSV in cells bearing the modified CD163 protein.

Accordingly, in certain embodiments of the present invention the CD163 protein expressed from the edited genome preferably remains substantially functional. ‘Substantially functional’ in this context refers to the protein retaining physiological functions that are not dependent on the SRCR5 domain. Suitably the modified CD163 protein is substantially functional, in that it is able to function as a haemoglobin-haptoglobin scavenger. The ability of a CD163 protein to function as a haemoglobin-haptoglobin scavenger can readily be assessed according to the methodology described herein, i.e. based upon the ability of peripheral blood monocyte-derived macrophages from edited swine to scavenge haemoglobin-haptoglobin. The ability of the CD163 protein to function as a haemoglobin-haptoglobin scavenger is indicative that the CD163 protein is correctly folded and functional despite the deletion of the SRCR5 domain.

SRCR5 of CD163 has the following amino acid sequence:

(SEQ IN NO: 2)
HRKPRLVGGDIPCSGRVEVQHGDTVVGTVCDSDFSLEAASVLCRELQCGT
VVSLLGGAHFGEGSGQIWAEEFQCEGHESHLSLCPVAPRPDGTCSHSRDV
GVVCS.

Accordingly, the modified CD163 protein produced by the edited swine suitably lacks the abovementioned amino acid sequence, i.e. SEQ ID NO:2. Suitably the CD163 protein produced by the edited swine has no further changes to the wild type amino acid sequence.

The swine is preferably homozygous or biallelic for the genome edit that results in the deletion of SRCR5 from CD163 produced by the animal. ‘Homozygous’ in this context means that the swine comprises the same edit within the CD163 gene on both chromosomes, i.e. it has identical alleles on both chromosomes. ‘Biallelic’ in this context means the swine has different edits on each chromosome, but wherein both of the edits result in a desirable edit to CD163, i.e. which results in the deletion of SRCR5 from CD163 protein produced by the animal.

Preferably all cells of the animal comprise the edited genome. In some cases, however, the animal can exhibit mosaicism, with some cells comprising the edited genome, and other cells not comprising the edited genome. PRRSV infects macrophages, and thus provided macrophages, and their progenitor cells, do not express CD163 which comprises SRCR5, the animals will be resistant to PRRSV infection.

It is generally preferred that the swine does not produce any CD163 which comprises SRCR5, i.e. all cells of the animal are homozygous or biallelic for the genome edit that results in the deletion of SRCR5 from CD163 produced by the swine.

It will be apparent to the skilled person that a genetically edited swine of the present invention can be a swine that has been directly subjected to a gene editing methodology as described herein, or a descendent of such a swine that retains the edited genome. Indeed, a swine that has been subjected to a gene editing methodology will in some cases be heterozygous, and will be bred to arrive at a homozygous or biallelic descendent.

Suitably the genome is edited such that the sequence which codes for SRCR5 is absent from the mRNA (preferably the mature mRNA) produced from the edited CD163 gene. This can be achieved by an edit that deletes exon 7, which encodes the SRCR5 domain of the CD163 protein, from the CD163 gene, or by an edit that results in the removal of the RNA sequence encoded by exon 7 from the transcript from the edited CD163 gene, e.g. as a result of splicing during the formation of mRNA.

Accordingly, in certain embodiments of the invention exon 7 of the CD163 gene is deleted. Deletion of exon 7 of the CD163 gene will of course result in the deletion of SRCR5 from the encoded CD163 protein.

In certain embodiments of the invention the splice acceptor site located at the 5′ of exon 7 is inactivated. Inactivation of the splice acceptor site at the 5′ end of exon 7 results in exon 7 being spliced out of the mRNA produced form the edited CD163 gene, thus deleting SRCR5 from the CD163 protein that is obtained from the mRNA when it is translated.

In embodiments of the invention where the swine comprises an edited genome in which exon 7 of the CD163 gene has been deleted, this can be achieved in various ways. For example, the deletion can be limited to exon 7, or the deletions can extend beyond exon 7 into flanking intronic regions (i.e. into introns 6 and 7). It is typically preferred that the entirety of exon 7 is deleted.

Suitably the edited genome is edited such that exon 7 has been deleted, but there are no other changes to other coding regions of the CD163 regions. In particular, it is typically preferred that no other exons of CD163 have been altered compared to the unedited genome. Accordingly, exons 1 to 6 and 8 to 16 are preferably unaltered.

In some embodiments, exon 7 and portions of introns 6 and 7, which flank exon 7, are deleted, but there are no other alterations in the remaining regions of the CD163 gene.

Exon 7 spans from position 23392 to position 23706 with reference to SEQ ID NO:1. Accordingly, this region is suitably deleted in the edited swine genome.

It should be noted that, while positions or regions in the CD163 gene are described herein with reference to SEQ ID NO: 1, there will be variations in sequence of the CD163 between different individual swine (e.g. where single nucleotide polymorphisms (SNPs) or other polymorphisms occur), and thus individual swine may comprise a CD163 sequence that is slightly different to SEQ ID NO:1. References to positions or regions made with reference to SEQ ID NO: 1 are not meant to be strictly limiting, but should be construed as representative of the corresponding position in the CD163 gene of swine having any such sequence variation. The person skilled in the art could readily identify corresponding positions or regions in a CD63 gene comprising sequence variations using convention sequence alignment techniques, e.g. BLAST.

Suitably the edited genome is edited such that the splice site donor sequence in intron 6 (i.e. located at the junction of exon 6 and intron 6) and the splice site acceptor site in intron 7 (i.e. located at the junction of intron 7 and exon 8) are unaltered and remain functional. This facilitates correct splicing of the transcript produced from the edited CD163 gene. Accordingly, in embodiments of the present invention the sequences in the regions extending from position 10451 to position 10465, and from position 23783 to position 23824, with reference to SEQ ID NO: 1, are unaltered.

Suitably the genome is edited such that at least a portion of the region of the CD163 gene extending from position 10466 to 23782 with reference to SEQ ID NO:1 is deleted, wherein the portion comprises exon 7. Position 10466 lies immediately 3′ of the predicted splice donor site of intron 6 (i.e. at the 3′ end of exon 6). Position 23782 lies immediately 5′ of the predicted splice acceptor site of intron 7 (i.e. at the 5′ end of exon 8). The region can of course be smaller, provided that it comprises exon 7.

Suitably the genome is edited such that regions from positions 1 to position 10465 and from position 23783 or 23754 to position 32908, with reference to SEQ ID NO:1, are unaltered.

In certain embodiments of the present invention exon 7 is deleted along with up to 5000 bases, suitably up to 2000 bases, suitably up to 1000 bases, suitably up to 500 bases, suitably up to 300 bases or suitably up to 100 bases extending 5′ of the 5′ end of exon 7.

In certain embodiments of the present invention exon 7 is deleted along with up to 75 bases extending 3′ of the 3′ end of exon 7. This region extends from the 3′ end of exon 7 up to the predicted splice acceptor site at the 5′ end of exon 8. Suitably exon 7 is deleted along with up to 50 bases extending 3′ of the 3′ end of exon 7.

In one embodiment, the edited genome comprises a deletion of the region extending from approximately position 23060 to approximately position 23760, for example from position 23064 or 23065 to position 23753 or 23754, suitably 20365 to position 23753, with reference to SEQ ID NO:1.

In another embodiment, the edited genome comprises a deletion of the region extending from approximately position 23260 to approximately position 23760, for example from position 23267 or 23268 to position 237543 or 23754, suitably position 23268 to position 23753, with reference to SEQ ID NO:1.

In another embodiment, the edited genome comprises a deletion of the region extending from approximately position 23370 to approximately position 23760, for example from position 23373 or 23374 to position 237543 or 23754, suitably position 23374 to position 23753, with reference to SEQ ID NO:1.

In some embodiments of the invention the edited genome can comprise an inserted sequence not normally found at the relevant position (i.e. a heterologous inserted sequence). For example, when a section of the CD163 gene comprising exon 7 has been deleted, an inserted sequence can be present in the location in where the deletion occurred. Such insertions are a relatively common artefact of deletion of a sequence through gene editing. Such an insertion is typically inconsequential in the present context, and the inserted sequence is typically spliced out of the transcript produced from the gene. Accordingly, the inserted sequence typically does not result in any particular effect. The inserted sequence is generally not a sequence from the CD163 gene or any homologue or other related sequence. It is typically preferred that such a heterologous inserted sequence is not present in the edited genome.

In one particularly preferred embodiment the edited genome comprises a deletion of the region extending from position 23268 to position 23753, and wherein there is no insertion of a sequence at the location of the deletion. In such an embodiment, the edited genome of the swine at the former locus of the deleted exon 7 has the following sequence ATTGTCTCCAGGGAAGGACAGGGAGGTCTAGAATCGGCTAAGCCCAC∥GTAGGGTTAGGT AGTCA—SEQ ID NO:36 (wherein II represents the adjoining of the two cut sites that may be used to excise the region).

In certain embodiments of the invention, the swine comprises an edited genome in which the splice acceptor site in intron 6, i.e. located at the 5′ end of exon 7, of the CD163 gene has been inactivated. As mentioned above, inactivation of splice acceptor site at the 5′ end of exon 7 results in exon 7 being spliced out of the mRNA produced form the edited CD163 gene, thus deleting SRCR5 from the CD163 protein translated from the mRNA.

The predicted splice acceptor site in intron 6 extends from position 23378 to position 23416, with reference to SEQ ID NO:1. Accordingly, this sequence is suitably edited to inactivate the splice acceptor site.

The splice acceptor site can be partially or entirely deleted, or its sequence altered in any other suitable way so that it is no longer functional. Accordingly, in one embodiment the splice acceptor site is deleted. In another embodiment a sequence is inserted into the splice acceptor site that results in its inactivation. In another embodiment the splice acceptor site is modified such that it is inactivated, e.g. though a homology directed repair (HDR) mediated introgression event.

In one embodiment the sequence of the splice acceptor site is altered such that it comprises a restriction enzyme site. For example, the altered sequence can be altered such that it comprises an NcoI restriction enzyme site. However, there are a very large number of other restriction enzyme sites that could be provided. A benefit of introduction of a restriction enzyme site at the altered splice acceptor site is that it allows for easy analysis for the occurrence of a successful editing event.

In one embodiment the splice acceptor site is edited to alter the sequence from AATGCTATTTTTCAGCCCACAGGAAACCCAGG (SEQ ID NO: 3) to AATGCTATTTTTCgGCCatggGGAAACCCAGG (SEQ ID NO:4). The sequence changes are shown in lower case.

In preferred embodiments of the present invention the genetically edited swine has improved tolerance or resistance to PRRSV infection. Suitably the animal is resistant to PRRS infection. Deletion of SRCR5 from CD163 has been shown to result in CD163 expressing cells, particularly pulmonary alveolar macrophages (PAMs) and peripheral blood monocyte-derived macrophages (PMMs), becoming highly resistant to infection with PRRSV.

According to a second aspect of the present invention there is provided a genetically edited swine cell or embryo, wherein the edit results in the deletion of SRCR5 domain from CD163 protein produced by the swine cell or embryo. “Cell or embryo” in this context encompasses a somatic cell, germ cell, stem cell, gamete, zygote, blastocyst, embryo, foetus and/or donor cell.

The various features discussed with regard to the first aspect of the invention apply mutatis mutandis to the second aspect of the invention. For example, the nature of the various edits discussed above in respect of the swine apply equally to the edited cell or embryo.

According to a third aspect of the present invention there is provided a method of producing a genetically edited swine, the method comprising the steps of:
a) providing a swine cell;
b) editing the genome of the cell to create a genome modification which results in the deletion of SRCR5 from the CD163 protein; and
c) generating an animal from said cell.

The genome modification that results in deletion of SRCR5 from the CD163 protein can be deletion of exon 7 from the CD163 gene or the inactivation of the splice acceptor site associated with exon 7 of the CD163 gene, i.e. the splice acceptor site located at the 5′ end of Exon 7.

In step a) the swine cell can be any suitable cell. Suitably the swine cell can be a somatic cell, a gamete, a germ cell, a gametocyte, a stem cell (e.g. a totipotent stem cell or pluripotent stem cell) or a zygote.

Preferably the method is performed on a zygote. The term ‘zygote’ can be used in a strict sense to refer to the single cell formed by the fusion of gametes. However, it can also be used more broadly in the present context to refer to the cell bundle resulting from the first few divisions of the true zygote—this is more properly known as the morula.

It is preferred that the present method is at least initiated, and preferably completed, in the zygote at the single cell stage. This should result in all cells of the swine containing the same edit. It is, however, possible that the zygote may divide while the editing process is occurring. Depending on when the cell division occurs relative to the stage of the editing process, it is possible that one of the following will occur:
all cells will contain the same edit because they are derived from the single cell which was edited before division occurred (the edit can be to one allele or both alleles in the cells, and in some cases each allele could have same edited sequence, and in other cases they could have a different edited sequence, i.e. a biallelic editing even has occurred);
all cells will contain the same edit because identical editing events occurred in the daughter cells after division occurred;
a mosaic of cells with and without editing events is created because the cell divided before the editing event occurs and only one daughter cell was edited; and
a mosaic of cells with different edits is created because the cell divided and differing editing events happened in the daughter cells.

Editing can also be conducted after the first cell division, and the results may be of interest. However, this is generally less preferred where the desired result is a non-mosaic animal.

Step b) suitably comprises:
introducing a site-specific nuclease to the cell, the site-specific nuclease targeting a suitable target sequence in the CD163 gene;
incubating said cell under suitable conditions for said site-specific nuclease to act upon the DNA at or near to said target sequence; and
thereby induce an editing event in the CD163 gene that results in deletion of SRCR5 from the CD163 protein.

The editing event that results in deletion of SRCR5 from the CD163 protein can be deletion of exon 7 from the CD163 gene or the inactivation of the splice acceptor site associated with exon 7, i.e. located at the 5′ end of Exon 7.

In certain embodiments step b) suitably comprises introducing site-specific nucleases to the cell which are targeted to target sites flanking exon 7 of the CD163 gene so as to induce double-stranded DNA cuts on either side of exon 7 and thereby cause its deletion. The target sites are suitable in introns 6 and 7. Where a target site is in intron 6, the cutting site is preferably 3′ of the splice donor site at the 3′ end of exon 6. Where a target site is in intron 7, the cutting site is preferably 5′ of the splice acceptor site at the 5′ of exon 8.

In certain embodiments step b) suitably comprises introducing an upstream site-specific nuclease to the cell, the upstream site-specific nuclease targeting a target site upstream of exon 7 of the CD163, and introducing a downstream site-specific nuclease to the cell, the downstream site-specific nuclease targeting a target site downstream of exon 7 of the CD163. ‘Upstream’ in this context refers to a site which is located upstream of the 5′ end of exon 7 of the CD163 gene. Preferably the upstream target site is located in the region between the 5′ end of exon 7 and the splice donor site located at the 3′ end of exon 6. In some embodiments the upstream target site is located within 2000 bases (suitably within 1000 bases, 500 bases, 300 bases, 200 bases or 100 bases) upstream of the 5′ end of exon 7. The cutting site of a site-specific nuclease is typically within or very close to its target site, and thus the site-specific nuclease induces a DNA cut within 2000, 1000, 500, 300, 200 or 100 bases upstream of the 5′ end of exon 7. The cutting site of the site-specific nuclease is suitably in the region between the 5′ end of exon 7 and the splice donor site located at the 3′ end of exon 6.

The skilled person can readily target known site-specific nucleases (such as CRISPR/Cas9 or other CRIPR nucleases, TALENs or ZFNs) to any desired target sited in the regions discussed above. In the case of CRISPR/Cas9 or other CRIPR nucleases the method suitably comprises providing a guide RNA to direct the Cas9 protein to the desired target site. In the case of TALEN or ZFN it is the protein code of the site-specific nuclease that determines the binding site of the site-specific nuclease.

Exemplary upstream target sites which can be used in the case where the site-specific nuclease is CRISPR/Cas9, along with the associated cut location and sgRNAs are given below (cut locations are shown by the “|” symbol):

sgRNA (sgSL25)
(SEQ ID NO: 5)
TGAAAAATAGCATTTCGGTG,
CD163 gene target site and cut location:
(SEQ ID NO: 6)
CAC|CGAAATGCTATTTTTCA
sgRNA (sgSL26)
(SEQ ID NO: 7)
GAATCGGCTAAGCCCACTGT,
CD163 gene target site and cut location:
(SEQ ID NO: 8)
GAATCGGCTAAGCCCAC|TGT
sgRNA (sgSL27)
(SEQ ID NO: 9)
GTCCTCCATTTACTGTAATC,
CD163 gene target site and cut location:
(SEQ ID NO: 10)
GAT|TACAGTAAATGGAGGAC.

‘Downstream’ in this context refers to a site which is located at or near the 3′ end of exon 7 of the CD163 gene. Typically, a downstream site is located in intron 7. Preferably the downstream target site is located in the region between the 3′ end of exon 7 and the splice acceptor site located at the 5′ end of exon 8. In some embodiments the downstream target site is located within 75 bases or 50 bases 3′ of the 3′ end of exon 7. The cutting site of the site-specific nuclease is thus suitably within this defined region, so that the cut occurs 3′ of the 3′ end of exon 7, and 3′ of the 5′ end of the splice acceptor site located at the 5′ end of exon 8, for example, the cutting site of the site-specific nuclease is typically 5′ of the splice acceptor site located at the 5′ end of exon 8.

An exemplary downstream target site that can be used in the case where the site-specific nuclease is CRISPR/Cas9, along with the associated cut location and sgRNA sequence are given below (cut location is shown by the “|” symbol):

sgRNA (sgSL28)
(SEQ ID NO: 11)
CCCATGCCATGAAGAGGGTA,
CD163 gene targetsite and cut location:
(SEQ ID NO: 11)
CCCATGCCATGAAGAGGIGTA.

In certain embodiments step b) suitably comprises introducing a site-specific nuclease that targets the splice acceptor site associated with exon 7, i.e. located at the 5′ end of Exon 7.

Suitably a site-specific nuclease induces a double stranded cut within or near to the splice acceptor site associated with exon 7.

In some embodiments the site-specific nuclease induces a cut in the region extending from position 23378 to position 23416 with reference to SEQ ID NO:1, or at a position within 200, 100, 50 or 25 bases of said region in a 5′ or 3′ direction. In other words, the site-specific nuclease induces a double stranded cut in the predicted splice acceptor site associated with exon 7, or in flanking regions.

The skilled person can readily target known site-specific nucleases (such as CRISPR/Cas9, TALENs or ZFNs) to any desired target site in the regions discussed above. In the case of CRISPR/Cas9 and other CRIPR nucleases, the method suitably comprises providing a guide RNA to direct the Cas9 or other CRIPR nuclease protein to the desired target site. In the case of TALEN or ZFN it is the protein code of the site-specific nuclease that determines the binding site of the site-specific nuclease.

In the case of CRISPR/Cas9 mediated gene editing, suitable guide RNA sequences to target the splice acceptor site associated with exon 7 are as follows:

sgRNA 1:
(SEQ ID NO: 12)
AACCAGCCTGGGTTTCCTGT
sgRNA 2:
(SEQ ID NO: 13)
CAACCAGCCTGGGTTTCCTG

These two guide sequences result in the induction of double stranded cut sites at the following sequences at the 5′ end of exon 7 by Cas9 (cut locations are shown by the “|” symbol):

(SEQ ID NO: 14)
ACA|GGAAACCCAGGCTGGTT - using sgRNA 1
(SEQ ID NO: 15)
CAG|GAAACCCAGGCTGGTTG - using sgRNA 2

The site-specific nuclease suitably creates a single double stranded cut at the desired cutting site. In that case the splice acceptor site associated with exon 7 can be inactivated by non-homologous end joining (NHEJ) or by homology directed repair (HDR). Where HDR is the intended method of inactivation, an HDR template is provided. As is well-known in the art, the HDR template comprises a central portion, which contains the sequence intended to replace the normally occurring sequence, and flanking portions which are homologous to the normal sequence. The HDR template thus suitably comprises a central portion that has a sequence that, when introduced to the CD163 gene by HDR, inactivates the splice acceptor site.

An exemplary, but non-limiting, HDR template has the following sequence: GAAGGAAAATATTGGAATCATATTCTCCCTCACCGAAATGCTATTTTTCgGCCatggGGAAAC CCAGGCTGGTTGGAGGGGACATTCCCTGCTCTGGTC (SEQ ID NO:16) (lower case letters show the changes made compared to the unaltered sequence).

While the exemplary target sites set out above relate to the CRISPR/Cas9 site specific nuclease, it will be immediately apparent to the skilled person that many other target sites could be used, and also that other site specific nucleases (often referred to as ‘editors’ or ‘gene editors’ in this context) could be used. Suitable target sites for alternative site specific nucleases could readily be determined by the skilled person.

In preferred embodiments the site-specific nuclease comprises at least one zinc finger nuclease (ZFN), Transcription Activator-Like Effector Nuclease (TALEN), RNA-guided CRISPR nuclease (e.g. CRISPR/Cas9 or other CRISPR nuclease, such as CRISPR/Cpf), or a meganuclease.

The site-specific nuclease is typically capable of creating a double stranded break in the genomic DNA. This can be achieved with a number of site-specific nucleases, including, but not limited to, CRISPR/Cas or other CRISPR nuclease, ZFNs and TALENs.

In some embodiments the site-specific nuclease comprises a pair of cooperating site-specific nucleases, each of which are able to generate a single stranded break. Suitably the site-specific nuclease comprises a pair of cooperating ZFNs, TALENs or CRISPR ‘nickases’ (e.g. having a modified Cas9 or other nuclease capable of cutting only one DNA strand), which cooperate to generate a double stranded break in the genomic DNA. In such embodiments the target site suitably comprises a pair of half sites, with one of the pair binding at each half site. Thus, in some embodiments the site-specific nuclease comprises a pair of ZFNs, TALENs or RNA-guided CRISPR ‘nickases’ (e.g. having a modified Cas9 or other nuclease capable of cutting only one DNA strand), capable of causing a double stranded DNA break only when both members of the pair are present and form a heterodimer which is able to cut both strands of the DNA molecule. In some preferred embodiments the site-specific nuclease comprises a pair of ZFNs. The use of pairs of corresponding site-specific nucleases can have benefits in reducing off-target cuts

It should be noted that the site-specific nuclease can be introduced to a cell in any suitable form. For example, the nuclease can be provided directly into the cell as a functional protein. Alternatively, the nuclease can be provided into the cell in the form of a precursor or template from which the active nuclease is produced by the cell. In a preferred embodiment an mRNA encoding the nuclease is introduced into the cell, e.g. by injection. The mRNA is then expressed by the cell to form the functioning protein. Using mRNA in this way allows rapid but transient expression of the nuclease within the cell, which is ideal for the purposes of genetic editing. Where an RNA is used to target the site-specific nuclease, this can be provided in any suitable form.

It should also be noted that the term ‘nuclease’ is intended to cover any biological enzyme which creates a single or double stranded cut of a target nucleic acid. Accordingly, the term includes nickases and recombinases, as well as more conventional nucleases which cause single or double stranded breaks.

ZFN technology is described extensively in the literature and, inter alia, in the following patent documents: U.S. Pat. Nos. 6,479,626, 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, 6,479,626, 8,106,255, 20030232410, and 20090203140, all of which are incorporated by reference. ZFNs can be obtained commercially from Sigma-Aldrich (St. Louis, Mo., US) under the CompoZr® Zinc Finger Nuclease Technology branded products and services.

TALEN technology is described extensively in the literature and, inter alia, in the following patent documents: U.S. Pat. Nos. 8,420,782, 8,470,973, 8,440,431, 8,440,432, 8,450,471, 8,586,363, 8,697,853, EP2510096, U.S. Pat. Nos. 8,586,526, 8,623,618, EP2464750, US2011041195, US2011247089, US2013198878, WO2012/116274, WO2014110552, WO2014070887, WO2014022120, WO2013192316, and WO2010008562, all of which are incorporated by reference. TALENs can be obtained commercially from Thermo Fisher Scientific, Inc. (Waltham, Mass., US) under the GeneArt® TALs branded products and services (formerly marketed under the Life Technologies brand).

CRISPR/Cas technology is described extensively in the literature (e.g. Cong et al. ‘Multiplex Genome Engineering Using CRISPR/Cas Systems’, Science, 15 Feb. 2013: Vol. 339 no. 6121 pp. 819-823) and, inter alia, in the following patent documents: U.S. Pat. No. 8,697,359, US2010076057, WO2013/176772, U.S. Pat. No. 8,771,945, US2010076057, US2014186843, US2014179770, US2014179006, WO2014093712, WO2014093701, WO2014093635, WO2014093694, WO2014093655, WO2014093709, WO2013/188638, WO2013/142578, WO2013/141680, WO2013/188522, U.S. Pat. No. 8,546,553, WO2014/089290, and WO2014/093479, all of which are incorporated by reference. CRISPR/Cas systems can be obtained commercially from Sigma-Aldrich (St. Louis, Mo., US) under the CRISPR/Cas Nuclease RNA-guided Genome Editing suite of products and services, or from Thermo Fisher Scientific, Inc. (Waltham, Mass., US) under the GeneArt® CRISPR branded products and services. CRISPR/Cpf has also been widely described in the literature.

Of course, in this rapidly developing field other techniques for genetic editing are likely to become available. Such techniques could, in many cases, be readily adapted for use in the present invention.

With regard to step c) of, there are a range of well-known techniques in the art that can be used to produce animals from cells comprising genetic alterations. Such techniques include, without limitation, pronuclear microinjection (U.S. Pat. No. 4,873,191) or electroporation of embryos (Lo (1983) Mol. Cell. Biol. 3, 1803-1814), sperm-mediated gene transfer (Lavitrano et al. 25 (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Standard breeding techniques can be used to create animals that are homozygous or biallelic for a desired genetic edit from initially heterozygous founder animals. The specific description gives details of an exemplary, but not limiting, method for generating animals from an edited zygote. The present invention is not limited to any specific way of generating an animal from the edited cell produced in step b).

Step c) of the method can optionally involve cloning, e.g. somatic cell nuclear transfer (SCNT). In such an embodiment the genetic editing event is carried out on a somatic cell, after which the edited nucleus is transferred to an enucleated egg cell. Typically a population of somatic cells will be edited and cells in which a desired editing event has occurred will be used to provide donor nuclei for SCNT. Processes for SCNT have been well described in the art and would be known to the skilled person. However, it is an advantage of the present invention that editing can be performed without the need for cloning.

The method may suitably comprise crossing a swine produced from the genetically edited cell with another swine to obtain a descendent swine. Preferably the descendent swine is homozygous or biallelic for the genome edit that results in the deletion of SRCR5 from CD163 produced by the animal. This can be achieved, for example, by crossing two heterozygous swine, as is well known in the art. Thus, in some embodiments the method suitably comprises step d), crossing a swine produced in step c) (which can be heterozygous for the genome edit that results in the deletion of SRCR5 from CD163 produced by the animal), with another swine that is heterozygous for the genome edit that results in the deletion of SRCR5 from CD163 produced by the animal.

In certain embodiments, the method of the present invention comprises the steps of:
providing a swine zygote;
introducing a site-specific nuclease to the zygote, the site-specific nuclease targeting a suitable target sequence in the CD163 gene;
incubating said zygote under suitable conditions for said site-specific nuclease to act upon the DNA at or near to said target sequence and thereby induce an editing event in the CD163 gene that results in deletion of SRCR5 from the CD163 protein; and
generating an animal from said genetically edited zygote.

The genetically edited zygote can be grown to become an embryo and eventually an adult animal. As discussed above, if the editing event occurs in the single-cell zygote then all cells of this animal will therefore comprise the modified CD163 gene as all cells of the animal are derived from a single genetically edited cell. If the editing event occurs after one or more cell divisions then the resultant animal will likely be a mosaic for the editing event, in that it will have some cells derived from the edited cell and some cells derived from unedited cells.

The method may involve characterising the genetic editing event that has occurred. Suitable methods to achieve this are set out below.

The method can be performed on a plurality of zygotes and the method may involve selecting zygotes in which the desired genetic modification has been achieved.

Preferably the swine produced according to the methods of the present invention is homozygous or biallelic for the genome edit that results in the deletion of SRCR5 from CD163 produced by the animal. This can be achieved directly as a result of the editing process of step b), or by a subsequent crossing step between two heterozygous swine.

According to the fourth aspect of the present invention there is provided a method of producing a genetically edited swine cell or embryo, the method comprising the steps of:
providing a swine cell or embryo;
editing the genome of the cell or cells within the embryo to create a genomic edit which results in the deletion of SRCR5 of CD163.

The various features discussed with regard to the third aspect of the invention apply to the fourth aspect of the invention mutatis mutandis.

According to a fifth aspect of the invention there is provided an animal, cell or embryo produced according to the third or fourth aspects of the invention.

According to a sixth aspect of the present invention, there is provided a method of modifying a swine to increase its resistance or tolerance to PRRSV comprising editing the genome of cells in the swine to create a modification which results in the deletion of SRCR5 domain of the CD163 protein.

According to a seventh aspect of the present invention, there is provided a swine or a cell of a swine which expresses or bears a CD163 protein in which the SRCR5 domain has been deleted. The cell may suitably be a macrophage, and in some cases can be a peripheral blood monocyte-derived macrophages (PMM) or pulmonary alveolar macrophage (PAM).

Embodiments of the present invention will now be described, by way of non-limiting example, with reference to the accompanying drawings.

### BRIEF DESCRIPTION OF THE FIGURES

FIG. 1: Generation of an Exon 7 deletion in CD163 using CRISPR/Cas9. A) Schematic of the CD163 gene in the pig genome on chromosome 5. Indicated in red are the 16 exons encoding the CD163 mRNA, in varied colors underneath are the 9 scavenger receptor cysteine-rich (SRCR) domains that form the “pearl on a string” structure of the CD163 protein. Excision of exon 7 using two guide RNAs (sgSL26 & sgSL28) located in the flanking introns should result in SRCR 5 removal from the encoded protein. Indicated are also the locations of sgRNAs SL25 and SL27. B) In vitro assessment of guide RNAs sgSL25, sgSL26, sgSL27, and sgSL28. PK15 cells were transfected with either a single plasmid encoding a guide RNA+Cas9 or co-transfected with combination of two such plasmids. Transfected cells were identified by GFP expression and isolated by FACS. Cutting efficiency of single guide RNA transfection was assessed by a Cell surveyor assay. Relative efficiency of exon7 deletion upon double transfection was assessed by PCR. C) Schematic of the Cas9/guide RNA injection into zygotes. The injection mix was injected into the cytoplasm of zygotes and contained uncapped, non-polyadenylated guide RNAs sgSL26 and sgSL28, as well as capped, polyadenylated Cas9 mRNA.

FIG. 2: Excision of Exon7 results in an SRCR5 CD163 deletion in pigs. A) Representative photos of the male sibling pigs with three different ΔSRCR5 genotypes at 5 months of age. Left, wild type pig 628, middle, heterozygous pig 627, and right, biallelic pig 629. B) Genotyping of pulmonary alveolar macrophages (PAMs). DNA was extracted from PAMs and genotype assessed by PCR across Intron 6 to Exon 8. The unmodified genome PCR is predicted to result in a 900 bp product, whilst exon 7 deletion should result in a 450 bp PCR product. C) RNA phenotype of pulmonary alveolar macrophages. RNA was extracted from PAMs, converted into cDNA using oligo(dT) primer, and analyzed by PCR across Exons 4-9. The unmodified cDNA should result in a 1686 bp product, whilst the exon7 deletion is expected to yield a 1371 bp product. D) Protein phenotype of CD163 from PAMs. PAM cells were lysed with reducing SDS sample buffer and CD163 expression analyzed by western blot. E) CD163 mRNA levels in PAMs. RNA was extracted from the same number of PAM cells, DNA removed by DNase treatment, and RNA quantified by 1-step RT-qPCR. Expression levels were normalized using β-Actin expression levels and to the highest CD163 expressing animal. Error bars represent SEM, n=3*2.

FIG. 3: ΔSRCR5 pulmonary alveolar macrophages (PAMs) are fully differentiated and express macrophage-specific markers. PAMs isolated by bronchoalveolar lavage were assessed by staining with various macrophage markers and FACS analysis. Staining against the native structure of surface expressed CD163 (right hand peak) relative to an isotype control staining (left hand peak).

FIG. 4: ΔSRCR5 pulmonary alveolar macrophages (PAMs) are not susceptible to infection with PRRSV genotype 1. A-C) PAMs from wild-type (wt, left hand two columns), heterozygous (het, middle two columns), and ΔSRCR5 (biallelic or homologous SRCR5 deletion) (right hand two columns) animals were inoculated at MOI (multiplicity of infection)=1 of PRRSV genotype 1, subtype 1 (strain H2, A), subtype 2 (strain DAI, B), and subtype 3 (strain SU1-Bel, C). 19 hours post infection (hpi) cells were detached, fixed and stained with an anti PRRSV-N protein antibody and CD163. Infection was quantified by FACS analysis. Over 98% of infected macrophages were qualified as CD163 positive. Infection levels were statistically analyzed using an unpaired t-test of all wt against all het or all bia/hom. Error bars represent SEM, n=3. D-F) Replication growth curves of PRRSV genotype 1, subtype 1 (strain H2, C), subtype 2 (strain DAI, D), and subtype 3 (strain SU1-Bel, F). PAMs from wild-type (628 filled circle, 633 open circle), heterozygous (627 filled square, 633 open square), and ΔSRCR5 (629 triangle pointing down, 630 triangle pointing up) animals were inoculated at MOI=0.1 of the respective strain. Cell supernatant was collected at indicated time points to measure the released viral RNA by RT-qPCR. Error bars represent SEM, n=3*2. G-J) Quantification of infectious particles produced at 48 hpi by TCID50 analysis. Cell supernatant collected at the 48 hpi time point of infection of the time-course experiment was analyzed for infectious viral particle production quantified by 50% tissue culture infective dose (TCID50). Infection levels were statistically analyzed using an unpaired t-test of all wt against all het or all ΔSRCR5. Error bars represent SEM, n=3. Columns are the same as for panes A-C.

FIG. 5: ΔSRCR5 peripheral blood monocyte-derived macrophages (PMMs) are fully differentiated and express macrophage-specific markers. Peripheral blood monocytes were isolated from the blood of the wild-type, heterozygous, and ΔSRCR5 animals. Following cultivation in the presence of Recombinant human Colony Stimulating Factor 1 (rhCSF1) for seven days PMMs were analyzed by FACS. A) Co-staining with CD14-FITC and CD16-PE antibodies recognizing the native structure of the proteins (contour plots; 628 and 633=wild type, 627 and 364=heterozygous, 629 and 630=ΔSRCR5) relative to isotype controls (isotype controls are represented the lower left contour plot in each graph, and the macrophage-specific markers are the upper right contour plot). B) Co-staining with CD169-FITC and CD172a-PE antibodies recognizing the native structure of the proteins (upper right contour plots) relative to isotype controls (lower left). C) Co-staining with SWC9 (CD203a)-FITC and CD151-RPE antibodies recognizing the native structure of the proteins (upper right contour plots) relative to isotype controls (lower left). D) Staining against the native structure of surface expressed CD163 (right hand plot) relative to an isotype control staining (left hand plot).

FIG. 6: ΔSRCR peripheral blood monocyte-derived macrophages (PMMs) still function as hemoglobin-haptoglobin (Hb-Hp) scavengers. A) Induction of Heme oxygenase 1 (HO-1) expression by Hb-Hp uptake. PMMs were incubated for 24 hours (h) in presence of 100 μg/ml Hb-Hp. RNA was isolated from cells and levels of heme oxygenase 1 (HO-1) mRNA determined by RT-qPCR (outlined bars uninduced, filled bars Hb-Hp uptake induced; left hand two columns=wild type, middle two columns=heterozygous, right hand two columns=ΔSRCR5). Expression levels were normalized using β-Actin expression levels and to the level of unstimulated HO-1 mRNA expression of each animal. Uninduced versus induced levels of HO-1 expression were analyzed by an unpaired t-test. Error bars represent SEM, n=3. B) PMMs were incubated for 24 h in presence of 100 μg/mol Hb-Hp. PMMs were lysed with reducing SDS sample buffer and HO-1 protein expression analyzed by western blot. C) Quantification of Hb-Hp uptake. PMMs were incubated in presence of 10 μg/ml HbAF488-Hp for 30 minutes (min). Uptake of HbAF488-Hp was measured by FACS analysis (right hand peaks) relative to isotype controls (left hand peaks). Hb-Hp uptake was also visualised. PMMs were incubated for 30 min with 10 μg/ml HbAF488-Hp. Cells were fixed, permeabilized and stained against CD163 and with DAPI (data not shown).

FIG. 7: ΔSRCR5 peripheral blood monocyte-derived macrophages (PMMs) are not susceptible to infection with PRRSV genotype 1. A-C) PMMs from wild-type (left hand two columns), heterozygous (middle two columns), and ΔSRCR5 (right hand two columns) animals were inoculated at MOI=1 of PRRSV genotype 1, subtype 1 (strain H2, A), subtype 2 (strain DAI, B), and subtype 3 (strain SU1-Bel, C). 19 hpi cells were detached, fixed and stained with anti PRRSV-N protein and CD163 antibodies. Infection was quantified by FACS analysis. Infection levels were statistically analyzed using an unpaired t-test of all wt against all het or all ΔSRCR5. Error bars represent SEM, n=3. D-F) Replication of PRRSV on PMMs in long-term infections with genotype 1, subtype 1 (strain H2, D), subtype 2 (strain DAI, E), and subtype 3 (strain SU1-Bel, F). PMMs from wild-type (628 filled circle, 633 open circle), heterozygous (627 filled square, 633 open square), and ΔSRCR5 (629 triangle pointing down, 630 triangle pointing up) animals were inoculated at MOI=0.1 of the respective strain. Cell supernatant was collected at indicated time points to measure the released viral RNA by RT-qPCR. Error bars represent SEM, n=3*2.

FIG. 8: PRRSV infection of ΔSRCR5 pulmonary alveolar macrophages (PAMs) is halted prior to the formation of the replication/transcription complex. PAMs from wild-type (top panels), heterozygous (middle panels), and ΔSRCR5 (bottom panels) animals were inoculated at MOI=2 with PRRSV genotype 1, subtype 1 (strain H2, top row), subtype 2 (strain DAI, middle row), and subtype 3 (strain SU1-Bel, bottom row). 22 hpi cells were fixed and stained with an anti PRRSV-nsp2 antibody, DAPI, and phalloidin.

FIG. 9: Genotypes of founder animals. A) Genotype of founder animal 310 (f). The genotype of 310 was assessed by PCR across intron 6 to exon 8. DNA template was extracted from two ear biopsies, a tail clipping and from a buffy coat. The unmodified genome PCR is predicted to result in a 900 bp product, whilst the exon 7 deletion should result in a 450 bp PCR product. Displayed as well is the PCR result from one of her unmodified siblings (311) as a control. B) Specific genotype of 310 as assessed by Sanger sequencing of the PCR product across intron 6 to exon 8. C) Genotype of founder animals 345 (m), 346 (f), and 347 (f). The genotype of the animals was assessed by PCR across intron 6 to exon 8. DNA template was extracted from two ear biopsies, one of them only containing ear tip (epidermis and dermis), buffy coat and pulmonary alveolar macrophages. Genotypes from the different tissue samples reveal a mosaicism of heterozygous and homozygous tissues. Displayed as well are the PCR result from unmodified sibling control animals 342, 343 and 344. B) Specific genotype of 345, 346, and 347 as assessed by Sanger sequencing of the PCR product.

FIG. 10: Genotypes of litter from 310×345 mating. A) The genotype of piglets 627-635 and ovl/SB (Ovl=overlaid pig, SB=stillborn) piglets was assessed by PCR across intron 6 to exon 8. DNA template was extracted from ear biopsy. The unmodified genome PCR is predicted to result in a 900 bp product, whilst the exon 7 deletion should result in a 450 bp PCR product. B) Family tree with indicated genotype. On the image the heterozygous genotype of 310 and 345 is represented by shading, dark grey indicates the edited allele and light grey indicates unmodified (alleles). 310 and 345 are represented as heterozygous despite mosaicisms found in both animals as this represents the genotype found in the germline. 630 is homozygous for the edited allele from 310. 627, 634, 635, OVL/SB1, OVL/SB2, OVL/SB4 are heterologous with one edited allele from 345 and the other unaltered. 629 is heterozygous with one edited allele from 345 and one from 310.

FIG. 11: Generation of ΔSRCR5 pigs and experimental set-up. A) Genome editing to generate ΔSRCR5 pigs. Genome-edited founder animals were generated by zygote injection of CRISPR/Cas9 editing reagents using two guide RNAs, sgSL26 and sgSL28, in combination to generate a deletion of exon 7 in CD163. Animals were breed to generate an F1 and an F2 generation focusing on one genotype showing clean re-ligation at the cutting sites of both guide RNAs. Homozygous F2 generation animals carry this genotype in both alleles (bottom). B) Structure prediction and expression of ΔSRCR5 in pulmonary alveolar macrophages of F2 animals. Left: Protein structure prediction using RaptorX eludes towards an intact protein product upon deletion of SRCR5. C) Experimental design of challenge study. 4 homozygous (green) and 4 wildtype (orange) siblings from heterozygous/heterozygous mating of the F1 generation animals were co-housed from weaning. Genotypes were confirmed by PCR amplification across exon 7 (see FIG. 1A) and by Sanger sequencing. Piglets were co-housed after weaning and, after acclimation to the specific pathogen-free unit for 1 week, inoculated intranasally with 5E6 TCID₅₀ of the PRRSV-1, subtype 2 strain BOR-57 at day 0 & day 1 of the challenge at age 7-8 weeks for 14 days.

FIG. 12: ΔSRCR5 pigs show normal serum levels of soluble C163. Serum samples collected 2 weeks and on day 0 prior to the challenge were assessed towards the level of sCD163 present using a commercial ELISA. n=2*2*3, displaying min/max and 90 percentile. Statistical analysis using an unpaired t-test showed no significant difference.

FIG. 13: ΔSRCR5 pigs show no clinical signs, virus replication or pathology of a PRRSV-1 infection. A) Rectal temperature of ΔSRCR5 (solid circles) and wildtype piglets (filled squares) during the challenge with BOR-57. Rectal temperatures were measured daily during feeding. Error bars represent SEM, n=4. B) Average daily weight gain based on weight measurements at day 0, 7, and 14 of the challenge. A&B; Statistical analysis was performed using a two-way ANOVA & Sidak's multiple comparison test. C) Viremia during the challenge with BOR-57. Serum samples were collected at day 0, 3, 7, 10, and 14 from the jugular vein using vacuum tubes, viral RNA isolated and quantified using RT-qPCR with primers specific to ORF5 of BOR-57. D) Antibody response to PRRSV-1 during the challenge. Serum samples were analyzed towards the presence of PRRSV antibodies using the IDEXX PRRSV X3 ELISA test. <0.40=negative; 0.4=positive. E) Lung and Lymph node pathology, histopathology and immunohistochemistry scores. Left bars represent the ΔSRCR5, right bars the wildtype pigs. Lung pathology was assessed in a blind fashion and a subjective score for severity of gross lung lesions using an established scoring system was applied (scale 0-100). Lung histopathology sections were scored for the presence and severity of interstitial pneumonia ranging from 0 to 6 (0, normal; 1, mild multifocal; 2, mild diffuse; 3, moderate multifocal; 4, moderate diffuse; 5, severe multifocal; 6, severe diffuse). Immunohistochemistry staining against PRRSV-N of lung and lymph node sections was scored ranging from 0-3 (0, no signal; 1, low numbers of positive cells; 2, moderate numbers of positive cells; 3, abundant). F) Lung histology and immunohistochemistry. Top: formalin-fixed, paraffin-embedded, haemotoxylin and eosin stained lung sections from the necropsy on day 14 post challenge. Left: ΔSRCR5, right: wildtype piglets. The scale bar represents 100 μm. Bottom: formalin-fixed, paraffin-embedded immunohistochemical stain against PRRSV antigen and hematoxylin counterstain. Left: ΔSRCR5, right: wildtype piglets. The scale bar represents 50 μm. G) Lung pathology. Lungs from pigs at necropsy 14 days post challenge; left, lungs from two ΔSRCR5 pigs and right, lungs from two wildtype pigs.

FIG. 14: ΔSRCR5 pigs show normal cytokine levels and no cytokine response to BOR-57 PRRSV infection. Cytokine levels in serum samples collected prior to challenge on day 0, and challenge days 3, 7, 10, and 14 were measured using cytokine antibody arrays. ΔSRCR5=solid circles and wildtype piglets=filled squares. A) Interferon α (IFNα), B) Interleukin 17A (IL-17A), C) Interleukin 1 receptor antagonist (IL-1ra), D) Interleukin 4 (IL-4), E) Interleukin 6 (IL-6), F) Interleukin 4 (IL-4), G) Monokine induced by gamma interferon (MIG/CXCL9), H) Macrophage inflammatory protein-1β (MIP-1β/CCL4), I) Chemokine ligand 3-like 1 (CCL3L1), J) Granulocyte macrophage colony-stimulating factor (GM-CSF), K) Tumor necrosis factor alpha (TNFα), L) Interleukin 12 (IL-12), M) Interleukin 1 beta (IL-1β), N) Interleukin 10 (IL-10), 0) Transforming growth factor beta 1 (TGFβ1), P) Interferon gamma (IFNγ), Q) Interleukin 18 (IL-18), R) Platelet endothelial cell adhesion molecule (PECAM-1/CD31), S) Interleukin 1 alpha (IL-1α), T) Interleukin 13 (IL-13). Error bars represent SEM, n=2*4. Statistical analysis was performed using a two-way ANOVA & Sidak's multiple comparison test.

### SPECIFIC DESCRIPTION OF EMBODIMENTS OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “swine”, or variants thereof, as used herein refers to any of the animals in the Suidae family of even-toed ungulates including animals in the genus Sus and other related species, including the peccary, the babirusa, and the warthog.

The term “pig” or variants thereof as used herein refers to any of the animals in the genus Sus. It includes the domestic pig (Sus scrofa domesticus or Sus domesticus) and its ancestor, the common Eurasian wild boar (Sus scrofa). For the present purposes, the domestic pig is considered to be a sub-species of the species Sus scrofa. It does not include the peccary, the babirusa, and the warthog.

The term “domestic pig”, or variants thereof, as used herein refers to an animal of the sub-species Sus scrofa domesticus.

The term “site-specific nuclease”, or variants thereof, as used herein refers to engineered nucleases which can be configured to cut DNA at a desired location. Such site-specific nucleases are also known as engineered nucleases, targetable nucleases, genome editing nucleases, molecular scissors, and suchlike. Examples of site-specific nucleases include zinc finger nucleases (ZFNs), Transcription Activator-Like Effector Nucleases (TALENs), the CRISPR/Cas system (CRISPR/Cas), and meganucleases, such as hybrid meganucleases.

“Genetically edited” or “genetically modified” when used in relation to subject biological material, refers to the fact that the subject biological material has been treated to produce a genetic modification thereof compared to control, e.g. wild type, biological material.

“Target site” refers to the site having a nucleic acid sequence to which a site-specific nuclease binds. When the site-specific nuclease bind at a target site it acts to cut the DNA within or adjacent to the target site (this can be achieved by a single site-specific nuclease, or a corresponding pair or nucleases, in which case there will be two so-called “half-sites”, as desired), the location of the cut being referred to as the “cut site” or “cutting site”. Where a target site is defined for a site-specific nuclease above, the cut site is suitably with the target site, or adjacent to the target site. Where the target site is mentioned as being near or adjacent to a specific feature in the genome, e.g. a feature to be deleted or preserved in an editing event (such as exon 7 or a splice site), the cutting site should be located so as to achieve the desired outcome, i.e. it results in deletion or preservation of the feature, as desired. Site-specific nucleases can be designed to target any desired target site; for example, with CRISPR/Cas9 this can be achieved using a suitable sgRNA, and for ZFN or TALENs suitable proteins can be designed and obtained from commercial sources.

“ΔSRCR5” refers to an animal, typically a swine, which comprises a biallelic or homozygous CD163 SRCR5 deletion.

“Unaltered” with reference to a nucleic acid sequence (such as a region of the genome or a gene) means that the sequence has not been altered from the wild type sequence.

“Tolerance or resistance”—an animal can be said to be more tolerant or resistant to PRRSV infection when the mortality rate, morbidity rate, the proportion of animals showing significant morbidity (e.g. weight loss or decreased growth rate), the level of morbidity or the duration of morbidity is reduced when animals are challenged with PRSSV infection. Any statistically significant reduction (e.g. 95% confidence, or 99% confidence using an appropriate test) in the mortality or morbidity between a population of genetically edited pigs and a population of equivalent non-edited pigs when exposed to PRRSV of the same virulence level (ideally the same isolate) demonstrates improved tolerance or resistance. Improved tolerance or resistance can be demonstrated by a reduced susceptibility to PRRSV inflection, or a lessening of the symptoms when infection occurs. Improved resistance to infection in a swine can be tested in vitro using the methodologies described below for PAM and PMM cells.

“Protein” and “peptide”, as used herein, can be used interchangeably (unless the context suggests otherwise) and mean at least two covalently attached amino acids linked by a peptidyl bond. The term protein encompasses purified natural products, or products which may be produced partially or wholly using recombinant or synthetic techniques. The terms peptide and protein may refer to an aggregate of a protein such as a dimer or other multimer, a fusion protein, a protein variant, or derivative thereof. A protein may comprise amino acids not encoded by a nucleic acid codon, i.e. non-natural amino acids.

### INTRODUCTION

PRRS is one of the most economically important infectious diseases affecting pigs worldwide. The “mystery swine disease” was first observed almost simultaneously in North America and in Europe in the late 1980s [1,2]. The causative agent of PRRS was identified to be a virus later named PRRS virus (PRRSV). Infected pigs may present with symptoms involving inappetence, fever, lethargy, and respiratory distress. However, the most devastating effects of PRRSV infection are observed in young piglets and pregnant sows. In pregnant sows an infection with PRRSV can cause a partial displacement of the placenta, leading to full abortions or to death and mummification of fetuses in utero [3]. Late-term abortions occur in up to 30% of infected sows with litters containing up to 100% stillborn piglets. Live-born piglets from an antenatal infection are often weak and display severe respiratory symptoms, with up to 80% of them dying on a weekly basis pre-weaning [4,5]. Young piglets infected with PRRSV often display diarrhea and severe respiratory distress caused by lesions in the lung. In pre-weaned piglets the infection may be transmitted via the mammary gland secretions of an infected sow [6]. At this age the infection has a fatal outcome in up to 80% of animals. After weaning mortality rates reduce, but continued economic losses due to reduced daily gain and feed efficiency are often observed [4,7,8]. Due to reduction or loss of pregnancies, death in young piglets, and decreased growth rates in all PRRSV infected pigs it is estimated that more than $650m are lost annually to pork producers in the United States alone [9,10].

PRRSV is an enveloped, plus-strand RNA virus belonging to the Arteriviridae family in the order Nidovirales [11,12]. The PRRSV genome (˜15 kb) encodes at least 12 non-structural and seven structural proteins. The viral RNA is packaged by the nucleocapsid protein N, which is surrounded by the lipoprotein envelope, containing the non-glycosylated membrane proteins M and E, as well as four glycosylated glycoproteins GP2, GP3, GP4, and GP5, whereby GP2, 3, and 4 form a complex [13-17].

PRRSV has a very narrow host range, infecting only specific subsets of porcine macrophages [18-20]. It is unknown yet how widespread PRRSV infections are within the superfamily of the Suidae. Whereby European wild boars have been shown to act as a reservoir for PRRSV [21], little is known about infection in African suids, such as bushpigs and warthogs. In vitro virus replication is supported by the African Green Monkey cell line MARC-145. Entry of PRRSV into macrophages has been shown to occur via pH-dependent, receptor mediated endocytosis [22,23]. Various attachment factors and receptors have been indicated to be involved in the PRRSV entry process (reviewed in [24]). Heparan sulphate was identified early as an attachment factor of the virus [25-27]. In vitro infection of pulmonary alveolar macrophages (PAMs) but not MARC-145 cells was shown to be inhibited by an antibody targeting CD169 (sialoadhesin), a lectin expressed on the surface of macrophages [28]. Overexpression of CD169 in previously non-permissive PK-15 cells showed internalization but not productive replication of PRRSV [29]. Finally, an in vivo challenge of genetically modified pigs in which the CD169 gene had been knocked out revealed no increased resistance to PRRSV infection, suggesting that CD169 is an attachment factor that is not essential for PRRSV infection [30]. Even though cell surface protein expression is a major determinant of PRRSV binding and internalization, there appears to be a redundancy amongst cell surface attachment factors, with the potential for additional, as yet unidentified receptors, being involved [31]. The scavenger receptor CD163, also known as haptoglobin scavenger receptor or p155, is expressed on specific subtypes of macrophages and has been identified as a fusion receptor for PRRSV. The extracellular portion of CD163 forms a pearl-on-a-string structure of nine scavenger receptor cysteine-rich (SRCR) domains and is anchored by a single transmembrane segment and a short cytoplasmic domain [32]. CD163 has a variety of biological functions, including mediating systemic inflammation and the removal of hemoglobin from blood plasma (reviewed in [33,34]). Overexpression of CD163 renders non-susceptible cells permissive to PRRSV infection [35], whereby it was found that CD163 does not mediate internalization but is crucial for fusion [36]. The transmembrane anchoring and an interaction with the SRCR domain 5 (SRCR5) of CD163 were found to be essential for successful infection with PRRSV [34,35]. Recent in vivo experiments with CD163 knock-out pigs have been performed [37]. However, as CD163 has important biological functions the complete knockout could have a negative physiological impact pigs, particularly with respect to inflammation and/or infection by other pathogens.

This study aimed to generate pigs with a defined CD163 SRCR5 deletion and to assess the susceptibility of macrophages from these pigs to PRRSV infection.

Materials and Methods

All animal work was approved under UK Home Office license after review by the University of Edinburgh's Animal Ethics Committee and was carried out in accordance with the approved guidelines.

Cells and Viruses

Primary pulmonary alveolar macrophages (PAMs) for the propagation of PRRSV genotype 1, subtype 1 strain H2 (PRRSV H2) [52], subtype 2 strain DAI (PRRSV DAI) [53], and subtype 3 strain SU1-Bel (PRRSV SU1-Bel)[54] were harvested from wild type surplus research animals aged 6-9 weeks as previously described [45]. Briefly, animals were euthanized according to a schedule I method. Lungs were removed and transferred on ice to a sterile environment. PAMs were extracted from lungs by washing the lungs twice with warm PBS, massaging to release macrophages. Cells were collected by centrifugation for 10 min at 400 g. When necessary red cells were removed using red cell lysis buffer (10 mM KHCO₃, 155 mM NH₄Cl, 0.1 mM EDTA, pH 8.0) for 5 min before washing again with PBS. Cells were collected by centrifugation as before and frozen in 90% FBS (HI, GE Healthcare), 10% DMSO (Sigma). Cells were frozen gradually at 1° C./min in a −80° C. freezer before being transferred to −150° C.

PAMs from the animals 627, 628, 629, 630, 633, and 634 were collected at 8 weeks of age. For this the piglets were sedated using a Ketamine/Azaperone pre-medication mix and anaesthetized with Ketamine/Midazolam. Anesthesia throughout the procedure was maintained using Sevoflurane. PAMs were collected by bronchoalveolar lavage (BAL) through an intubation with an air flow access. Three lung segments were flushed in each animal using 2×20 ml PBS. Fluid recovery was between 60-80%. Cells were collected by centrifugation for 10 min at 400 g from the BAL fluid and frozen as above.

Peripheral blood monocytes (PBMCs) were isolated as described previously [45]. Briefly, blood was collected using EDTA coated vacuum tubes from the jugular vein of the piglets at 10 weeks of age. Blood was centrifuged at 1200 g for 15 min and buffy coat transferred to PBS. Lymphoprep (Axis-Shield) was overlaid with an equal volume of buffy coat/PBS and centrifuged for 45 min at 400 g. The mononuclear cell fraction was washed with PBS, cells collected and frozen as described above.

PAM cells were cultivated in RPMI-1640, Glutamax (Invitrogen), 10% FBS (HI, GE Healthcare), 100 IU/ml penicillin and 100 μg/ml streptomycin (Invitrogen) (cRPMI). PBMCs were cultivated in cRPMI supplemented with rhCSF-1 (1×10⁴ units/ml; a gift from Chiron) for 6 days prior to infection.

PK15 cells were cultured in DMEM supplemented with Glutamax (Invitrogen), 10% FBS (HI, GE Healthcare), 100 IU/ml penicillin and 100 μg/ml streptomycin (Invitrogen).

Design and In Vitro Cutting Efficiency Assessment of Guide RNAs

Three potential guide RNA sequences were selected in the 200 bp of intron 6 and one in the 97 bp long intron 7. Oligomers (Invitrogen) were ordered, hybridized as previously described [72] then ligated into the BbsI sites of plasmid pSL66 (a derivative of px458 with modifications to the sgRNA scaffold as described by [42]). The generated plasmids contain a hU6 promoter driving expression of the guide RNA sequence and a CBA promoter driving Cas9-2A-GFP with an SV40 nuclear localization signal (NLS) at the N-terminus and a nucleoplasmin NLS at the C-terminus of Cas9. Cutting efficiency of each guide was assessed by transfection of the plasmids into pig PK15 cells using a Neon transfection system (Invitrogen) set at 1400 mV with 2 pulses of 20 mS. 48 hours post-transfection GFP positive cells were collected using a FACS Aria III cell sorter (Becton Dickinson) and cultured for a further 4 days prior to preparation of genomic DNA (DNeasy Blood & Tissues Kit, Qiagen). PCR across the target sites was with oSL46 (ACCTTGATGATTGCGCTCTT—SEQ ID NO:17) and oSL47 (TGTCCCAGTGAGAGTTGCAG—SEQ ID NO:18) using AccuPrime Taq DNA polymerase HiFi (Life Technologies) to produce a product of 940 bp. A Cell assay (Transgenomic; Surveyor Mutation Detection Kit) was performed as previously described [73]. Co-transfection of PK15 cells with pairs of plasmids encoding guides flanking exon 7 were carried out as described above with the exception that cells were harvested at 40 hours post-transfection without enrichment for GFP expression. In this instance a truncated PCR product was observed in addition to the 940 bp fragment, indicating deletion of exon 7.

Based on both single and double cutting efficiencies guide RNAs SL26 (GAATCGGCTAAGCCCACTGT—SEQ ID NO:7), located 121 bp upstream of exon 7, and SL28 (CCCATGCCATGAAGAGGGTA—SEQ ID NO:11), located 30 bp downstream of exon 7 were selected for in vivo experiments.

Generation of Guide RNA and Quality Assessment

A DNA oligomer fragment containing the entire guide RNA scaffold and a T7 promoter was generated by PCR from the respective plasmid template as follows; a forward primer containing the T7 promoter followed by the first 18 bp of the respective guide RNA and the reverse primers oSL6 (AAAAGCACCGACTCGGTGCC—SEQ ID NO:19) were used in combination with the Phusion polymerase (NEB). DNA fragments were purified on a 1.5% agarose gel using the MinElute Gel Extraction Kit (Qiagen) according to the manufacturer's instructions. DNA eluate was further treated with 200 μg/ml Proteinase K (Qiagen) in 10 mM Tris-HCl pH 8.0, 0.5% SDS for 30 min at 50° C. followed by phenol/chloroform extraction. Guide RNAs were generated from the resultant DNA fragment using the MEGAshortscript Kit (Thermo Fisher) according to the manufacturer's instructions. RNA was purified using phenol/chloroform extraction followed by ethanol precipitation and resuspended in EmbryoMax Injection Buffer (Millipore). Purity and concentration of the RNA was assessed using an RNA Screen Tape (Agilent) on an Agilent TapeStation according to the manufacturer's instructions.

Zygote Injection and Transfers

Embryos were produced from Large White gilts as described previously [73]. Briefly, gilts were superovulated using a regumate/PMSG/Chorulon regime between day 11 and 15 following estrus. Following heat, the donor gilts were inseminated twice in a 6 hour interval. Zygotes were surgically recovered from mated donors into NCSU-23 HEPES base medium, then subjected to a single 2-5 pl cytoplasmic injection with an injection mix containing 50 ng/μl of each guide (SL26 and SL28) and 100 ng/μl Cas9 mRNA (PNA Bio or Tri-Link) in EmbryoMax Injection buffer (Millipore). Recipient females were treated identically to donor gilts but remained unmated. During surgery, the reproductive tract was exposed and 24-39 zygotes were transferred into the oviduct of recipients using a 3.5 French gauge tomcat catheter. Litter sizes ranged from 5-12 piglets.

In Vitro Assessment Genome Editing in Blastocyst

Uninjected control zygotes and injected surplus zygotes are cultivated in NCSU-23 HEPES base medium, supplemented with cysteine and BSA at 38.5° C. for 5-7 days. Blastocysts were harvested at day 7 post cultivation and the genome amplified using the REPLI-g Mini Kit (Qiagen), according to the manufacturer's instructions. Genotyping was performed as described below.

Genotyping

Genomic DNA was extracted from ear biopsy or tail clippings taken from piglets at 2 days postpartum using the DNeasy Blood and Tissue Kit (Qiagen). The region spanning intron 6 to exon 8 was amplified using primers oSL46 (ACCTTGATGATTGCGCTCTT—SEQ ID NO:17) and oSL47 (TGTCCCAGTGAGAGTTGCAG—SEQ ID NO:18), generating a 904 bp product from the intact allele and a 454 bp product if complete deletion of exon 7 had occurred. PCR products were analyzed by separation on a 1% agarose gel and subsequent Sanger sequencing of all truncated fragments. Fragments corresponding to the wild type length were further analyzed by T7 endonuclease I (NEB) digestion according to the manufacturer's instructions.

RNA Phenotyping

RNA was isolated from 1E6 PAM cells, isolated by BAL as described above, using the RNeasy Mini Kit (Qiagen), according to the manufacturer's instructions, including an on-column DNase digestion. First-strand cDNA was synthesized using an Oligo-dT primer in combination with SuperScript II reverse transcriptase (Invitrogen), according to the manufacturer's instructions. The cDNA was used to assess the RNA phenotype across exons 4 to 9 using primers P0083 (ATGGATCTGATTTAGAGATGAGGC—SEQ ID NO:20) and P0084 (CTATGCAGGCAACACCATTTTCT—SEQ ID NO:21), resulting in a PCR product of 1686 bp length for the intact allele and 1371 bp following precise deletion of exon 7. PCR products were analyzed by separation on a 1% agarose gel and subsequent Sanger sequencing of deletion fragments.

Protein Phenotype Analysis by Western Blotting

4E5 PAM cells isolated by BAL were collected by centrifugation at 300 rcf for 10 min. The pellet was resuspended in Laemmli sample buffer containing 100 mM DTT, boiled for 10 min at 95° C. and subjected to electrophoresis on 7.5% acrylamide (Bio-Rad) gels. After transfer to a nitrocellulose membrane (Amersham), the presence of cellular proteins was probed with antibodies against CD163 (rabbit pAb, abcam, ab87099) at 1 μg/ml, and β-actin (HRP-tagged, mouse mAb, Sigma, A3854) at 1:2000. For CD163 the blot was subsequently incubated with HRP-labelled rabbit anti-mouse antibody (DAKO, P0260) at 1:5000. Binding of HRP-labelled antibodies was visualized using the Pierce ECL Western Blotting Substrate (Thermo Fisher), according to the manufacturer's instructions.

Quantification of CD163 mRNA by RT-qPCR

RNA was isolated from 1E6 PAMs using the RNeasy Mini Kit (Qiagen), according to the manufacturer's instructions, including an on-column DNase digestion. RNA levels were measured using the GoTaq 1-Step RT-qPCR system (Promega) according to the manufacturers' instructions on a LightCycler 480 (Roche). mRNA levels of CD163 were quantified using primers P0074 (CATGGACACGAGTCTGCTCT—SEQ ID NO:22) and P0075 (GCTGCCTCCACCTTTAAGTC—SEQ ID NO:23) and reference mRNA levels of β-actin using primers P0081 (CCCTGGAGAAGAGCTACGAG—SEQ ID NO:24) and P0082 (AAGGTAGTTTCGTGGATGCC—SEQ ID NO:25).

Characterization of Macrophages by Flow Cytometry

PAMs were seeded one day prior to analysis. PBMCs were seeded seven days prior to analysis and differentiated by CSF1 stimulation to yield PBMC-derived macrophages (PMMs). Cells were harvested by scraping with a rubber policeman and fixed in 4% formaldehyde/PBS for 15 min at room temperature. Cells were incubated with blocking solution (PBS, 3% BSA) for 45 min before staining with antibodies. Cells were stained with antibodies targeting either mouse anti-pig CD14 (AbD Serotec, MGA1273F, 1:50) and mouse anti-pig CD16 (AbD Serotec, MCA2311PE, 1:200), mouse anti-pig CD169 (AbD Serotec, MCA2316F, 1:50) and mouse anti-pig CD172a (SoutherBiotech, 4525-09, 1:400), mouse anti-human CD151 (AbD Serotec, MCA1856PE, 1:50) and mouse anti-pig SWC9 (CD203a) (AbD Serotec, MCA1973F, 1:50), mouse anti-pig CD163 (AbD Serotec, MCA2311PE, 1:50), or mouse IgG1 or an IgG2b negative control (AbD Serotec, MCA928PE, MCA691F, or Sigma, F6397; same concentration as primary Ab). The cells were washed three times with PBS and resuspended in FACS buffer (2% FBS, 0.05M EDTA, 0.2% NaN₃ in PBS). Gene expression determined by antibody labelling was assessed by FACS analysis on a FACS Calibur (Becton Dickinson) using FlowJo software.

High MOI Single-Round Infection Assay

PAMs were seeded one day prior to infection. PBMCs were seeded seven days prior to infection and differentiated by CSF1 stimulation to yield PBMC-derived macrophages PMMs. Cells were inoculated at MOI=1 of the respective virus strain (PRRSV H2, DAI, or SU1-Bel) in cRPMI for 3 h at 37° C. The inoculum was replaced by warm cRPMI. At 19 hpi cells were detached by using a cell scraper. Cells were fixed in 4% Formaldehyde (Sigma-Aldrich) in PBS (Gibco) for 15 min at RT, washed with PBS, and subsequently permeabilized in PBS containing 0.1% Triton-X-100 (Alfa Aesar) for 10 min. Cells were incubated with antibody against PRRSV-N (SDOW17-F, RTI, KSL0607, 1:200) and CD163 (AbD Serotec, MCA2311PE, 1:50) or mouse IgG1 negative controls, as described above, in 3% BSA in PBS. The cells were washed three times with PBS and re-suspended in FACS buffer. Infection levels, determined by antibody labelling, were assessed by FACS analysis on a FACS Calibur (Benson Dickson) using FlowJo software.

Low MOI Multiple-Round Infection Assay

PAMs were seeded one day prior to infection. PBMCs were seeded seven days prior to infection and differentiated by rhCSF1 stimulation to yield PMMs. Cells were inoculated at MOI=0.1 with the respective virus strain (PRRSV H2, DAI, or SU1-Bel) in cRPMI for 3 h at 37° C. Inoculum was removed, cells washed 1× with PBS, and infection continued. At the indicated times post inoculation samples were harvested to be assessed. All samples were frozen and processed once all samples from a time course had been collected.

Viral RNA (vRNA) was extracted from the supernatant samples using the QIAmp Viral RNA Mini Kit according to the manufacturer's instructions. The viral RNA levels were quantified by RT-qPCR using the GoTaq Probe 1-Step RT-qPCR system (Promega) for PRRSV H2 and SU1-Bel and the GoTaq 1-Step RT-qPCR system (Promega) for PRRSV DAI, according to the manufacturer's instructions. For this the following primers and probes were used: H2 fwd (GATGACRTCCGGCAYC—SEQ ID NO:26), H2 rev (CAGTTCCTGCGCCTTGAT—SEQ ID NO:27), H2 probe (6-FAM-TGCAATCGATCCAGACGGCTT-TAMRA—SEQ ID NO:28), (optimal H2 primer/probe sequences obtained from JP Frossard, AHVLA), SU1-Bel fwd (TCTTTGTTTGCAATCGATCC—SEQ ID NO:29), SU1-Bel rev (GGCGCACTGTATGACTGACT—SEQ ID NO:30), SU1-Bel probe (6-FAM-CCGGAACTGCGCTTTCA-TAMRA—SEQ ID NO:31), DAI fwd (GGATACTATCACGGGCGGTA—SEQ ID NO:32), DAI rev (GGCACGCCATACAATTCTTA—SEQ ID NO:33). RNA levels were measured on a LightCycler 480 (Roche) using a standard curve generated from vRNA isolates of high titer stocks.

Infectivity of the virus produced was assessed using a TCID₅₀ assay of selected time points on PAMs isolated from wild type surplus research animals.

mRNA and Protein Levels of Heme Oxygenase 1 Upon Hb-Hp Stimulation of PMMs

PBMCs were seeded seven days prior to analysis and differentiated by CSF1 stimulation to yield PMMs. Hemoglobin (Hb, Sigma-Aldrich, AO, H0267) and Haptoglobin (Hp, Sigma Aldrich, Phenotype 2-2, H9762) were mixed in a 1:1 wt/wt ratio in PBS for 15 min on a vertical roller before experimentation. PMMs were incubated with 100 μg/ml Hb-Hp in cRPMI for 24 h at 37° C. Cells were harvested by scraping with a rubber policeman. RNA was isolated from 1E6 cells using the RNeasy Mini Kit (Qiagen), according to the manufacturer's instructions, including an on-column DNase digestion. RNA levels were measured using the GoTaq 1-Step RT-qPCR system (Promega) according to the manufacturers' instructions on a LightCycler 480 (Roche). mRNA levels of heme oxygenase 1 (HO-1) were quantified using primers P0239 (TACATGGGTGACCTGTCTGG—SEQ ID NO:34) and P0240 (ACAGCTGCTTGAACTTGGTG—SEQ ID NO:35) and reference mRNA levels of β-actin using primers P0081 and P0082. For analysis of protein levels of HO-1 cells were collected by centrifugation at 300 rcf for 10 min. The pellet was re-suspended in Laemmli sample buffer containing 100 mM DTT, boiled for 10 min at 95° C. and subjected to electrophoresis on 12% acrylamide (Bio-Rad) gels. After transfer to a nitrocellulose membrane (Amersham), the presence of cellular proteins was probed with antibodies against HO-1 (mouse mAb, abcam, ab13248, 1:250), and calmodulin (rabbit mAb, abcam, ab45689, 1:1000). The blot was subsequently incubated with HRP-labelled goat anti-rabbit antibody (DAKO, PI-1000) at 1:5000. Binding of HRP-labelled antibodies was visualized using the Pierce ECL Western Blotting Substrate (Thermo Fisher), according to the manufacturer's instructions.

Quantification and Visualization of Hemoglobin-Haptoglobin Uptake

PBMCs were seeded seven days prior to analysis and differentiated by CSF1 stimulation to yield PMMs. For fluorescence microscopy, cells were seeded on glass cover slips. Hemoglobin (Sigma-Aldrich, AO, H0267) was labeled with Alexa Fluor 488 (AF-488) using a protein labelling kit (Molecular Probes) according to the manufacturer's instructions. Hb_{AF488} and Hp were mixed in a 1:1 wt/wt ratio in PBS for 15 min on a vertical roller before experimentation. PMMs were incubated with 10 μg/ml Hb_{AF488}-Hp in cRPMI for 30 min at 37° C.

For quantification by FACS the cells were collected with a rubber policeman and washed three times with Ca²⁺/Mg²⁺-free PBS to remove surface bound Hb_{AF488}-Hp as described previously [60]. Cells were fixed in 4% (wt/v) formaldehyde (Sigma-Aldrich) in PBS (Gibco) for 15 min at RT, washed with PBS, and subsequently permeabilized in PBS containing 0.1% Triton-X-100 (Alfa Aesar) for 10 min. Cells were stained with mouse anti pig CD163 antibody (AbD Serotec, MCA2311PE, 1:50) as described above then washed three times with PBS and re-suspended in FACS buffer. Gene expression determined by antibody labelling was assessed by analysis on a FACS Calibur (Becton Dickinson) using FlowJo software.

For immunofluorescence imaging cells were washed three times with Ca²⁺/Me-free PBS and fixed in 4% formaldehyde (Sigma-Aldrich) in PBS (Gibco) for 15 min at RT, washed with PBS, then permeabilized in PBS containing 0.1% Triton-X-100 (Alfa Aesar) for 10 min. Cells were washed with PBS and incubated with antibody against CD163 (rabbit pAb, abcam, ab87099, 5 μg/ml) in blocking buffer (PBS, 3% FBS) for 1 h, washed, and incubated with secondary goat anti-rabbit AF594 antibody (A11037, 1:100), AF647 phalloidin (A22287, 1:100), and DAPI (1:10,000; all Life Technologies). The samples were analyzed using a confocal laser-scanning microscope (Zeiss LSM-710).

Immunofluorescence Analysis of RTC Formation in Infected PAMs

PAMs were seeded onto coverslips one day prior to infection. Cells were inoculated at MOI=2 of the respective virus strain (PRRSV H2, DAI, or SU1-Bel) in cRPMI for 3 h at 37° C. The inoculum was replaced by warm cRPMI. At 19 hpi cells were fixed in 4% formaldehyde (Sigma-Aldrich) in PBS (Gibco) for 15 min at RT, washed with PBS, and permeabilized as described above. Cells were washed with PBS and incubated with antibody against PRRSV nsp2 (A gift from Ying Fang, South Dakota State University, [74], 1:400) in blocking buffer for 1 h, washed, and incubated with secondary goat anti-mouse AF488 antibody (A11029, 1:100), AF568 phalloidin (A12380, 1:100), and DAPI (1:10,000; all Life Technologies). The samples were analyzed using a confocal laser-scanning microscope (Zeiss LSM-710).

Results

Generation of Live CD163 SRCR5 Deletion Pigs by CRISPR/Cas9 Editing in Zygotes

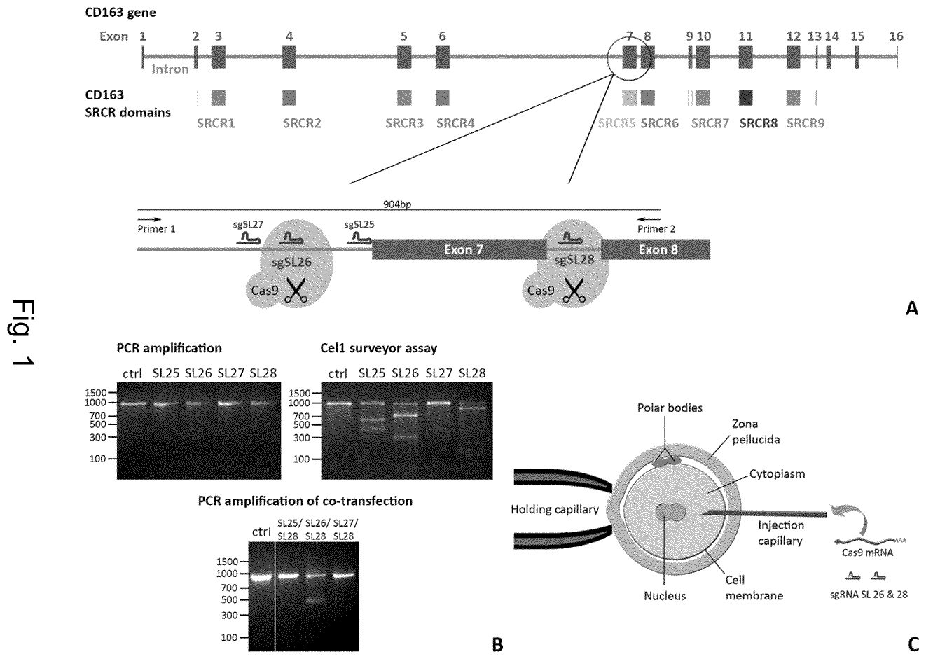

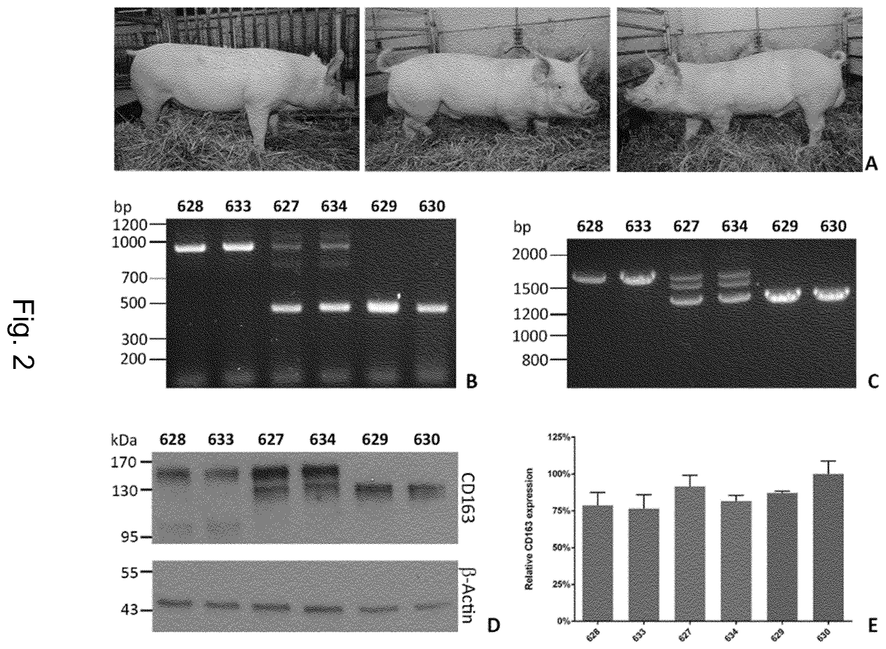

The CD163 gene is not correctly represented in the current pig reference genome sequence (Sscrofa10.2) [38]. Through targeted sequencing we have established a detailed model of the porcine CD163 locus (unpublished results L. Zen/A. Archibald/T. Ait-Ali)—the genomic sequence of the CD163 gene is set out below as SEQ ID NO:1. Briefly, CD163 is encoded by 16 exons with exons 2-13 predicted to encode the SRCR domains of the protein [39]. Interestingly, SRCR5 is predicted to be encoded by one single exon, namely exon 7 (FIG. 1A). Thus, an editing strategy was developed to excise exon 7 using the CRISPR/Cas9 genome editing system [40,41]. A combination of two guide RNAs, one located in the intron 5′ to exon 7 and one in the short intron between exons 7 and 8 was predicted to generate a deletion of exon 7, whilst allowing appropriate splicing of the remaining exons. Due to the short length of the intron between exons 7 and 8 (97 bp) only one suitably unique targeting sequence (crRNA) with a corresponding protospacer adjacent motif was identified. Three candidate crRNA sequences were selected in the immediate upstream area of exon 7. It should be noted that alternative site-specific nucleases (ZFNs or TALENs, for example) could also be used, and the skilled person could readily determine suitable target sites; notably these editors do not require the presence of the PAM sequence, and thus there is less limitation on target site selection.

All four sequences were assessed in vitro for cutting efficiency by transfection of porcine kidney PK15 cells with a plasmid based on px458 [42] encoding the complete single guide sequence (sgRNA), driven by the hU6 promoter, and a CAG promoter driving NLS-Cas9-2A-GFP. Transfected cells were isolated by fluorescence activated cell sorting (FACS) for GFP and cutting efficiency at the target site was assessed using a Cell surveyor assay. Three out of four guides were shown to direct cutting of DNA as anticipated (2 upstream and one downstream of exon 7). Following double transfection assay and subsequent PCR analysis it was found that the combination of guides SL26 and 51_28 effectively generated the exon 7 deletion in the CD163 gene (FIG. 1B). Based on these results the guide combination of sgSL26 and sgSL28 was used for in vivo experiments.

sgRNAs SL26 and SL28 were microinjected together with mRNA encoding the Cas9 nuclease into the cytosol of zygotes. Editing efficiency was assessed in a small number of injected zygotes by in vitro culture to the blastocyst stage, genomic DNA extraction, whole genome amplification and PCR amplification across exon 7. The analysis revealed that two out of 17 blastocysts contained a deletion of the intended size and Sanger sequencing confirmed the deletion of exon 7. Edited blastocyst B2 showed a clean deletion and subsequent re-ligation at the cutting sites of sgSL26 and sgSL28, whilst edited blastocyst B14 showed that in addition to the intended deletion there was also a random insertion of 25 nucleotides at the target site. None of the full length PCR products showed nucleotide mismatches at either cutting site in a T7 endonuclease assay. The editing rate in the blastocysts corresponds to an overall editing rate of 11.7%.

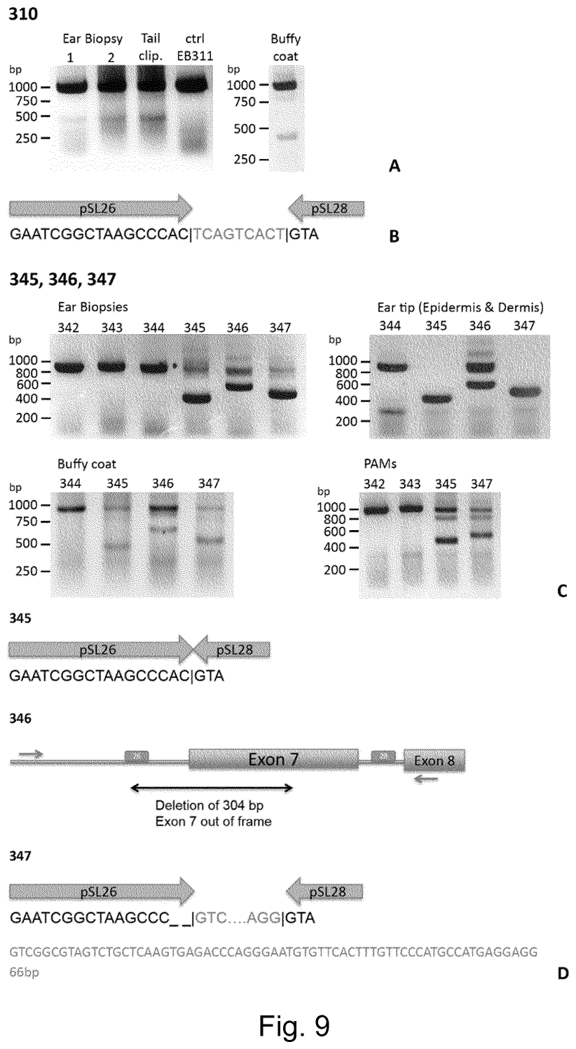

To generate live pigs, 24-39 zygotes injected with sgSL26, sgSL28, and Cas9 mRNA were transferred into the oviduct of recipient gilts. A total of 32 live piglets were born and genotyping of ear and tail biopsies revealed that four of the piglets had an exon 7 deletion, corresponding to 12.5% of the total. In addition to the intended deletion of exon 7, three out of the four animals showed insertion of new DNA at the target site probably as a consequence of non-homologous end joining repair. Pig 347 showed a 2 bp truncation at the sgSL26 cutting site and a 66 bp insertion between the cutting sites, pig 346 showed a deletion of 304 bp after the cutting site of sgSL26, and pig 310 showed a short 9 bp insertion (having the sequence TCAGTCACT) at the cutting sites. Pig 345 was found to have a precise deletion of exon 7 without insertion or deletion of random nucleotides at the cut sites (FIGS. 9, B and D). Interestingly, PCR amplification indicated that pigs 310, 345, and 347 were all mosaic for the editing event, with pig 310 having a low frequency heterozygous (one allele edited) compared to unedited cells, whilst in pigs 345 and 347 have both homozygous (both alleles edited) and heterozygous cell types (FIGS. 9, A and C).

Genotype and Phenotype of F1 Generation Pigs

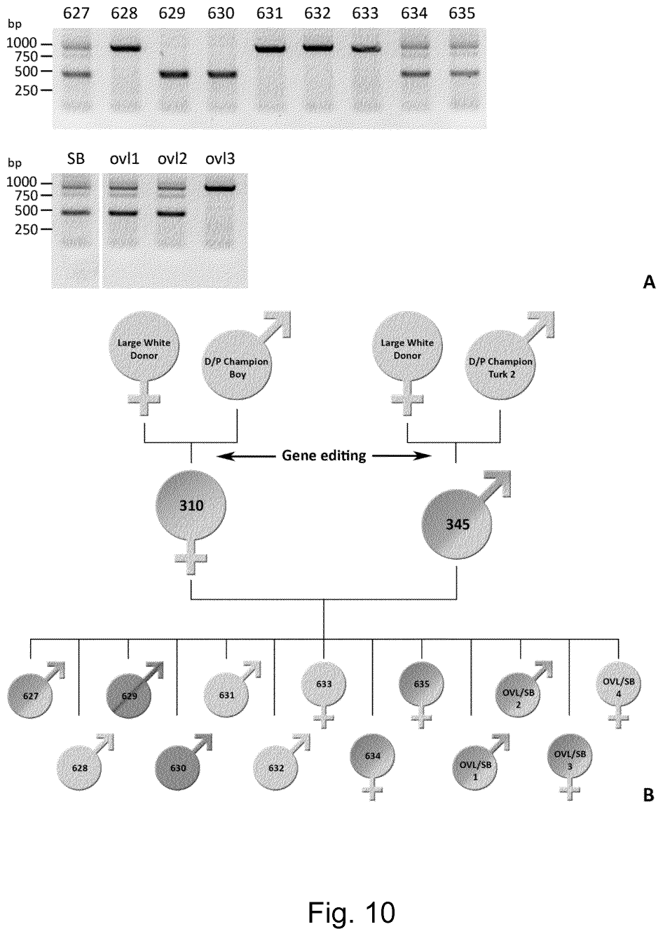

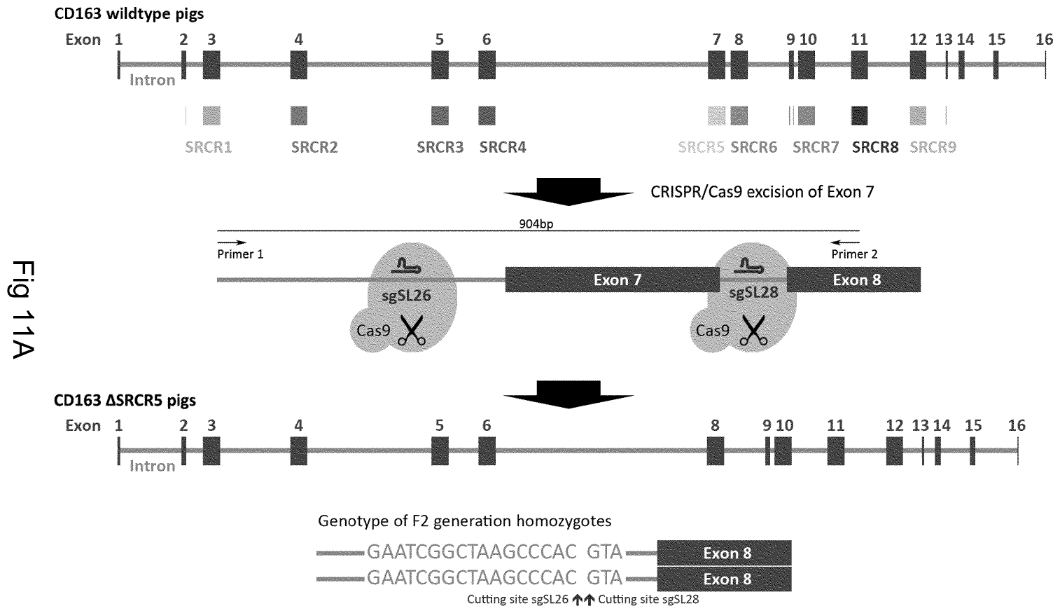

To generate fully homozygous and heterozygous pigs, 310 was mated with 345. This mating yielded a litter of 6 heterozygous, 2 biallelic/homozygous CD163 SRCR5 deletion (ΔSRCR5), and 4 wild type CD163 piglets (FIG. 10). Sequencing of the animals revealed all the heterozygotes to have inherited their edited allele from 345. Pig 629 was found to be biallelic for the exon 7 deletion with one allele carrying the genotype of 345 and the other allele the one from 310. Interestingly 630 was found to be homozygous for the edited allele with the 9 bp insert between the cutting sites of sgSL26 and sgSL28 as found in the 310 founder/parent (Table 1). We conclude that this homozygous state has arisen from a gene conversion event in the zygote.

TABLE 1
Genotypes and growth of assessed F1 animals.

| Animal ID | Gender | Birth weight | 60 day weight | Type |
|---|---|---|---|---|
| 628 | male | 1.2 kg | 25 kg | wild type |
| 633 | female | 1.6 kg | 26 kg | wild type |
| 627 | male | 1.6 kg | 25 kg | heterozygous |
| 634 | female | 1.3 kg | 27 kg | heterozygous |
| 629 | male | 1.4 kg | 25 kg | biallelic |
| 630 | male | 1.6 kg | 27 kg | homozygous |

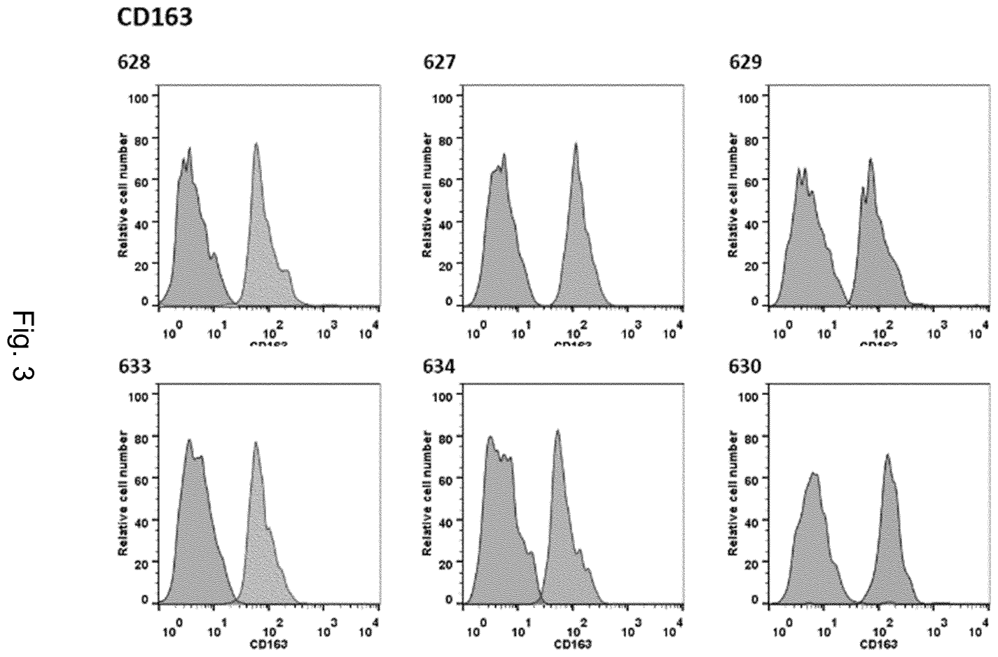

Animals 627, 628, 629, 630, 633, and 634 were selected for further analysis, representing the various genotypes (wild type, heterozygous, and biallelic/homozygous) and genders. Growth rates of both ΔSRCR5 and heterozygous animals were comparable to wild type animals (Table 1). Blood samples were taken from all six animals at 10 weeks of age and analyzed by a full blood count conducted by the diagnostics laboratory at the Royal (Dick) School of Veterinary Studies, University of Edinburgh. The blood counts of all animals were within reference values (Table 1). Size, stature and other morphological features of ΔSRCR5 and heterozygous pigs were comparable to their wild type siblings (FIG. 2A).

At 8 weeks of age, pulmonary alveolar macrophages (PAMs) were isolated from all six animals by bronchoalveolar lavage (BAL). DNA was extracted from the PAMs and analyzed by PCR and Sanger sequencing. The PAM genotype confirmed the results obtained from the ear biopsies; 628 and 633 were wild type, 627 and 633 heterozygous, and 629 and 630 ΔSRCR5, respectively. Sequencing of PCR products confirmed that all editing events had resulted in complete deletion of exon 7. Whilst pigs 627 and 633 had a clean deletion of exon 7 with precise re-ligation at the sgSL26 and sgSL28 cutting sites in one allele, 629 had one allele with a clean deletion and one allele with a 9 bp insertion between the sites, and pig 630 had both alleles with the 9 bp insertion. RNA was extracted from the PAMs, converted into cDNA using oligo(dT) primed reverse transcription, amplified by PCR and analyzed by Sanger sequencing. PCR products spanning exons 4 to 9 showed the expected 315 bp deletion in both heterozygous and ΔSRCR5 animals (FIG. 2C). A third fragment situated between the full length and exon 7 deletion band in 627 and 634 was confirmed to be a hybrid of the full length and the exon 7 deletion fragment. This shows that deletion of exon 7 has not disrupted the use of the correct splice acceptor site of exon 8. Expression of CD163 protein was assessed by western blot of PAM lysate. The wild type pigs 628 and 633 expressed the full length protein with a predicted size of 120 kDa but is described to run at roughly 150 kDa [43], likely due to glycosylation, whereby a protein band at roughly 100 kDa may indicate the expression of another isoform, which could correspond to the described human isoform CRA_a or CRA_b (GenBank references EAW88664.1 and EAW88666.1). Heterozygous animals 627 and 634 express both the full-length and the ΔSRCR5 protein (FIG. 2D). The band of the full-length protein is clearly stronger, indicating either higher expression of the full-length gene or increased binding of the full-length protein by the polyclonal CD163 antibody used in this study. To further examine this, gene expression was quantified by RT-qPCR on RNA extracted from PAMs and normalized to β-actin expression, demonstrating no significant difference in total CD163 mRNA expression between wild type, heterozygous and ΔSRCR5 animals (FIG. 2E).

Pulmonary Alveolar Macrophages of ΔSRCR5 Pigs are Fully Differentiated and Express Macrophage-Specific Surface Proteins

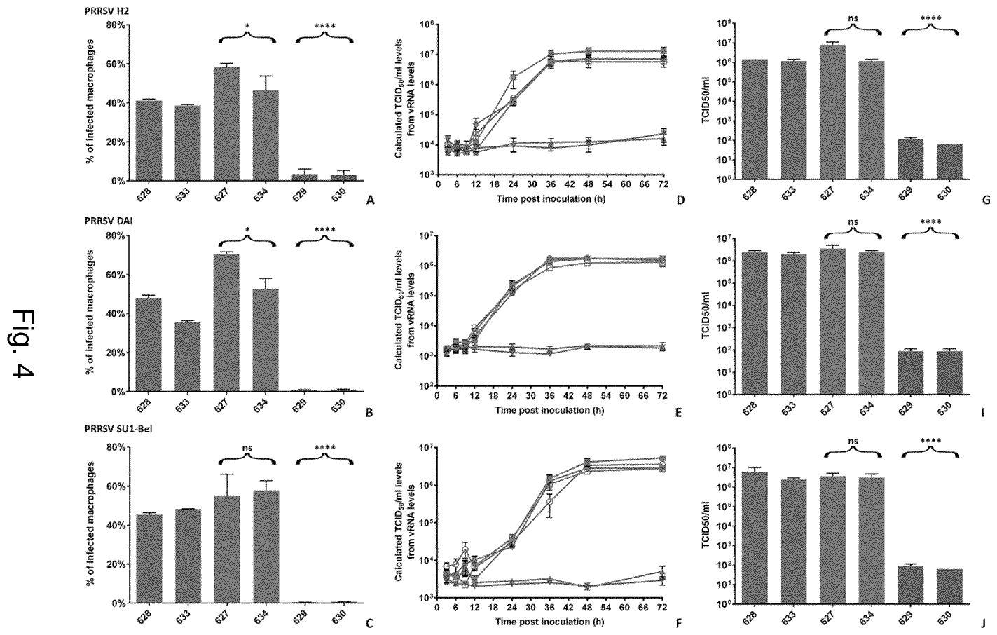

PAMs isolated by BAL were characterized for the expression of macrophage-specific surface proteins. CD14 and CD16 are not expressed on monocytes but levels increase upon maturation into macrophages. In PAMs CD14 is found at moderate levels, whilst CD16 is strongly expressed [44]. CD14/CD16 staining of the PAMs from the ΔSRCR5, heterozygous, and wild type animals were all within the previously observed and documented levels [45], with difference being observed between the various genotypes (data not shown). CD172a, or also known as SIRPα, is expressed at high levels on both monocytes and macrophages [46] and was expressed at high levels in cells from all animals. CD169, described as an attachment factor for PRRSV [29], is not expressed in monocytes but is highly expressed in tissue macrophages [47] and was expressed at expected levels in cells from our animals (data not shown). As in humans, expression of CD163 in pigs is restricted to monocytes and macrophages. CD163 is expressed at high levels in tissue macrophages, but at low levels in blood monocytes and in bone marrow-derived macrophages [48] (porcine macrophage markers are reviewed in [49]). Both the wild type and the SRCR5 deletion CD163 were recognized on the surface of the PAMs (FIG. 3). This indicates that the SRCR5 deleted version of Cd163 is likely to be properly folded as the clone 2A10/11 antibody only recognizes the protein in a non-reduced, native conformation. The medians of CD163 fluorescence intensity of pigs 628, 633, 627, 634, 629, 630 were 35.9, 22.7, 26.4, 24.4, 17.9, and 26.7, respectively, with isotype control medians ranging from 2.13-3.84. Overall, PAMs isolated from all animals, independent of their genotype were shown to be fully differentiated and to express macrophage-specific surface markers, including CD169 and CD163, which have implicated functions in PRRSV entry.

ΔSRCR5 Pulmonary Alveolar Macrophages are not Susceptible to Infection with PRRSV Genotype 1

PRRSV has two different genotypes with distinct geographic distribution, with genotype 1 being found primarily in Europe and Asia and genotype 2 in the Americas and Asia. The two genotypes show differences in both antigenicity and severity of pathology and have >15% genome divergence between them (reviewed in [50]). Genotype 1 can be further divided into three subtypes, based on the ORF7 sequence and geographical distribution, whereby subtype 1 is pan-European whilst subtypes 2 and 3 are currently limited to Eastern Europe [51]. Here we tested all genotype 1 subtypes of PRRSV, represented by subtype 1 strain H2 (PRRSV H2) [52], subtype 2 strain DAI (PRRSV DAI) [53], and subtype 3 strain SU1-Bel (PRRSV SU1-Bel) [54], originally isolated from the UK, Lithuania, and Belarus, respectively.

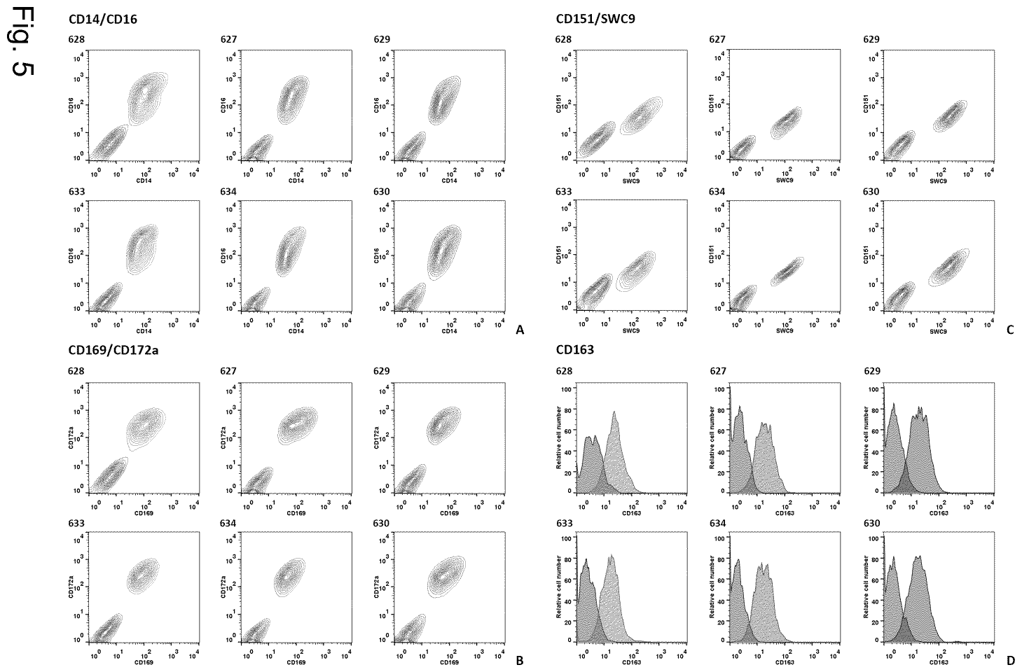

PAMs were infected at an MOI=1 in a single-round infection. 19 hours post inoculation (hpi) the cells were harvested and stained with a FITC-labelled antibody against PRRSV-N protein. Infection levels were assessed by FACS analysis. All three virus subtypes resulted in infection levels of 40-60% in wild type and heterozygous animals, with more than 98% of infected cells being classified as CD163 positive. A slightly higher, statistically significant infection was observed in heterozygous animals infected with PRRSV H2 and DAI. The reason for this is unclear, but may reflect either altered CD163 protein expression profile in heterozygous animals or other, as yet unidentified, genetic properties. By contrast, cells from both ΔSRCR5 animals (629 and 630) were found to be highly resistant to infection in this assay (FIG. 4 A-C). A second assay was performed to assess whether virus could replicate in PAMs then infect neighboring cells in a multiple-round infection time course. Cells were inoculated at MOI=0.1 and supernatant samples collected at indicated time points. Viral RNA was extracted from the supernatants and analyzed by RT-qPCR. For PRRSV H2 and SU1-Bel specific probes and primers against ORF7 were employed. To assess PRRSV DAI vRNA specific primers against ORF5 and BRYT green dye binding were used due to the limited genome information available on this strain. All wild type and heterozygous animals replicated the virus to similar levels. Virus levels started to rise by 12 hpi and increased exponentially up to 36 hpi when they plateaued. PRRSV SU1-Bel levels reached their plateau at 48 hpi. The detection limit of the RT-qPCR corresponded to a CT value of 35, which corresponded to 1E4 TCID₅₀/ml for PRRSV H2, 1E3 TCID₅₀/ml for PRRSV DAI, and 5E3 for PRRSV SU1-Bel. Viral RNA (vRNA) levels in supernatants from ΔSRCR5 PAMs in this multiple round infection did not increase above the detection limit (FIG. 4 D-F). In order to assess whether infectious virions were produced a TCID₅₀ assay was conducted on supernatant collected at 48 hpi, when all three subtypes had reached a plateau. Serial dilutions were started at a 1:10 dilution, corresponding to a detection limit of 63 TCID₅₀/ml. Virus produced from PAMs of wild type or heterozygous origin was infectious and levels measured were comparable to those calculated for the vRNA extractions. By contrast, homozygous ΔSRCR5 PAMs did not support virus production at the detection limit of this assay (FIG. 4 G-J). In summary, PAMs from ΔSRCR5 animals could not be infected by PRRSV genotype I at a high MOI nor did they replicate the virus over a 72 h time course.

Peripheral Blood Monocytes from ΔSRCR5 Pigs Differentiate into CD163-Expressing Macrophages Upon CSF1-Induction and Express Macrophage-Specific Markers

To assess the differentiation potential of monocytes into CD163-expressing macrophages we isolated peripheral blood monocytes (PBMCs) from whole blood then were differentiated them into macrophages by CSF1-induction for seven days. Expression of macrophage specific markers was assessed by immunofluorescence labelling and FACS analysis. CD14 and CD16 levels are clear indicators of the differentiation of peripheral blood monocytes with levels of both increasing significantly upon differentiation [44,46]. In addition to CD172a, CD169, and CD163, whose roles as macrophage markers are discussed above, we included a PBMC differentiation marker, SWC9, also known as CD203a, and the putative PRRSV attachment factor CD151 [55,56].

CD14/CD16 staining of the PMBC-derived macrophages (PMMs) from the ΔSRCR5, heterozygous, and wild type animals were all within the previously observed and documented levels, with no difference being observed between the genotypes (FIG. 5A). The monocyte/macrophage lineage marker CD172a was expressed at high levels in all animals and CD169 was expressed at expected levels (FIG. 5B). Expression of SWC9 highlighted the full differentiation of the PMMs. CD151 expression together with the previously shown CD169 expression demonstrated that both of these putative PRRSV attachment factors or receptors are still expressed on macrophages from ΔSRCR5 animals (FIG. 5C). As with PAMs, both the unmodified and the ΔSRCR5 CD163 proteins were detected on the surface of the PMMs (FIG. 5D). The medians of CD163 fluorescence intensity of pigs 628, 633, 627, 634, 629, 630 were 23.3, 16.7, 18.3, 16.5, 18.8, and 17.2, respectively, with the isotype control medians ranging from 1.88-3.79. This indicates slightly lower expression levels of CD163 on PMMs compared to PAMs. Overall, PBMCs isolated from all animals, independent of their genotype were shown to be fully differentiated into PMMs upon rhCSF1 induction. They all expressed macrophage-specific surface markers, including CD169, CD151, and CD163, which have putative functions in PRRSV entry.

ΔSRCR5 Peripheral Blood Monocyte-Derived Macrophages Still Function as CD163-Dependent Hemoglobin-Haptoglobin Scavengers.

In addition to its contribution to PRRSV susceptibility, CD163 has been described to have a variety of important biological functions. CD163 is an erythroblast binding factor, enhancing the survival, proliferation and differentiation of immature erythroblasts, through association with SRCR domain 2 and CD163 expressing macrophages also clear senescent and malformed erythroblasts. SRCR domain 3 plays a crucial role as a haemoglobin (Hb)-haptoglobin (Hp) scavenger receptor. Free Hb is oxidative and toxic; once complexed with Hp is cleared through binding to SRCR3 on the surface of macrophages and subsequent endocytosis. This prevents oxidative damage, maintains homeostasis, and aids the recycling of iron. CD163 expressing macrophages were also found to be involved in the clearance of a cytokine named TNF-like weak inducer of apoptosis (TWEAK), with all SRCRs apart from SRCR5 being involved in this process [57]. Soluble CD163 can be found at a high concentration in blood plasma but its function in this niche is still unknown (reviewed in [34,58]). Maintaining these biological functions is likely to be important to the production of healthy, genetically edited animals. Interestingly, none of the biological functions assigned to CD163 have yet been linked to SRCR5. In order to confirm whether ΔSRCR5 macrophages were still able to take up Hb-Hp complexes we performed a variety of in vitro experiments. Hb-Hp complex uptake in PMMs in vitro has been investigated extensively in the past, with PMMs able to take up both Hb and Hb-Hp complexes in a CD163-dependent manner and the inducible form of heme oxygenase, heme oxygenase 1 (HO-1), being upregulated upon Hb-Hp uptake [59,60].

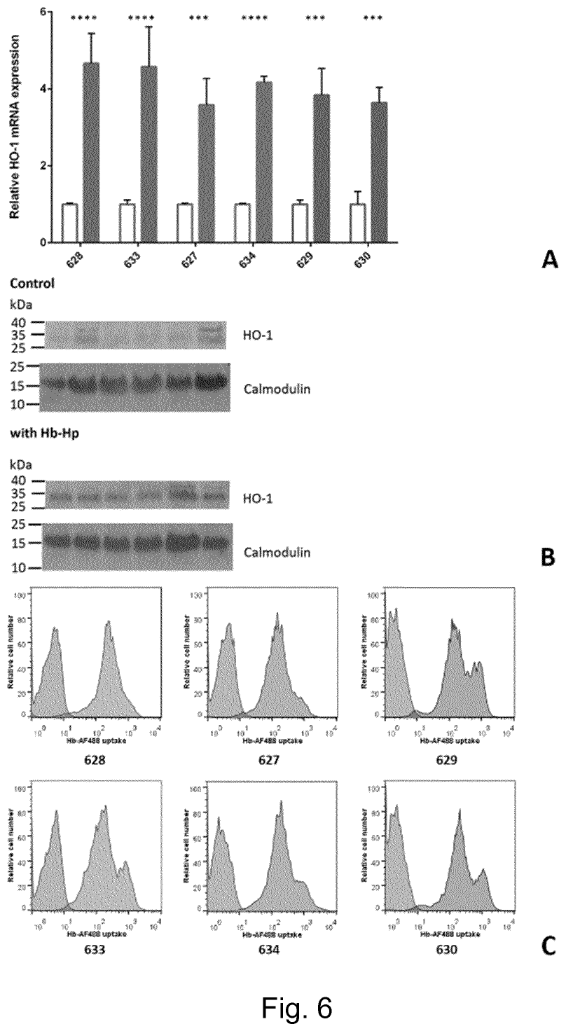

PBMCs were differentiated into PMMs by CSF1-induction for seven days, following which PMMs were incubated in the presence of Hb-Hp for 24 h to stimulate HO-1 upregulation. The HO-1 mRNA upregulation, assessed by RT-qPCR, increased 2- to 6-fold in the PMMs from all animals (FIG. 6A) with no significant difference between the different genotypes. To assess HO-1 levels by western blotting PMMs were incubated in the presence of Hb-Hp for 24 h, lysed using reducing Laemmli sample buffer, and proteins separated by SDS-PAGE. The levels of HO-1 were assessed using a monoclonal antibody against the protein, with a monoclonal antibody against calmodulin as a loading control. HO-1 protein expression was found to be upregulated in all animals, independent of CD163 genotype (FIG. 6B). To assess the uptake of Hb-Hp directly Hb was labelled with Alexa Fluor 488 (AF488). PMMs were incubated with Hb_{AF488}-Hp for 30 min and followed by FACS analysis. Independent of the CD163 genotype, Hb_{AF488}-Hp was taken up efficiently by the PMMs with medians of green fluorescence being 329, 305, 329, 366, 340, and 405 for animals 628, 633, 627, 634, 629, and 630, respectively, whilst the background mock-treated cell medians ranged from 2.41-4.74 (FIG. 6C). The uptake of Hb_{AF488}-Hp into the PMMs was confirmed by confocal microscopy. In a further experiment PMMs were incubated with Hb_{AF488}-Hp for 30 min, followed by fixation and staining for CD163. The Hb_{AF488}-Hp was found in distinct spots, presumably endosomes, with no obvious co-localization with CD163. This lack of colocalization is not surprising as the majority of Hb_{AF488}-Hp complexes observed were likely already located in late endosomes and lysosomes. Overall, this data demonstrates that macrophages from ΔSRCR5 animals retain the ability to perform their role as hemoglobin-haptoglobin scavengers.

Peripheral Blood Monocyte-Derived Macrophages from ΔSRCR5 Animals are not Susceptible to Infection with PRRSV Genotype 1

To explore the possibility that PMMs could be a suitable alternative to monitor PRRSV infection and investigate whether ΔSRCR5 PMMs, like PAMs, are resistant to PRRSV infection we tested infectivity with all three genotype 1 subtypes of PRRSV, represented by the strains described above.

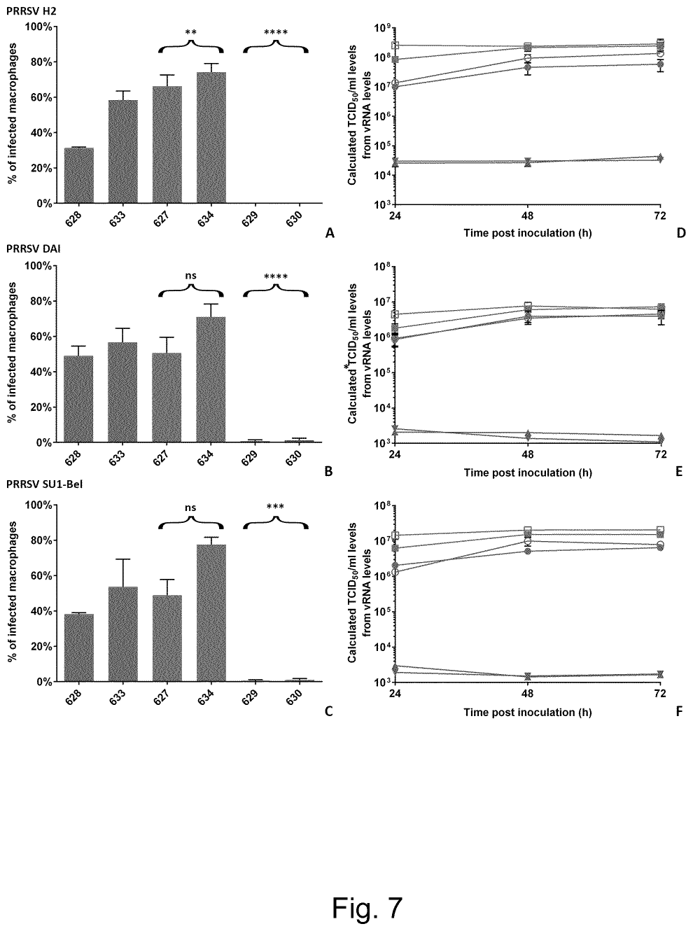

PMMs were infected at an MOI=1 in a single-round infection. 19 hpi cells were harvested and stained with a FITC-labelled antibody against PRRSV-N protein, with infection levels assessed by FACS. All three subtypes showed infection levels of 35-80% in wild type and heterozygous animals. As observed in PAMs, a slightly higher, statistically significant infection was observed in heterozygous animals infected with PRRSV H2, whilst no significant infection was observed in the cells from ΔSRCR5 animals (FIG. 7 A-C). To assess whether virus would be replicated on PMMs from the different CD163 genotypes a multiple-round infection was conducted. Cells were inoculated at MOI=0.1 and samples were collected at time points throughout the plateau stage of infection (24, 48, and 72 hpi as identified during the PAM infection time courses). Viral RNA was extracted from the supernatants and analyzed by RT-qPCR. All wild type and heterozygous animals replicated the virus at similar levels. Interestingly, PMMs replicated all viruses to higher levels than PAMs, suggesting that PMMs are not only suitable but may in fact be a superior model for in vitro infection studies with PRRSV. The detection limits of the RT-qPCR were identical to those described above. No replication of PRRSV was observed in ΔSRCR5 animals (FIG. 7 D-F).

The Arrest in Infection of ΔSRCR5 Pulmonary Alveolar Macrophages (PAMs) Occurs Prior to the Formation of the Replication/Transcription Complex.

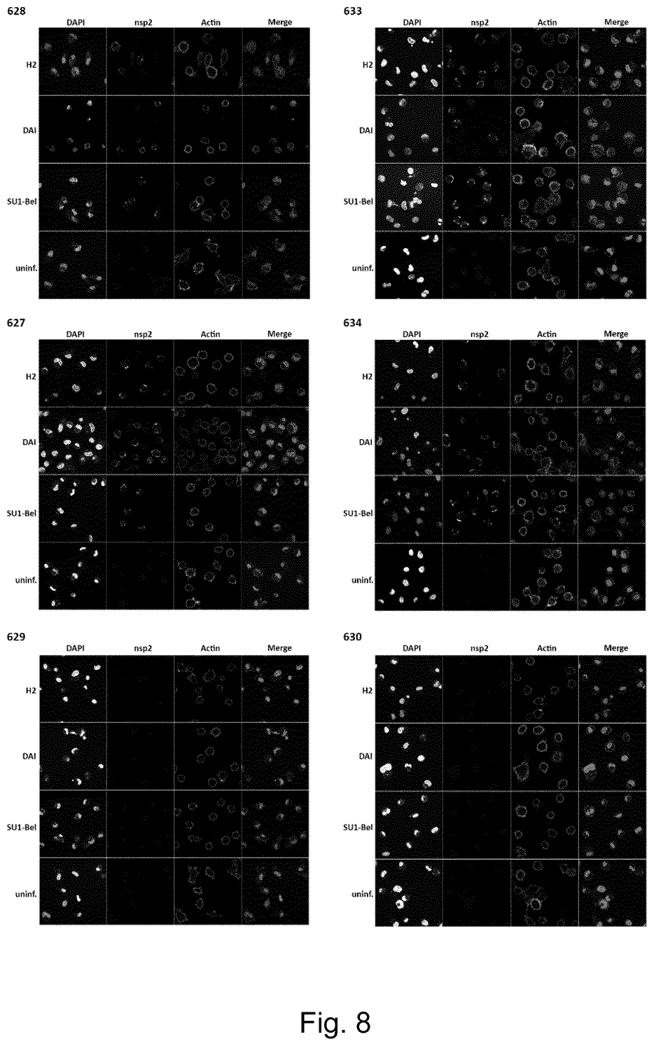

In the porcine kidney cell line PK-15, lacking CD163 expression, transfected with the PRRSV attachment factor CD169 the virus was found to be internalized but not to undergo uncoating [36]. This indicates that CD163, in a close interplay with attachment/internalization factors, plays a major role in the entry process of PRRSV. To assess whether the infection process in ΔSRCR5 macrophages is arrested prior to replication we inoculated PAM cells with all three PRRSV genotype 1 subtypes, represented by the strains described above, at MOI=2. The inoculum was removed 3 hpi and infection allowed to continue up to 22 hpi. Cells were fixed and stained for the replication-transcription complexes (RTC) formed by PRRSV upon replication initiation. PRRSV nsp2 protein, involved in the formation of double membrane vesicles (reviewed in [61]) was chosen as a representative marker for the RTC. The cells were permeabilized and stained for the presence of PRRSV nsp2. We found that macrophages from both the wild type and the heterozygous animals infected with PRRSV formed RTCs, independent of the subtype. However, in the macrophages from ΔSRCR5 animals no RTC formation was observed. This underlines the involvement of CD163 in the entry and uncoating process of PRRSV infection. It also supports the deletion of SRCR5 as an effective method to abrogate PRRSV infection before the virus or viral proteins are amplified (FIG. 8).

### DISCUSSION

The results of this study show that live pigs carrying a CD163 SRCR5 deletion are healthy and maintain the main biological functions of the protein, whilst the deletion renders target cells of PRRSV resistant to infection with the virus. By using two sgRNAs flanking exon 7 of CD163 in CRISPR/Cas9 editing in zygotes we achieved excision of said exon from the genome of pigs yielding a CD163 ΔSRCR5 genotype. The expression of the truncated gene was confirmed by PCR of cDNA, RT-qPCR and western blotting against CD163. Macrophages isolated from the lungs of wild type CD163, heterozygous and ΔSRCR5 animals showed full differentiation and expression of macrophage surface markers characteristic of macrophages isolated from the pulmonary alveolar areas. PAMs are the primary target cells of PRRSV infection. Assessing infection of PAMs from the different genotype animals in both high dose, single-round infections and low dose, multiple-round infections showed PAMs from ΔSRCR5 pigs to be resistant to infection in vitro. The differentiation ability of cells of the monocytes/macrophages lineage from genetically edited CD163 animals was further confirmed by isolation and differentiation of PBMCs. PMMs from ΔSRCR5 pigs were also shown to be resistant to PRRSV infection. PMMs have a crucial biological role, serving as scavengers for Hb-Hp complexes in the blood. Using uptake experiments of fluorescently labelled Hb-Hp complexes as well as gene upregulation assays to monitor the increase of HO-1 upon Hb-Hp stimulation we confirmed that this important biological function is maintained in macrophages isolated from ΔSRCR5 animals.

Using CRISPR/Cas9 editing in zygotes generated live pigs with exon 7 CD163 deletions. Editing efficiency was highly variable, dependent on surgery days, in both in vitro cultivated blastocysts as well as born animals, whereby it needs to be considered that overall numbers are low. The reagents used on the various surgery days were the same and both insemination and surgery times were kept consistent. However, there are many elements in the genome editing process that rely on highly skilled personnel and technical reproducibility. Recent developments in nucleic acid delivery methods for genome editing in zygotes may offer possible solutions to standardize the genome editing process. Various groups recently reported successful genome editing by in vitro electroporation of CRISPR/Cas9 regents into zygotes isolated from mice and rats without removing the zona pellucida [62-64]. Using electroporation to deliver genome editing reagents in vivo Takahasi et al. showed high success with this method in mouse embryos after 1.6 days of gestation [65]. Use of in vitro electroporation could standardize the injection process and reduce the requirement for highly trained personnel. As an alternative, in vivo electroporation would remove both the requirement for donor animals and the long handling process of zygotes prior to re-implantation, however this procedure has currently only been developed for mice (reviewed in [66]). Three out of four of the founder animals were found to be edited in a mosaic pattern. In animal 310 the mosaicism seems to result from a delayed activity of the CRISPR/Cas9 complex, resulting in an edit of one allele in a single cells at the 4- or 8-cell stage. In animals 345 and 347 an initial editing event appears to occur in one allele at the 1-cell stage and a second editing event, modifying the second allele in one of the cells at the 2-cell stage, resulting in homozygous/heterozygous mosaic animals. Mosaicism has been observed in various studies employing injection of genome editors into porcine zygotes [67-69]. Asymmetric spreading of introduced mRNA seems unlikely following results of Sato et al., who performed in vitro EGFP mRNA injections using parthenogenetically activated porcine oocytes, whereby a relatively homogenous fluorescence pattern could be observed [69]. Rather, mosaicism seems to result from Cas9 protein/sgRNA complexes remaining active throughout several cell divisions or delayed mRNA expression possibly triggered by cell division. The former theory is supported by the genotype of 345 and 347, which very likely have developed from an initial editing step in one allele at the one cell stage and editing of the second allele in one of the 2-cell or 4-cell stage cells. To generate more biallelic animals by direct injection of zygotes, a more active reagent set may be beneficial. Recent studies indicate that injection of Cas9/sgRNA ribonucleoproteins (RNPs) is more efficient than mRNA injection. Also, RNP injection can be combined with in vitro electroporation [70].

The mating of the F0 generation animals 310 and 345 resulted in wild type, heterozygous and biallelic edited animals. This showed that despite mosaicism both animals are germline heterozygous. None of the offspring showed any adverse effect from the genome editing under standard husbandry conditions. Interestingly, one of the animals, 630, displayed a putative gene conversion event. Based on the mechanism of interallelic gene conversion we assume that a homologous recombination occurred in this animal between one allele showing the edited genotype of 345 and the other allele the edited genotype of 310. The gene conversion appears to have occurred at the zygote stage, rendering 630 homozygous for the genotype of 310 (reviewed in [71]).

PRRSV shows a very narrow host cell tropism, only infecting specific porcine macrophage subsets. Isolating these cells from our genetically edited animals and their wild type siblings we showed that removal of the CD163 SRCR5 domain results in complete resistance of the macrophages towards PRRSV infection. We further demonstrated that ΔSRCR5 animals are resistant to infection with all European subtypes of genotype 1. This shows that a targeted removal of SRCR5 is sufficient to achieve complete resistance to PRRSV infection in vitro. PRRSV attachment factors CD151 and CD169 are still expressed on ΔSRCR5 macrophages underlining that these proteins are not sufficient for PRRSV infection. PRRSV infection on macrophages from the ΔSRCR5 animals was halted before the formation of replication transcription complexes proving CD163 to be involved in the entry or uncoating stage of the PRRSV replication cycle. The ΔSRCR5 macrophages will provide a new tool to study this process in detail in a true-to-life system.

As there could be a genetic variation of CD163 within the Suidae superfamily we performed an in vitro control experiment to assess the susceptibility of warthog (Phacocherus africanus) PMMs to PRRSV infection. Interestingly, warthog PMMs were found to be as susceptible to infection with all PRRSV genotype 1 subtypes as the pig PMMs. They all replicated the virus at a similar rate and to comparable titers (data not shown). This indicates that genetic variation of CD163 within the Suidae superfamily is probably very limited and PRRSV infection may be widespread. This also shows that the virus poses a threat to African pig breeding countries. The ΔSRCR5 animals have several advantages over previously described genome edited animals resistant to PRRSV infection. Whitworth et al. generated animals with a premature stop codon in exon 3 of the CD163 gene, resulting in an ablation of CD163 expression [37]. In contrast to this we have demonstrated that specific application of genome editing tools in vivo can be used to efficiently generate animals with precise deletion of exon 7 of CD163, and that these animals retain expression of the remainder of the CD163 protein on the surface of specific differentiated macrophages in a native conformation. We further showed that the macrophages from these ΔSRCR5 animals retain full differentiation potential, both in PAMs as well as PBMCs stimulated to differentiate by CSF-1 addition, and that macrophages from edited animals retain the ability to perform crucial biological functions associated with CD163 expression, such as hemoglobin/haptoglobin uptake. Overall, this study demonstrates that it is possible to utilize a targeted genome editing approach to render swine resistant to PRRSV infection, whilst retaining biological function of the targeted gene. Introduction of CD163 SRCR5 deletion animals in pig breeding could significantly reduce the economic losses associated with PRRSV infection.

Inactivation of Splice Acceptor Site in Intron 6

An alternative strategy to delete the SRCR5 domain of CD163 is to inactivate the splice acceptor site located at the 5′ end of exon 7 in the CD163 gene.

Inactivation of the splice acceptor site in exon 7 can be achieved in a number of ways, and two suitable strategies are discussed briefly below, one involving creating a double stranded cut followed by non-homologous end joining (NHEJ), and the other using homology directed repair (HDR). The first option suitably uses a single guide RNA and NHEJ by the target cell. Using the second approach, HDR, a template is provided which is used by the cell's double strand break repair machinery to introduce a sequence modification. Thereby some nucleotides will be replaced in order to destroy the splice acceptor site in a targeted manner, whilst introducing a restriction site (in the example NcoI) which allows for convenient confirmation that the HDR event has taken place.

Suitable methodologies for achieving editing events in pig embryos and generation of animals from edited embryos are discussed above, and are also extensively discussed in the literature, and thus for conciseness they will not be repeated here.

In the case of CRISPR/Cas9 mediated gene editing, suitable guide RNA sequences to target the splice acceptor site are as follows:

sgRNA 1:
(SEQ ID NO: 12)
AACCAGCCTGGGTTTCCTGT
sgRNA 2:
(SEQ ID NO: 13)
CAACCAGCCTGGGTTTCCTG

These two guide sequences result in the induction of double stranded cut sites at the following sequences at the 5′ end of exon 7 by Cas9:

(SEQ ID NO: 14)
ACA|GGAAACCCAGGCTGGTT - using sgRNA 1
(SEQ ID NO: 15)
CAG|GAAACCCAGGCTGGTTG - using sgRNA 2

Approach 1—NHEJ

An RNP complex of sgRNA1 or 2 with Cas9 binds to the target site in the CD163 gene and causes a double-strand break. Where the break occurs NHEJ events arise, commonly resulting in and insertion of deletion event. It is highly likely that either insertion or deletion events will result in the inactivation of the intron 6 splice acceptor site. It is thereafter simply a matter of identifying embryos having the requisite disabling of the splice acceptor site.

Approach 2—HDR

Again, an RNP complex of sgRNA1 or 2 with Cas9 binds to the target site in the CD163 gene and causes a double-strand break. In this case, however, an HDR template is provided, for example a single or double stranded DNA molecule, which comprises a sequence which results in a change of the sequence in the CD163 gene from:

(SEQ ID NO: 3)
AATGCTATTTTTCAGCCCACAGGAAACCCAGG
to:
(SEQ ID NO: 4)
AATGCTATTTTTCgGCCatggGGAAACCCAGG

A suitable HDR template has the following sequence: GAAGGAAAATATTGGAATCATATTCTCCCTCACCGAAATGCTATTTTTCgGCCatggGGAAAC CCAGGCTGGTTGGAGGGGACATTCCCTGCTCTGGTC (SEQ ID NO:16—lower case letters show the changes made compared to the unaltered sequence).

The converted sequence in the context of CD163 results in inactivation of the splice acceptor site and the introduction of the NcoI restriction site. The presence of the NcoI site facilitates identification of embryos/animals in which the desired HDR edit has been achieved.

Further Experimental Work

Genome Editing in Zygotes for ΔSRCR5 CD163 Pigs and Breeding for a Genotypically Uniform F2 Generation

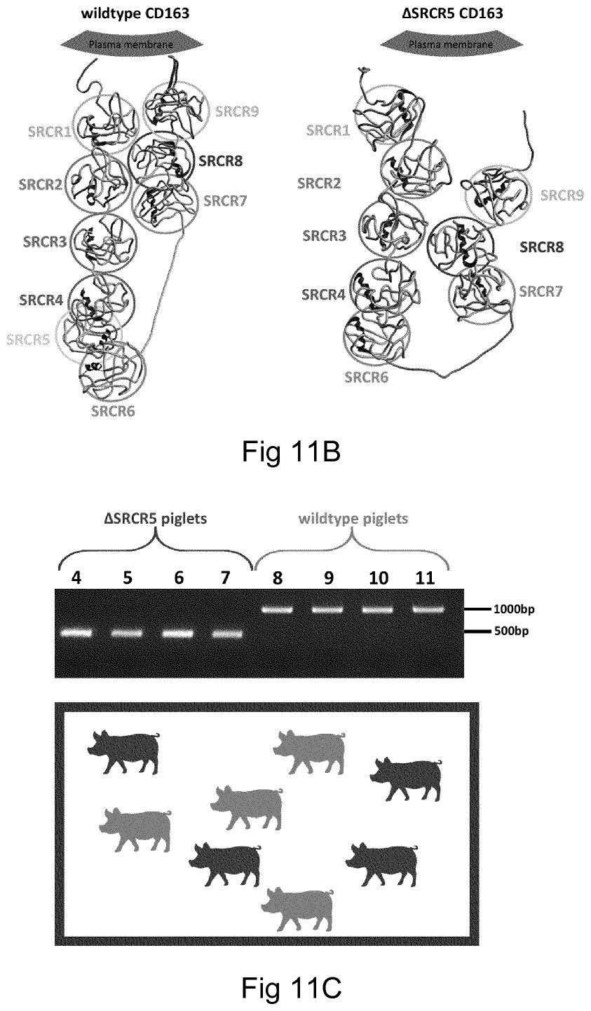

Founder generation F0 animals carrying a deletion of exon 7 in the CD163 gene, which encodes the scavenger receptor cysteine-rich domain 5 (SRCR5) of the protein, were generated by CRISPR/Cas9 gene editing as described above (see also 75). Therefore, zygotes were microinjected with two guide RNAs, sgSL26 and sgSL28, in combination with Cas9 mRNA to achieve CRISPR/Cas9-mediated double-strand breaks (DSBs) flanking exon 7. Subsequent DSB repair lead to a deletion of exon 7 (FIG. 11A). Breeding of heterozygous founder animals and with wildtype pigs yielded a first generation of heterozygous and biallelic edited animals (F1 generation). At this stage we selected heterozygous F1 animals displaying a “clean” ligation, i.e. without any insertions or deletions at this site, at the cutting sites of sgSL26 and sgSL28 for further breeding. Half-sibling heterozygous animals and wildtype animals were bred to yield a lineage of homozygous ΔSRCR5 animals carrying the “clean-cut” genotype (FIG. 11A) and wildtype sibling and semi-sibling animals with a similar genetic background.

As previously described, ΔSRCR5 animals express the ΔSRCR5 CD163 mRNA and protein at equivalent levels to wildtype siblings. Furthermore, native-structure ΔSRCR5 CD163 is recognized on the surface of pulmonary alveolar macrophages (PAMs) by a respective antibody. We have further analyzed whether template-based protein structure prediction using RaptorX confirms these findings towards proper folding of the subdomains and the complete ΔSRCR5 CD163 protein (39). As seen in FIG. 1B, all subdomains in both the full-length and ΔSRCR5 CD163 are predicted to adopt the globular structure and a pearl-on-a-string configuration. This supports our findings towards proper folding and expression of the ΔSRCR5 protein.

Previously, we have shown that PAMs and in vitro differentiated peripheral blood monocytes are resistant to infection with both, porcine reproductive and respiratory syndrome virus 1 (PRRSV-1) and PRRSV-2. Now, we aimed to confirm the in vitro results by assessing resistance towards PRRSV-1 infection in vivo. Therefore, we selected four homozygous ΔSRCR5 F2 animals and four wildtype siblings and semi-siblings. The animals were co-housed from weaning. At 6 weeks of age they were transferred to the specific pathogen-free (SPF) unit and co-housed for the duration of the challenge (FIG. 11C).

ΔSRCR5 Pigs Show Normal Whole Blood Counts and Soluble CD163 Serum Levels

Prior to being moved to the SPF unit blood samples were taken from all eight pigs and analyzed by a full blood count conducted by the diagnostics laboratory at the Royal (Dick) School of Veterinary Studies, University of Edinburgh. The blood counts of all animals were within reference values indicating good general health and the absence of infection or inflammation. Furthermore, the hemoglobin levels of all animals were within reference values, indicating normal function of the hemoglobin/haptoglobin scavenging activity of CD163 (Table 2).

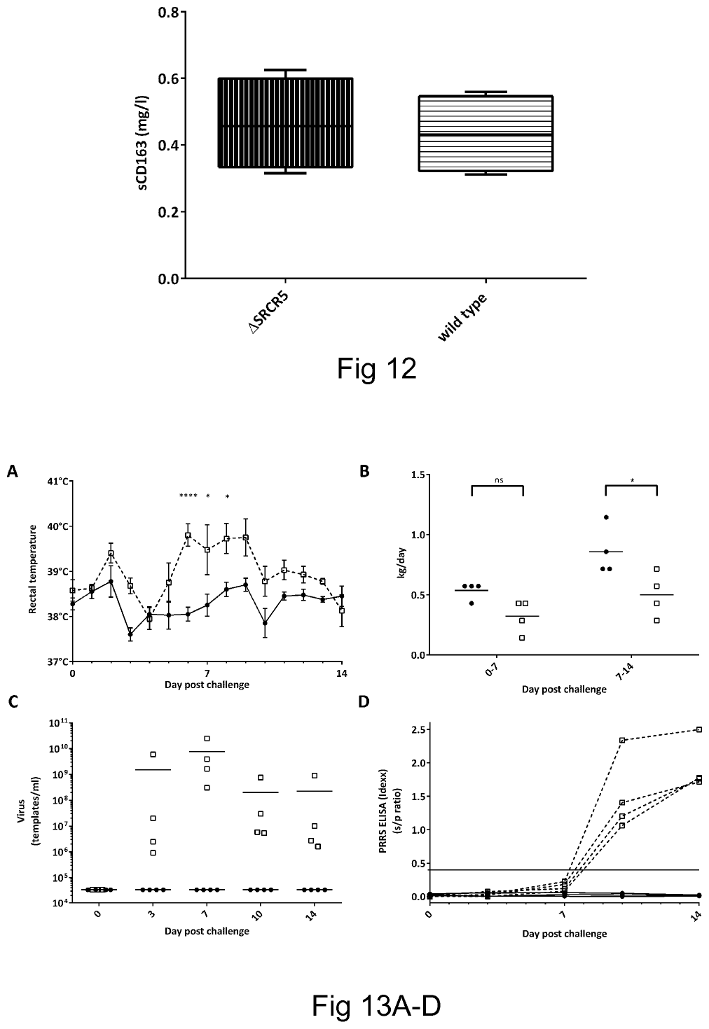

Serum was collected from all animals prior to movement to the SPF unit and on day 0 prior to challenge with PRRSV-1. The soluble CD163 (sCD163) serum levels were assessed using a commercially available enzyme-linked immunosorbent assay (ELISA) recognizing soluble porcine CD163. Serum CD163 levels were found to be 463.5±68.99 ng/ml in ΔSRCR5 pigs and 433.2±69.57 ng/ml in wildtype pigs (FIG. 12). These levels are comparable to sCD163 levels in humans (for example (76)) and not significantly different from each other.

TABLE 2
Whole blood count results of ΔSRCR5 & wildtype piglets at 5.5 weeks of age. 4-7
ΔSRCR5, 8-11 wildtype pigs.

| | | | | | | | | | | Ref |
|---|---|---|---|---|---|---|---|---|---|---|
| Indicator | 4 | 5 | 6 | 7 | 8 | 9 | 10 | 11 | Unit | Values |
| WBC | 22.5 | 24 | 14 | 15.1 | 12.4 | 19.6 | 26.1 | 14.4 | ×10⁹/l | 11-22 |
| Neutrophils | 5.85 | 4.8 | 4.62 | 5.889 | 4.34 | 7.252 | 7.83 | 4.32 | ×10⁹/l | 2-15 |
| (segmented) | | | | | | | | | | |
| Neutrophils | 26 | 20 | 33 | 39 | 35 | 37 | 30 | 30 | % | 20-70 |
| (segmented) | | | | | | | | | | |
| Neutrophils | 0 | 0 | 0 | 0 | 0 | 0 | 0 | 0 | ×10⁹/l | 0-0.8 |
| (non- | | | | | | | | | | |
| segmented) | | | | | | | | | | |
| Neutrophils | 0 | 0 | 0 | 0 | 0 | 0 | 0 | 0 | % | 0-4 |
| (non- | | | | | | | | | | |
| segmented) | | | | | | | | | | |
| Lymphocytes | 15.3 | 18.72 | 8.82 | 8.305 | 7.564 | 11.76 | 16.182 | 9.36 | ×10⁹/l | 3.8-16.5 |
| Lymphocytes | 68 | 78 | 63 | 55 | 61 | 60 | 62 | 65 | % | 35-75 |
| Monocytes | 0.675 | 0.48 | 0.42 | 0.755 | 0.496 | 0.588 | 1.044 | 0.576 | ×10⁹/l | 0-1 |
| Monocytes | 3 | 2 | 3 | 5 | 4 | 3 | 4 | 4 | % | 0-10 |
| Eosinophils | 0.675 | 0 | 0 | 0.151 | 0 | 0 | 1.044 | 0.144 | ×10⁹/l | 0-1.5 |
| Eosinophils | 3 | 0 | 0 | 1 | 0 | 0 | 4 | 1 | % | 0-15 |
| Basophils | 0 | 0 | 0.14 | 0 | 0 | 0 | 0 | 0 | ×10⁹/l | 0-0.5 |
| Basophils | 0 | 0 | 1 | 0 | 0 | 0 | 0 | 0 | % | 0-3 |
| RBC | 6.03 | 6.64 | 6.99 | 6.58 | 6.3 | 6.53 | 7.52 | 6.97 | ×10¹²/l | 5-9 |
| PCV/ | 0.384 | 0.391 | 0.383 | 0.388 | 0.382 | 0.39 | 0.429 | 0.421 | | 0.36-0.43 |
| Hematocrit | | | | | | | | | | |
| Hb | 11.5 | 11.9 | 10.9 | 11.8 | 11.6 | 12 | 13.8 | 12.3 | g/dl | 10-16 |
| MCV | 63.7 | 58.9 | 54.8 | 58.9 | 60.7 | 59.8 | 57.1 | 60.5 | fL | 50-62 |
| MCHC | 29.9 | 30.4 | 28.3 | 30.5 | 30.3 | 30.9 | 32.1 | 29.1 | g/dl | 30-36 |
| Platelets | 219 | 230 | 605 | 397 | 483 | 519 | 219 | 606 | | 120-720 |
| RDW | 20.9 | 23.1 | 28.9 | 20.6 | 21 | 18 | 17 | 22.6 | | |

ΔSRCR5 Pigs Show No Signs of PRRSV-1 Infection

At 7-8 weeks of age the pigs were inoculated intranasally with the PRRSV-1, subtype 2 strain BOR-57 (77). Generally, infections with PRRSV-1, subtype2 strains are associated with mild respiratory symptoms, elevated body temperature, extensive lung pathology and high viremia. The challenge was conducted for a period of 14 days following inoculation at day 0 and day 1 with 5E6 TCID₅₀ of the virus each. Rectal temperature, respiratory and other potential symptoms, and demeanor were recorded each day and serum samples were collected on day 0 (prior to challenge), 3, 7, 10, and 14 (prior to euthanasia). Weights were recorded on day 0, 7, and 14 (prior to euthanasia). People conducting the challenge and analyzing the pathology were blind to the genotype of the animals.

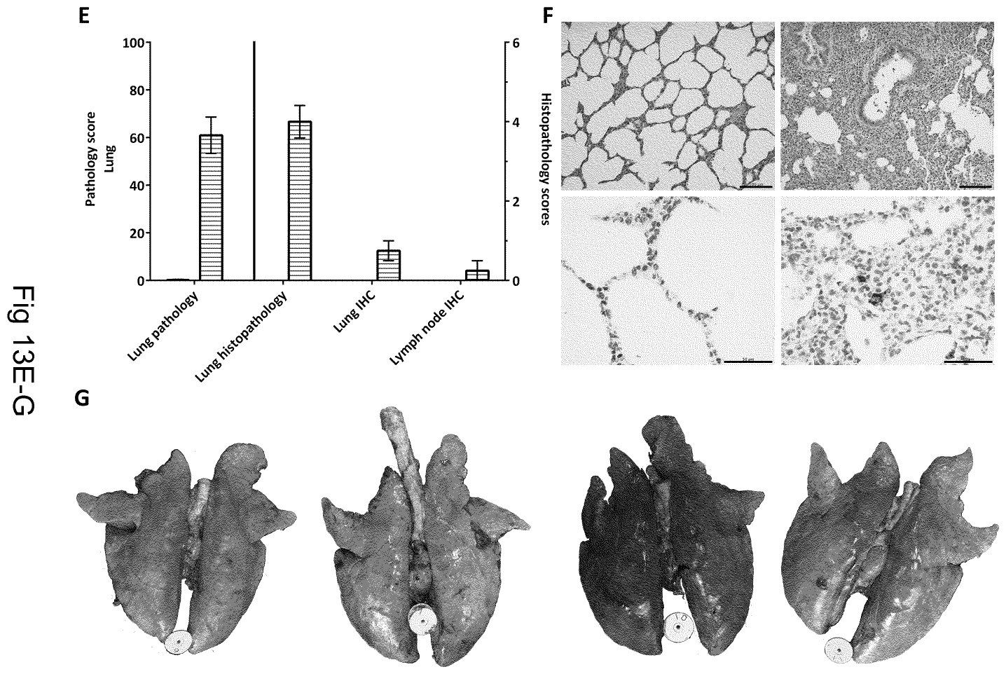

The rectal temperature showed significant elevations on days 6-9 of the challenge in the wildtype animals, whereas no body temperature increase was observed in the ΔSRCR5 animals (FIG. 13A). The average daily weight gain of the ΔSRCR5 pigs was higher compared to their wildtype counterparts over the entire challenge period and significantly so over days 7-14 (FIG. 13B). Only one wildtype pig showed changed demeanor on days 7 to 8, other than that, no respiratory symptoms or other abnormalities in behavior were observed. Viral RNA was isolated from serum and virus levels quantified using a DNA fragment template standard and viral RNA extracted from known infectivity virus stocks. Whereas the wildtype pigs showed a high viremia no viral RNA was detected in the serum of ΔSRCR5 pigs (FIG. 13C). The presence of antibodies against PRRSV was assess using a commercial ELISA able to detect antibodies against all PRRSV-1 subtypes and PRRSV-2. PRRSV antibodies were detected in wildtype pigs from day 7 and present at significant levels on days 10 and 14 (FIG. 13D). During necropsy lungs were assessed initially and details photographs taken from the dorsal and ventral side. Lungs were scored towards the presence of lung lesions. Therefore, an established scoring system, based on the approximate contribution of each lung section to the complete lung volume was employed (78). The average lung lesion score for the wildtype animals was 61 compared to 0.25 in ΔSRCR5 pigs (FIGS. 13 E & G). Samples of the lungs were fixed in formalin, embedded in paraffin, cut into sections, and stained for further analysis. To assess general lung histology samples were stained with hematoxilyn and eosin. Sections from each pig were assessed towards the presence of interstitial pneumonia on a scale of 0-6 (0, normal; 1, mild multifocal; 2, mild diffuse; 3, moderate multifocal; 4, moderate diffuse; 5, severe multifocal; 6, severe diffuse). The lung histology score of the wildtype animals averaged 4 compared to 0 in ΔSRCR5 pig lungs (FIGS. 13 E & F, top). The presence of PRRSV antigens was assessed by immunohistochemistry on lung sections and lymph node sections using a mixture of two different antibodies against the PRRSV-N protein as described before (79). No PRRSV antigens were detected in sections from ΔSRCR5 but PRRSV antigen was detected in 3 out of 4 animals' lung sections and 1 out of 4 lymph node sections of wildtype animals (FIGS. 13 E & F, bottom).

Overall, no signs of infection were detected in ΔSRCR5 animals despite the high inoculation volume and exposure to infected and shedding wildtype animals showing that ΔSRCR5 animals are resistant to PRRSV-1 infection, confirming the results found in vitro with both PRRSV-1 and PRRSV-2.

ΔSRCR5 Pigs Show No Cytokine Response to PRRSV-1 Infection and Generally Normal Cytokine Levels

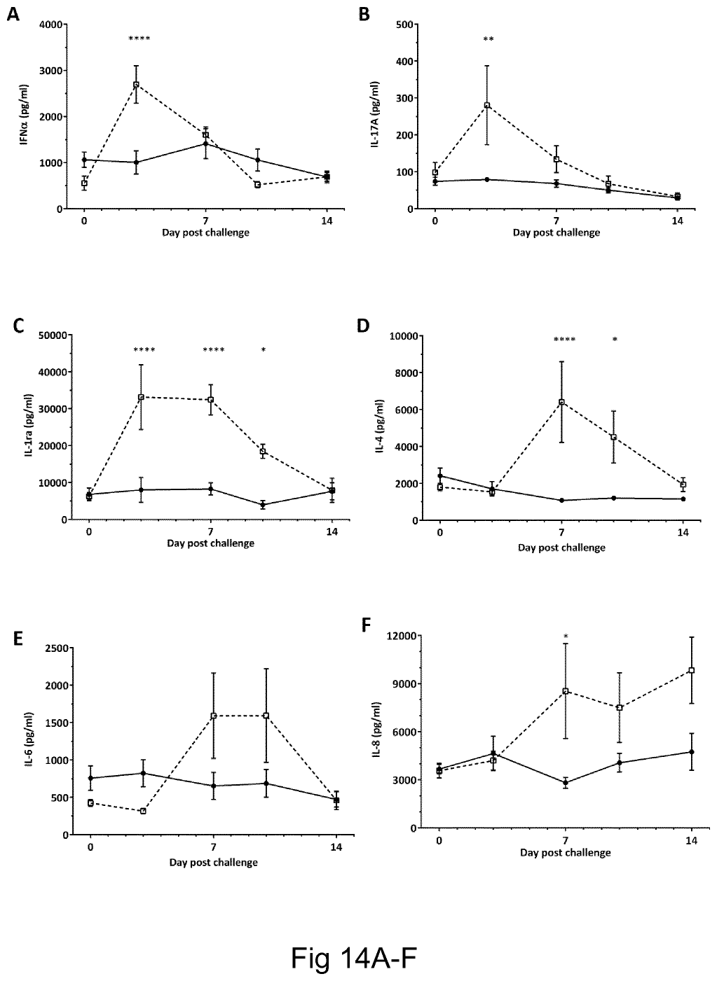

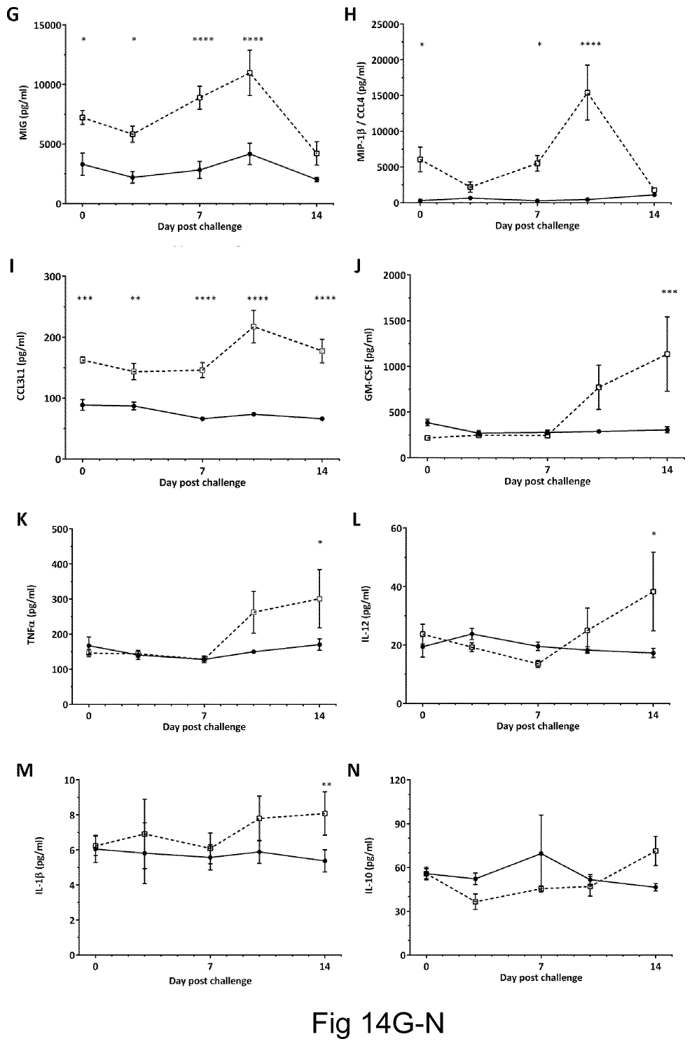

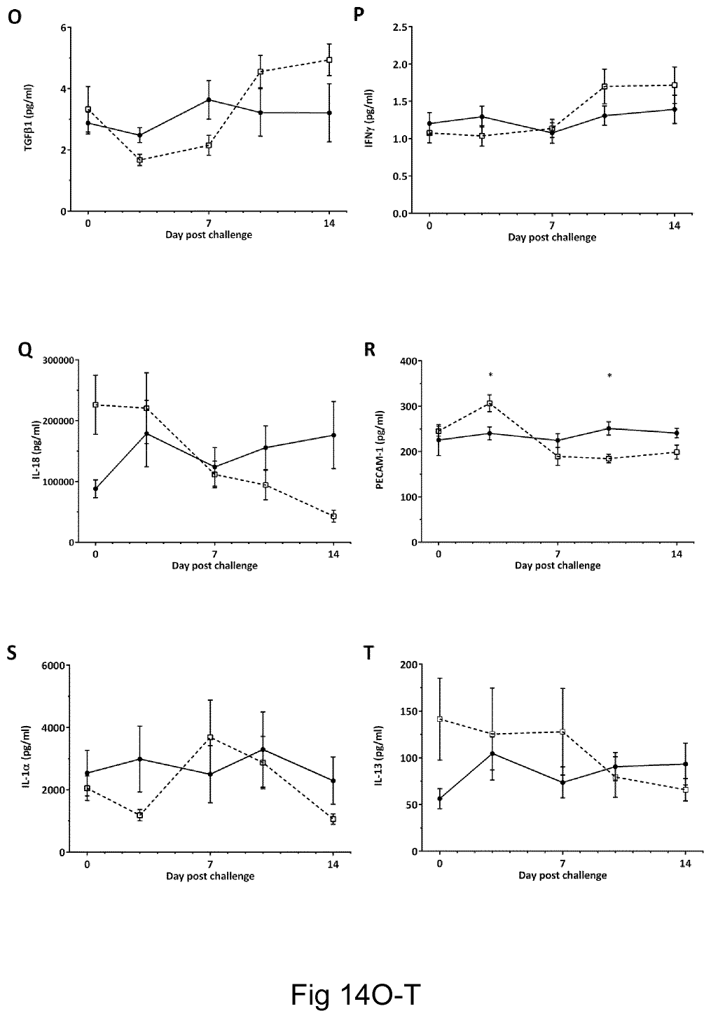

To assess the inflammation and infection response following PRRSV-1 infection a panel of 20 cytokines were analyzed towards their level in the serum of the pigs. Therefore, we used commercial quantitative antibody arrays and serum samples collected on day 0 (prior to challenge), 3, 7, 10, and 14 of the challenge. Overall, cytokine levels on day 0, considered a baseline, were similar between ΔSRCR5 and wildtype pigs. The monokine induced by gamma interferon (MIG, also known as CXCL9) was found to show consistently higher levels in wildtype pigs until day 14, when no significant difference was detected anymore. MIG is a T-cell chemoattractant to inflammation sites and involved in repair of tissue damage. In wildtype animals MIG was strongly upregulated on days 7 and 10 of the challenge (80) (FIG. 14H). Also, the chemokine ligand 3-like 1 (CCL3L1) was found to be higher in wildtype compared to ΔSRCR5 animals (FIG. 14J). CCL3L1 is involved in inflammation response and downregulated by IL-10. In wildtype animals CCL3L1 was elevated in the serum on days 10 and 14, whereas no significant IL-10 elevation was found to occur over the period of the challenge (FIG. 14O). (80,81)

Otherwise we could see a sequence of cytokine response, with early increase of interferon α (IFNα) and interleukin-17A (IL-17A), and the interleukin 1 receptor antagonist (IL-1ra) (FIGS. 14A, B, and C). This was followed by an increase in interleukins 4, 6, and 8 (IL-4, IL-6, and IL-8, respectively) at the high point of viremia, from 7 days post inoculation (dpi) (FIGS. 14 D, E, and F). Increased levels of MIG, and the macrophage inflammatory protein 1β (MIP-1β, also known as CCL4) were only observed transiently at 10 dpi (FIGS. 14 G and H). Only in the last period of the challenge, with moderate viremia levels, were elevations of CCL3L1, granulocyte macrophage colony stimulating factor (GM-CSF), interleukin 12 and 1β (IL-12 and IL-1β) detected (FIGS. 14 I, J, K, L, and M). For all these cytokines found to elevate in wildtype animals, no cytokine response was observed in ΔSRCR5 pigs. IL-10, transforming growth factor β1 (TGFβ1), and interferon γ (IFNγ) showed no significant difference in the wildtype compared to the levels in the ΔSRCR5 pigs at each time point but were found to change significantly over time in the wildtype animals (calculated using a two-way ANOVA) (FIG. 14 N, O, P). Interleukin 18 (IL-18) levels decreased significantly over time in wildtype animals but were not significantly different from those of ΔSRCR5 pigs at each time point (FIG. 14 Q). Platelet endothelial cell adhesion molecule (PECAM1) was significantly elevated on day 3 of the challenge and decreased on day 10 compared to levels of ΔSRCR5 pigs (FIG. 14 R). No significant differences in levels of interleukin 1α (IL-1α) and interleukin 13 (IL-13) were found between ΔSRCR5 and wildtype pigs or over time (FIGS. 14 S and T).

### REFERENCES

1. Russell P, Atkinson K, Krishnan L (1980) Recurrent reproductive failure due to severe placental villitis of unknown etiology. J Reprod Med 24: 93-98.
2. Wensvoort G, Terpstra C, Pol J M, ter Laak E A, Bloemraad M, et al. (1991) Mystery swine disease in The Netherlands: the isolation of Lelystad virus. Vet Q 13: 121-130.
3. Karniychuk U U, Saha D, Geldhof M, Vanhee M, Cornillie P, et al. (2011) Porcine reproductive and respiratory syndrome virus (PRRSV) causes apoptosis during its replication in fetal implantation sites. Microb Pathog 51: 194-202.
4. Pejsak Z, Stadejek T, Markowska-Daniel I (1997) Clinical signs and economic losses caused by porcine reproductive and respiratory syndrome virus in a large breeding farm. Vet Microbiol 55: 317-322.
5. Christianson W T, Joo H S (1994) Porcine reproductive and respiratory syndrome: A review. J

Swine Health Prod 2: 10-28.
6. Wagstrom E A, Chang C C, Yoon K J, Zimmerman J J (2001) Shedding of porcine reproductive and respiratory syndrome virus in mammary gland secretions of sows. Am J Vet Res 62: 1876-1880.
7. Moore C (1990) Clinical presentation of mystery swine disease in growing pig. Proc Mystery

Swine Dis Comm Meet Livest Consery Inst Denver, Colo.: 173-175.
8. White M E C (1992) The clinical signs and symptoms of blue-eared pig disease (PRRS). Pig Vet J 28: 62-68.
9. Holtkamp D J, Kliebenstein J B, Neumann E J, Zimmerman J J, Rotto H F, et al. (2013) Assessment of the economic impact of porcine reproductive and respiratory syndrome virus on United States pork producers. J Swine Health Prod 21: 72-84.
10. Neumann E J, Kliebenstein J B, Johnson C D, Mabry J W, Bush E J, et al. (2005) Assessment of the economic impact of porcine reproductive and respiratory syndrome on swine production in the United States. J Am Vet Med Assoc 227: 385-392.
11. Cavanagh D (1997) Nidovirales: a new order comprising Coronaviridae and Arteriviridae. Arch Virol 142: 629-633.
12. Meulenberg J J, Hulst M M, de Meijer E J, Moonen P L, den Besten A, et al. (1994) Lelystad virus belongs to a new virus family, comprising lactate dehydrogenase-elevating virus, equine arteritis virus, and simian hemorrhagic fever virus. Arch Virol Suppl 9: 441-448.
13. Wu W H, Fang Y, Farwell R, Steffen-Bien M, Rowland R R, et al. (2001) A 10-kDa structural protein of porcine reproductive and respiratory syndrome virus encoded by ORF2b. Virology 287: 183-191.
14. Meulenberg J J, Petersen-den Besten A (1996) Identification and characterization of a sixth structural protein of Lelystad virus: the glycoprotein GP2 encoded by ORF2 is incorporated in virus particles. Virology 225: 44-51.
15. Meulenberg J J, Petersen-den Besten A, De Kluyver E P, Moormann R J, Schaaper W M, et al. (1995) Characterization of proteins encoded by ORFs 2 to 7 of Lelystad virus. Virology 206: 155-163.
16. Wieringa R, de Vries A A, Rottier P J (2003) Formation of disulfide-linked complexes between the three minor envelope glycoproteins (GP2b, GP3, and GP4) of equine arteritis virus. J Virol 77: 6216-6226.
17. van Nieuwstadt A P, Meulenberg J J, van Essen-Zanbergen A, Petersen-den Besten A, Bende R J, et al. (1996) Proteins encoded by open reading frames 3 and 4 of the genome of Lelystad virus (Arteriviridae) are structural proteins of the virion. J Virol 70: 4767-4772.
18. Duan X, Nauwynck H J, Pensaert M B (1997) Virus quantification and identification of cellular targets in the lungs and lymphoid tissues of pigs at different time intervals after inoculation with porcine reproductive and respiratory syndrome virus (PRRSV). Vet Microbiol 56: 9-19.
19. Lawson S R, Rossow K D, Collins J E, Benfield D A, Rowland R R (1997) Porcine reproductive and respiratory syndrome virus infection of gnotobiotic pigs: sites of virus replication and co-localization with MAC-387 staining at 21 days post-infection. Virus Res 51: 105-113.
20. Teifke J P, Dauber M, Fichtner D, Lenk M, Polster U, et al. (2001) Detection of European porcine reproductive and respiratory syndrome virus in porcine alveolar macrophages by two-colour immunofluorescence and in-situ hybridization-immunohistochemistry double labelling. J Comp Pathol 124: 238-245.
21. Reiner G, Fresen C, Bronnert S, Willems H (2009) Porcine Reproductive and Respiratory Syndrome Virus (PRRSV) infection in wild boars. Vet Microbiol 136: 250-258.
22. Kreutz L C, Ackermann M R (1996) Porcine reproductive and respiratory syndrome virus enters cells through a low pH-dependent endocytic pathway. Virus Res 42: 137-147.
23. Nauwynck H J, Duan X, Favoreel H W, Van Oostveldt P, Pensaert M B (1999) Entry of porcine reproductive and respiratory syndrome virus into porcine alveolar macrophages via receptor-mediated endocytosis. J Gen Virol 80 (Pt 2): 297-305.
24. Shi C, Liu Y, Ding Y, Zhang Y, Zhang J (2015) PRRSV receptors and their roles in virus infection. Arch Microbiol 197: 503-512.
25. Delputte P L, Vanderheijden N, Nauwynck H J, Pensaert M B (2002) Involvement of the matrix protein in attachment of porcine reproductive and respiratory syndrome virus to a heparinlike receptor on porcine alveolar macrophages. J Virol 76: 4312-4320.
26. Jusa E R, Inaba Y, Kouno M, Hirose 0 (1997) Effect of heparin on infection of cells by porcine reproductive and respiratory syndrome virus. Am J Vet Res 58: 488-491.
27. Vanderheijden N, Delputte P, Nauwynck H, Pensaert M (2001) Effects of heparin on the entry of porcine reproductive and respiratory syndrome virus into alveolar macrophages. Adv Exp Med Biol 494: 683-689.
28. Wissink E H, van Wijk H A, Pol J M, Godeke G J, van Rijn P A, et al. (2003) Identification of porcine alveolar macrophage glycoproteins involved in infection of porcine respiratory and reproductive syndrome virus. Arch Virol 148: 177-187.
29. Vanderheijden N, Delputte P L, Favoreel H W, Vandekerckhove J, Van Damme J, et al. (2003) Involvement of sialoadhesin in entry of porcine reproductive and respiratory syndrome virus into porcine alveolar macrophages. J Virol 77: 8207-8215.
30. Prather R S, Rowland R R, Ewen C, Trible B, Kerrigan M, et al. (2013) An intact sialoadhesin (Sn/SIGLEC1/CD169) is not required for attachment/internalization of the porcine reproductive and respiratory syndrome virus. J Virol 87: 9538-9546.
31. Frydas I S, Verbeeck M, Cao J, Nauwynck H J (2013) Replication characteristics of porcine reproductive and respiratory syndrome virus (PRRSV) European subtype 1 (Lelystad) and subtype 3 (Lena) strains in nasal mucosa and cells of the monocytic lineage: indications for the use of new receptors of PRRSV (Lena). Vet Res 44: 73.
32. Law S K, Micklem K J, Shaw J M, Zhang X P, Dong Y, et al. (1993) A new macrophage differentiation antigen which is a member of the scavenger receptor superfamily. Eur J Immunol 23: 2320-2325.
33. Onofre G, Kolackova M, Jankovicova K, Krejsek J (2009) Scavenger receptor CD163 and its biological functions. Acta Medica (Hradec Kralove) 52: 57-61.
34. Van Gorp H, Delputte P L, Nauwynck H J (2010) Scavenger receptor CD163, a Jack-of-all-trades and potential target for cell-directed therapy. Mol Immunol 47: 1650-1660.
35. Calvert J G, Slade D E, Shields S L, Jolie R, Mannan R M, et al. (2007) CD163 expression confers susceptibility to porcine reproductive and respiratory syndrome viruses. J Virol 81: 7371-7379.
36. Van Gorp H, Van Breedam W, Delputte P L, Nauwynck H J (2008) Sialoadhesin and CD163 join forces during entry of the porcine reproductive and respiratory syndrome virus. J Gen Virol 89: 2943-2953.
37. Whitworth K M, Rowland R R, Ewen C L, Trible B R, Kerrigan M A, et al. (2016) Gene-edited pigs are protected from porcine reproductive and respiratory syndrome virus. Nat Biotechnol 34: 20-22.
38. Groenen M A, Archibald A L, Uenishi H, Tuggle C K, Takeuchi Y, et al. (2012) Analyses of pig genomes provide insight into porcine demography and evolution. Nature 491: 393-398.
39. Kallberg M, Wang H, Wang S, Peng J, Wang Z, et al. (2012) Template-based protein structure modeling using the RaptorX web server. Nat Protoc 7: 1511-1522.
40. Cong L, Ran F A, Cox D, Lin S, Barretto R, et al. (2013) Multiplex genome engineering using CRISPR/Cas systems. Science 339: 819-823.
41. Jinek M, Chylinski K, Fonfara I, Hauer M, Doudna J A, et al. (2012) A programmable dual-RNA-guided DNA endonuclease in adaptive bacterial immunity. Science 337: 816-821.
42. Chen B, Gilbert L A, Cimini B A, Schnitzbauer J, Zhang W, et al. (2013) Dynamic imaging of genomic loci in living human cells by an optimized CRISPR/Cas system. Cell 155: 1479-1491.
43. Komohara Y, Hirahara J, Horikawa T, Kawamura K, Kiyota E, et al. (2006) AM-3K, an anti-macrophage antibody, recognizes CD163, a molecule associated with an anti-inflammatory macrophage phenotype. J Histochem Cytochem 54: 763-771.
44. Ziegler-Heitbrock H W, Fingerle G, Strobel M, Schraut W, Stelter F, et al. (1993) The novel subset of CD14+/CD16+ blood monocytes exhibits features of tissue macrophages. Eur J Immunol 23: 2053-2058.
45. Kapetanovic R, Fairbairn L, Downing A, Beraldi D, Sester D P, et al. (2013) The impact of breed and tissue compartment on the response of pig macrophages to lipopolysaccharide. BMC Genomics 14: 581.
46. Fairbairn L, Kapetanovic R, Beraldi D, Sester D P, Tuggle C K, et al. (2013) Comparative analysis of monocyte subsets in the pig. J Immunol 190: 6389-6396.
47. Hartnell A, Steel J, Turley H, Jones M, Jackson D G, et al. (2001) Characterization of human sialoadhesin, a sialic acid binding receptor expressed by resident and inflammatory macrophage populations. Blood 97: 288-296.
48. Sanchez C, Domenech N, Vazquez J, Alonso F, Ezquerra A, et al. (1999) The porcine 2A10 antigen is homologous to human CD163 and related to macrophage differentiation. J Immunol 162: 5230-5237.
49. Piriou-Guzylack L, Salmon H (2008) Membrane markers of the immune cells in swine: an update. Vet Res 39: 54.
50. Kappes M A, Faaberg K S (2015) PRRSV structure, replication and recombination: Origin of phenotype and genotype diversity. Virology 479-480: 475-486.
51. Stadejek T, Oleksiewicz M B, Scherbakov A V, Timina A M, Krabbe J S, et al. (2008) Definition of subtypes in the European genotype of porcine reproductive and respiratory syndrome virus: nucleocapsid characteristics and geographical distribution in Europe. Arch Virol 153: 1479-1488.
52. Edwards S (1992) PRRSV (“blue-eared pig disease”) in Great Birtain. American Association of Swine Practitionsers Newsletter 4: 32-36.
53. Stadejek T, Stankevicius A, Storgaard T, Oleksiewicz M B, Belak S, et al. (2002) Identification of radically different variants of porcine reproductive and respiratory syndrome virus in Eastern Europe: towards a common ancestor for European and American viruses. J Gen Virol 83: 1861-1873.
54. Morgan S B, Graham S P, Salguero F J, Sanchez Cordon P J, Mokhtar H, et al. (2013) Increased pathogenicity of European porcine reproductive and respiratory syndrome virus is associated with enhanced adaptive responses and viral clearance. Vet Microbiol 163: 13-22.
55. Basta S, Knoetig S M, Spagnuolo-Weaver M, Allan G, McCullough K C (1999) Modulation of monocytic cell activity and virus susceptibility during differentiation into macrophages. J Immunol 162: 3961-3969.
56. Shanmukhappa K, Kim J K, Kapil S (2007) Role of CD151, A tetraspanin, in porcine reproductive and respiratory syndrome virus infection. Virol J 4: 62.
57. Boyer L C, Cardo-Vila M, Kuniyasu A, Sun J, Rangel R, et al. (2007) A previously unrecognized protein-protein interaction between TWEAK and CD163: potential biological implications. J Immunol 178: 8183-8194.
58. Fabriek B O, van Bruggen R, Deng D M, Ligtenberg A J, Nazmi K, et al. (2009) The macrophage scavenger receptor CD163 functions as an innate immune sensor for bacteria. Blood 113: 887-892.
59. Ugocsai P, Barlage S, Dada A, Schmitz G (2006) Regulation of surface CD163 expression and cellular effects of receptor mediated hemoglobin-haptoglobin uptake on human monocytes and macrophages. Cytometry A 69: 203-205.
60. Schaer D J, Schaer C A, Buehler P W, Boykins R A, Schoedon G, et al. (2006) CD163 is the macrophage scavenger receptor for native and chemically modified hemoglobins in the absence of haptoglobin. Blood 107: 373-380.
61. Snijder E J, Kikkert M, Fang Y (2013) Arterivirus molecular biology and pathogenesis. J Gen Virol 94: 2141-2163.
62. Hashimoto M, Takemoto T (2015) Electroporation enables the efficient mRNA delivery into the mouse zygotes and facilitates CRISPR/Cas9-based genome editing. Sci Rep 5: 11315.
63. Kaneko T, Sakuma T, Yamamoto T, Mashimo T (2014) Simple knockout by electroporation of engineered endonucleases into intact rat embryos. Sci Rep 4: 6382.
64. Qin W, Dion S L, Kutny P M, Zhang Y, Cheng A W, et al. (2015) Efficient CRISPR/Cas9-Mediated Genome Editing in Mice by Zygote Electroporation of Nuclease. Genetics 200: 423-430.
65. Takahashi G, Gurumurthy C B, Wada K, Miura H, Sato M, et al. (2015) GONAD: Genome-editing via Oviductal Nucleic Acids Delivery system: a novel microinjection independent genome engineering method in mice. Sci Rep 5: 11406.
66. Sato M, Ohtsuka M, Watanabe S, Gurumurthy C B (2016) Nucleic acids delivery methods for genome editing in zygotes and embryos: the old, the new, and the old-new. Biol Direct 11: 16.
67. Lillico S G, Proudfoot C, Carlson D F, Stverakova D, Neil C, et al. (2013) Live pigs produced from genome edited zygotes. Sci Rep 3: 2847.
68. Whitworth K M, Lee K, Benne J A, Beaton B P, Spate L D, et al. (2014) Use of the CRISPR/Cas9 system to produce genetically engineered pigs from in vitro-derived oocytes and embryos. Biol Reprod 91: 78.
69. Sato M, Koriyama M, Watanabe S, Ohtsuka M, Sakurai T, et al. (2015) Direct Injection of CRISPR/Cas9-Related mRNA into Cytoplasm of Parthenogenetically Activated Porcine Oocytes Causes Frequent Mosaicism for Indel Mutations. Int J Mol Sci 16: 17838-17856.
70. Chen S, Lee B, Lee A Y, Modzelewski A J, He L (2016) Highly Efficient Mouse Genome Editing by CRISPR Ribonucleoprotein Electroporation of Zygotes. J Biol Chem 291: 14457-14467.
71. Chen J M, Cooper D N, Chuzhanova N, Ferec C, Patrinos G P (2007) Gene conversion:

mechanisms, evolution and human disease. Nat Rev Genet 8: 762-775.
72. Ran F A, Hsu P D, Wright J, Agarwala V, Scott D A, et al. (2013) Genome engineering using the CRISPR-Cas9 system. Nat Protoc 8: 2281-2308.
73. Lillico S G, Proudfoot C, King T J, Tan W, Zhang L, et al. (2016) Mammalian interspecies substitution of immune modulatory alleles by genome editing. Sci Rep 6: 21645.
74. Li Y, Tas A, Snijder E J, Fang Y (2012) Identification of porcine reproductive and respiratory syndrome virus ORF1a-encoded non-structural proteins in virus-infected cells. J Gen Virol 93: 829-839.
75. Burkard C, Lillico S G, Reid E, Jackson B, Mileham A J, Ait-Ali T, Whitelaw C B, Archibald A L. 2017. Precision engineering for PRRSV resistance in pigs: Macrophages from genome edited pigs lacking CD163 SRCR5 domain are fully resistant to both PRRSV genotypes while maintaining biological function. PLoS Pathog 13:e1006206.
76. Kulkarni M, Bowman E, Gabriel J, Amburgy T, Mayne E, Zidar D A, Maierhofer C, Turner A N, Bazan J A, Koletar S L, Lederman M M, Sieg S F, Funderburg N T. 2016. Altered Monocyte and Endothelial Cell Adhesion Molecule Expression Is Linked to Vascular Inflammation in Human Immunodeficiency Virus Infection. Open Forum Infect Dis 3:ofw224.
77. Rappe J C, Garcia-Nicolas O, Fluckiger F, Thur B, Hofmann M A, Summerfield A, Ruggli N. 2016. Heterogeneous antigenic properties of the porcine reproductive and respiratory syndrome virus nucleocapsid. Vet Res 47:117.
78. Halbur P G, Paul P S, Frey M L, Landgraf J, Eernisse K, Meng X J, Lum M A, Andrews J J, Rathje J A. 1995. Comparison of the pathogenicity of two US porcine reproductive and respiratory syndrome virus isolates with that of the Lelystad virus. Vet Pathol 32:648-660.
79. Halbur P G, Andrews J J, Huffman E L, Paul P S, Meng X J, Niyo Y. 1994. Development of a streptavidin-biotin immunoperoxidase procedure for the detection of porcine reproductive and
80. respiratory syndrome virus antigen in porcine lung. J Vet Diagn Invest 6:254-257. Arango Duque G, Descoteaux A. 2014. Macrophage cytokines: involvement in immunity and infectious diseases. Frontiers in immunology 5:491.
81. Zhang J M, An J. 2007. Cytokines, inflammation, and pain. International anesthesiology clinics 45:27-37.

Nucleic Acid Sequences:

CD163 Guide Sequences:

sgSL25
(SEQ ID NO: 5)
TGAAAAATAGCATTTCGGTG
CD163 gene cut location:
(SEQ ID NO: 6)
CAC|CGAAATGCTATTTTTCA
sgSL26
(SEQ ID NO: 7)
GAATCGGCTAAGCCCACTGT
CD163 gene cut location:
(SEQ ID NO: 8)
GAATCGGCTAAGCCCAC|TGT
sgSL27
(SEQ ID NO: 9)
GTCCTCCATTTACTGTAATC
CD163 gene cut location:
(SEQ ID NO: 10)
GAT|TACAGTAAATGGAGGAC
sgSL28
(SEQ ID NO: 11)
CCCATGCCATGAAGAGGGTA
CD163 gene cut location:
(SEQ ID NO: 11)
CCCATGCCATGAAGAGG|GTA
Cut locations are shown by the | symbol.

Genomic Sequence of the CD163 Gene Locus in Large White Pigs (SEQ ID NO 1)

Bold=exons

Single underlined and dashed underline=splice acceptor site predictions

Double underlined=splice donor site predictions

sgRNA binding locations and cutting sites are indicated in lowercase italics, and the particular sgRNA binding to the sites is also indicated.

| | |
|---|---|
| 1 | TCTTCATCCT ATTAGAGACA CTGCTATACA GCAGAAATTG ACACAACATT GTAAATCAAC |
| 61 | TATACTTTAA TAAAATAAAA AAAAGAAATA CAAGTGCTTT CTACAGACAA TCTGCACAAG |
| 121 | TTATTTGTTA GACATATTTG ATTATAGAAT TAATATTAAA AGGGGTTATA ACAATCAAGC |
| 181 | ATTGATAATT TAATTATGTT TGCCTATTTT ACTTTAGTTT TTTGACATAA CTGTGTAACT |
| 241 | ATTGCGATTT TTTTATTCCT AATGTAATTA GTTCAAAACA AAGTGCAGAA ATTTAAAATA |
| 301 | TTCAATTCAA CAACAGTATA TAAGTCAATA TTCCCCCCTT AAATTTTTAC AAATCTTTAG |
| 361 | GGAGTGTTTC TCAATTTCTC AATTTCTTTG GTTGTTTCAT GTCCCATATG GAAGAAAACA |
| 421 | TGGGTGTGAA AGGGAAGCTT ACTCTTTTGA TTACTTCCCT TTTCTGGTTG ACTCCACCTC |
| 481 | CATTATGAAG CCTTTCTGTA TTTTTGTGGA AGTGAAATGA TTTTTAGAAT TCTTAGTGGT |
| 541 | TCTCTTCTTC AGGAGAACAT TTCTAGGTAA TAATACAAGA AGATTTAAAT GGCATAAAAC |
| 601 | CTTGGAATGG ACAAACTCAG AATGGTGCTA CATGAAAACT CTGGATCTGC AGGTAAAATC |
| 661 | TTCTCATTTA TTCTATATTT ACCTTTTAAT AGAGTGTAGC AATATTCCGA CAGTCAATCA |
| 721 | ATCTGATTTA ATAGTGATTG GCATCTGGAG AAGAAGTAAC AGGGAAAAGG CAATAAGCTT |
| 781 | ATAAGGGGAA CTTTTATCTT CCATAGAATC AAAATTGAAG ACGTGACTAG AAGAAGGATT |
| 841 | AGATTTGGCA TCAGTTTTGT AAAATTGCTG AGGTGAAATT AAGTAAGGGA TGAAAATTAA |
| 901 | CTAAATTGTG TTGAGTATGA AACTAGTAGT TGTTAGAAAA GATAGAACAT GAAGGAATGA |
| 961 | ATATTGATTG AAAGTTGATG ACCTAGAGGA CATTTAGACT AACACCTCTG AGTGTCAAAG |
| 1021 | TCTAATTTAT GATTTACATC GATGCGTTAA ACTCATTTAA CATTCTTACT TTTTTCCCCT |
| 1081 | CAAGCATTTA AGCTGAAGTA TAACATTTCA CATGAAAGCC TGGATTATAA ATGCACAGTT |
| 1141 | CAGTGACCTA TCTCAGAGGA GTGACTGCCA TAGCATTTTT TTTGTCTTTT TGCCTTCAGA |
| 1201 | GCCACAGCAA CGCGGGATCC GAAGCCGCGT CTGCGACCCA CACCACAGCT CACGGCAATG |
| 1261 | CCGGATCTTT AACCCACTGA GCGAGGCCGG GGATCGAACC CGCAGTCTCA TGGTTCCTAG |
| 1321 | TAGGATTCGT TAACCACTGC GCCACGACGG GAACTCCTAC CATAGCATTT TTACTTTTAA |
| 1381 | GTTACTGTTG GTTTAGAGTA AGAAGGAGAA ATGAGAGTGA TGGAGCGTTT GCTATATTTG |
| 1441 | GAGACAAGGT CCTATATTGG AGGTTCTCAA ATATAAATTT TGTCGCTTTT TCCTCCAATG |
| 1501 | TATTGTTCAA CTACTATTTA GCAGGCCACT GTGCCAGGTA CTGGTGAAAC TGGTGAACAT |
| 1561 | GATAGATGTA ATTCATTCCC TCATGGAACT TTCCATCTAA CAATGTGGAT CAGGTAGGCT |
| 1621 | TGGAGATGAG AATGCCAGTG GTTGACTATG ACTCTGTGGC TGAAGGGAGA GCTACTCACT |
| 1681 | TCGTAGTTTC ATCAATGTCT TTTTGGTTTT CCAGGTTTTA AGCCCTGCTC TTGCAATTCT |
| 1741 | TTTCCCTTCT CCAACTTTCT TCTAATTTCT CACCCCTAGG ATGCCTATAA ACATGAGTAT |
| 1801 | TTTCAAAGCT ACTTCACTGA GGTTATATGA TCCTCGTGTG AATTTTTCCT GCCTGCCTTG |
| 1861 | CCATTTAGAA GGAAGTGTTT CCTGGAATTT CCATTGTGGC TTGGTGGTTA AAGACCCTGC |
| 1921 | ATTGTCTCTG TGAGGATGTG GGTTCAATCT CTGGCCTCAT TCAGTGAGTG GGTTAAGGAT |
| 1981 | CTGGTGTCGC TGCAAGCTGT GGCTAAGATC CCACATTGCC ATGGCTGTGG TGTAGACTGG |
| 2041 | CACCTGGAGC TCTGATTTGA CCACAATCCT AGGAACTTCA GATGTTGCCA TAAAAAGAAA |
| 2101 | AAAAAAGTTA GGAAGGGTTT TCTGTCTTGT TTTGACCTTT GTTAATCTCA AACCTTTGGA |
| 2161 | ACCATCTCTC CTCCAAAACC TCCTTTGGGT AAGACTGTAT GTTTGCCCTC TCTCTTCTTT |
| 2221 | TCGCAGACTT TAGAAGATGT TCTGCCCATT TAAGTTCCTT CACTTTTGCT GTAGTCGCTG |
| 2281 | TTCTCAGTGC CTGCTTGGTC ACTAGTTCTC TTGGTGAGTA CTTTGACAAA TTTACTTGTA |
| 2341 | ACCTAGCCCA CTGTGACAAG AAACACTGAA AAGCAAATAA TTCTCCTGAA GTCTAGATAG |
| 2401 | CATCTAAAAA CATGCTTCAT GGTTTCAAAG GATCAGATAT TAAAAACCCC AAATAGGTAC |
| 2461 | AGAACCATGT GGCTCTCTCC CCCCAAACAA ATAAAACGTT AGCATGGTTT TCAAAAAAAT |
| 2521 | AAAATAACCT TCACAGGAAA AATGGATTTT ACTTAAGATT TGAAATAATA TCTAACTAAA |
| 2581 | AAATAGGGAA TAATGCAGAA GAGGAGAAAC CTCAGAATTG TTGGGATGAA GGAATTTTTA |
| 2641 | GTAACACTAA AAATTCAAGT GCCAAAATTT GTCTAAAATT GTATTCAGGG AAGCCAGATA |
| 2701 | TATATCAGTG AAATCGCCAG TTCCTATATT AGCTAAAATA ATCACAAGGC TGTAGCAGAG |
| 2761 | ACAGTTCAGA GAGAGGTGGA GATGAGATTT TTTTTTTTTA AGTATAATTG ATTTACAATG |
| 2821 | TTGTGGCAAT TTCTGTTGTA TAGCAAGAGA TAGAATTATT TTATGGTGGA AGATAATAGA |
| 2881 | AAAATATATC CATATCAATT TCCATTTGAG TAGATAAATT TCAATTAGAG TTCAACTAGC |
| 2941 | AATTAGTAGT TTTGCATACA TGGTGAAATA TATTCATGGT ATTTTGCATA TATGTGTGAA |
| 3001 | ATAGGTACTA AATTCCTCAT AACTGTTCTT TTTAGTCTCA CCATCAGCCT CTACTGATCT |
| 3061 | TAGGATTTTG GAGAAACATA CATAGTTCAT CCCTATAAAA TGCCATAAAA TCTCATTTTT |
| 3121 | ACATTAAACC ATCCAAGAGA TTATATAAAT TGACCTTATA AAGAATATCA GCCATAAAAT |
| 3181 | AAAGGTATCA TAGTATGGGA TTATTTAGCT TTATTGGTTC TATGTCACTG CTTAATTTGA |
| 3241 | AACCTGTGAT ATTGCTGTTT GTTTTTGAAC TCCTATGAAA TAACATTCTC CCATTGTACC |
| 3301 | ATGGATGGGT CCAGAAACAT TTCTCAAATC TGGCTTTGAA AAATAAATAA GTAATCTAAA |
| 3361 | GAATAATAAT TCTCTACTTG CTCTTTGAAT CTTGACCAAT TGCTGCATTT ACCTATTGTT |
| 3421 | ACAGGAGGAA AAGACAAGGA GCTGAGGCTA ACGGGTGGTG AAAACAAGTG CTCTGGAAGA |
| 3481 | GTGGAGGTGA AAGTGCAGGA GGAGTGGGGA ACTGTGTGTA ATAATGGCTG GGACATGGAT |
| 3541 | GTGGTCTCTG TTGTTTGTAG GCAGCTGGGA TGTCCAACTG CTATCAAAGC CACTGGATGG |
| 3601 | GCTAATTTTA GTGCAGGTTC TGGACGCATT TGGATGGATC ATGTTTCTTG TCGAGGGAAT |
| 3661 | GAGTCAGCTC TCTGGGACTG CAAACATGAT GGATGGGGAA AGCATAACTG TACTCACCAA |
| 3721 | CAGGATGCTG GAGTAACCTG CTCAGGTAAG ACATACACAA ATAAGTCAAG CCTATACATG |
| 3781 | AAATGCTTTG TGGGAAAAAA TGTATAGATG AGTTAAAAAC AAAAAGGAAC CAGTTTTCTA |
| 3841 | TAAGTCATCT AGTCCATGTA TAAAATTACC CAATCCATTA CTAAAAGACC ACTTCTGGTA |
| 3901 | TTTTACACAT GACAAAGCCC ATATTAAAAA AAAAAAATTC AGAAGAGATT CTGAATGCTA |
| 3961 | TAATAAATGA GCAAGTGACT AGCTTCAATT TTATATTAGG TCATTCTACC TTCTACTTCT |
| 4021 | ACATGAAAAT ATCATAATGT CTAAGTTAAT TCCTTGTCCC CTTTCCCAAT AAAGCACTGC |
| 4081 | TTTCATGCAC TGGCCTATGA ATCATGAACT TTTTGCCCTT TAACTGATGA TCAACTTACC |
| 4141 | AAATCAAGAA ATAAATATTC TTAGCACTGA TCCTTTTTTG TTGTTGTTGG AGGAAGAATG |
| 4201 | TTTTGCAAAG TAGAATTGCT TTTTTCTGTT TAACAGTGCT ATTCATTTCA TTTACATGGT |
| 4261 | CGTTTTAATT TATAAAACAT TTCATAAGTT TCACCTCATA TGCCCTTACA ATAACTCAGG |
| 4321 | AAGTTATATG TTAGACCTTT CTGCTGACAA ATCCCAGAGT CATGTTTCTG ACCCAGTTCA |
| 4381 | GATTCCTTGG CTTCCCATTT CTCTTTGCTC ATGTCATTGA CCTTTATGCA GCCCTCTTAC |
| 4441 | CTCCCACCTT TCTATTACAG ACCATCTCCT CCATAGGACT GGTGTTAGAA AGTACTAATC |
| 4501 | TCTACCCAGG CATTGTGGTG CAATGTGGGC AGCACAGGCT GGTATCTAGA AAAATGCTGA |
| 4561 | AGTGAATTCC AGCTCAGCTG CTCGTTAATA CTATTGTTTT AAGTAAGCTG TTCAATCCTT |
| 4621 | TGAAATTCAC TTTCTGAGCA CTCAGTGATA TAATAAATGT AGAGTTACTG GTACACTGTC |
| 4681 | TGGTATGTAA TAGGTGTTAA AAATTAACCT TAGTTTCCTC ATGGGTCACT GCTTCTCATT |
| 4741 | ACCTAGACAA CTCATTTCTC TTTCTTCCTC TTTCTCTTTC TCCATTCTCC TCCTCCTTCT |
| 4801 | TCCTCTTCTT CTTGTCTTTT ATTGTTATTC ATTTTGCTGA GAAAGTTAAG AAATAACAAC |
| 4861 | TCTAACCTCT ACATCGACCA CCTAGAGCAA AGTTAAAAAT AATAATAAAC CTTGCCAGAC |
| 4921 | TCTTACTATA ATTGTTGCTG TCTATAGAGT TGACTGTTTA AGTTAAGACA TCAGTATAGT |
| 4981 | ATTTTTAATT TTTGTGTTTT TTTTTTCATA CTTTTACATG AGGATCCTTT ATATAAGGAT |
| 5041 | GAGTTAAACA AACTTGATTT TTGAAGTTTA TACCCCTGAG GCTCAACTGC ATAATAATAG |
| 5101 | AAAGGGATCC ATAGCCTCTC AAGGACTTAA CTAGTTTCAT GAGTTTTCAG AATCTGAATT |
| 5161 | TCTGAGATTC TCCACCCCAA TTAAAGCTCA AGCCTCAGAA CATATATCCT TCTCTTGGTA |
| 5221 | AATTCTATTC TTATCACATG CGTAATAATA AAAAAGAGAG ATGTTGGAGA CAGATTTTTT |
| 5281 | TCCTCACATT CTGTCTCTAC TGTTTTCTAG GTGTTTGATT CTGTGTTATT TAACCTCAGT |
| 5341 | TTGCTTATCT GTGAAGTAGG GATTATGGTA ATAACATATA ATGCTTAATG TTGTAAAGAC |
| 5401 | TAAAGAAGAT AGCATATGTA ACACATTTGG AACAGGGAAT GCATATTTTG ATTGTGAGCT |
| 5461 | CTTATTATTA TTACCAATCA GCCATAATAA AAATCTTGTT AAGTGGAGGT CTTTGGATTT |
| 5521 | CAGAGCTTTT AAAATCTAAT TACTTTTTCA AAAAAGAGCT TCTTAGTGTT TTTTTTTTTT |
| 5581 | AACCACAAAG TGTTTCTATT TTTTAGGTGT CCCAAAATTT CATTCCAAAT ATCTTTTTCT |
| 5641 | CAGATATTTT AGTCCTCATA GAACACCTAG GGATAGTGTA TAGAGAAAAT TTTCTTTATT |
| 5701 | AAAAAGCTGT TCTTTGCTAA AAATTGTAGC AGGTACTTTT GGGAGGGGGG AAAACTTTGA |
| 5761 | TTCAGAAACT GCTAAGACAT GGAGTGTTTT GACTAATTTT TCCTCAATTT TTAATGTTTT |
| 5821 | TTATACCATA GGGTACTTTT GCAAACTATT ATGCATACTT ATATATTTTT ACTTTTTTCC |
| 5881 | TGTCTTTTAA CTTCCAAATT CAACTTCAGA CAATTATTCA TGCACTAAAC TGTTGTAGTA |
| 5941 | AGAAAGATTA AAATTAAAAA ATTAACCATT CAACAAATGA CTGGTTTGCC ATTTTTACTA |
| 6001 | CTTTGTTGTA TGAACAATTT TTTTTTCTAC AAATGAATAC TTTGAGTCTG ATTTATCCAT |
| 6061 | TCCTACATAA AAGTTTTTAC TATATCTTAG TATTGGAAGG AAACAAAACA AAACACAATG |
| 6121 | TAAATTTTAA TCTATAAATT TTGGGGGGGG GTAAATATAC ATAGATGAAA GTCTTAACCA |
| 6181 | TTAATTAGAG TCAAAAGATT AAAATTCTCC AATATGTGAA CTTAGGCTGC ATCCAAAATG |
| 6241 | AAGCATCATT TTTAAGGACA GCATCAAAAG TGACCAGAGG AATTTTACTT TCTTTCTTTT |
| 6301 | TTTTTTTTTT TTTGAATTTT AGTTTCTAAA CTCACTTCTG AATAAATACA ACTTCTAAAT |
| 6361 | TCTCGTCTTT TCTCTACTCT AGATGGATCT GATTTAGAGA TGAGGCTGGT GAATGGAGGA |
| 6421 | AACCGGTGCT TAGGAAGAAT AGAAGTCAAA TTTCAAGGAC GGTGGGGAAC AGTGTGTGAT |
| 6481 | GATAACTTCA ACATAAATCA TGCTTCTGTG GTTTGTAAAC AACTTGAATG TGGAAGTGCT |
| 6541 | GTCAGTTTCT CTGGTTCAGC TAATTTTGGA GAAGGTTCTG GACCAATCTG GTTTGATGAT |
| 6601 | CTTGTATGCA ATGGAAATGA GTCAGCTCTC TGGAACTGCA AACATGAAGG ATGGGGAAAG |
| 6661 | CACAATTGCG ATCATGCTGA GGATGCTGGA GTGATTTGCT TAAGTAAGGA CTGACCTGGG |
| 6721 | TTTGTTCTGT TCTCCATGAG AGGGCAAAAA AAGGGGAGTA AAAGTCTTAA AAGCTCAAAC |
| 6781 | TGTTAAAAAC ATAATGATGA TTGCTTCTTT TATCATCTTA TTATTATCTA ATTTCAGGTC |
| 6841 | GAAATTCTAG TACCTGTGCA GTTTTTTACC TTAACTGAAA TTAAGATAAA TAGGATAGGG |
| 6901 | AGGAAGGATG AGCAGTGACA TTTAGGTCCA AGTCATGAGG TTAGAAGGAA ATGTTCAGAG |
| 6961 | AATAGCCCAT TCCCTCAGCC CTCAAAGAAA GAAAGAAAGA AAAAGAAAAA AAAAAAGAAA |
| 7021 | GCTTAACTAG AAAATTTTGT TCTCTGGATG TTTTAGAGGC AAACCATCCC TTTTATCATT |
| 7081 | CCTTACCTAC AAAGCCCTTC TCTTTAATCA CATTGACCCA CCCTTTCCTA AACTATTAGT |
| 7141 | TCAAATTCAC ATAATTGAAT GCTTTTAAAA CTTGGTTTCC TCTTATAATT ATATTTATGT |
| 7201 | TGTAAGGAGG CACTGTGTCT TGTCTAGAGA CTTTCATGTT CTATGCTTGA TTATGGGACA |
| 7261 | GGGACATGGC TTTGTCTGCT CCAGGATGTC ACTCTCCTTT TTTCACTTGA GCTCCTAGTT |
| 7321 | TGAAGAAGAC CTAGTAAGTC TTGAACTCCA GGGAGTCTTT AGGAAACTAT CCCTAGAGCA |
| 7381 | AAACTGTCCC TGAATTCACC CAGTGTCTTT TTTTTTTTTT TCAAATGAAG GAACTTTAGT |
| 7441 | TCAAACTAAA TTTAAAATAA GGGAATTCTA ATTCAGAATA CTGGGAAATC CAGGAGATTA |
| 7501 | CAATTGGCTT CATGTGTGAT TGGATTCAGC ACTTCACCAA TGTCATCAGG GTTCTGGTTC |
| 7561 | TTTTTTTATT TCTTGAATTG GCTTTTTTTT TTTTTTCCTT GTTGAACAAT ATGACTATCT |
| 7621 | ATACTTTGAA CCACAAAGAA AGTGATTCCT ACAGAAAAGA CAGAATGTGT TAGCTGAAGG |
| 7681 | AAGGGAATGG GACTTGGGGT AGAAAAAAAC ACCTTCCGTA TTCCTTAACC TATCAAAAAT |
| 7741 | TTCTAGGTAC CCCTAACTAA AATCCTAATT CAAGCATATT GGAGGAACTT GACAAATCCA |
| 7801 | GGAATAATAT TATCCGTTAT CAAATACATG CACATCATTT ACATTTCTCC ATGTCTCTGC |
| 7861 | TCATGCAGTT CCCGGCCCTA ACTCTACCAA AGTATTACTC TCCATCTCCC TCTTTTTTTT |
| 7921 | TTTAATGATT TTTATTTTTT CTGTTATGAC TGGTTTACAG TGTTCTGTCA ATTTTCTACT |
| 7981 | GTACAGCAAA GTGACCCAGT CACACATTCA TATATACATT CTTTTTCTCA CATTATCCTC |
| 8041 | CATCAGGCTC CATCACAAGT GACTAGACAT AGTTCCCAGA GCTATGCAGC AGGATCTCAT |
| 8101 | TGCTGCTCCA TTCCAAAGGC AACAGTTCAC ATCTATTAAC CCCAGATTCC CAGTCCACCC |
| 8161 | CACTCCCTTC CCCTCCCTCT TGGCAACCAC AAGTCTGTTC TCCAAGTTCA TGAGTTTATT |
| 8221 | TTCTGTGGAA AGTTTTATTT GTGCAGTATG TTAGATTCCA GATATAAGTG CTATCATATG |
| 8281 | GTATTTGTCC TTCTCTTTCT GACTGACTTC ACAAAGTATG AGAGTCTCTA GTTCCATCCA |
| 8341 | TGTTACTGCA AATGGCATTA TTAATCTCCA TCTTTTTTTG TTCATGTATA TGTTACCCAG |
| 8401 | ATTCCTTGAC TTTTCTACAT CATCAAGATA TTGTTGATCA CTTCTTTGTA GTGATTTCTG |
| 8461 | CCCTTCTCTG ATGTCCTGTG ACACTAGTCT GGATTATTCA TTTACCTGAA ACCACATGTC |
| 8521 | TCTTATAATG TGTATCCCAA ATTAAATATG TCTATTGTAA TGTGTATCCC AAATTAAATA |
| 8581 | TTTATCTTTC TAAAAAAAAA AATTTCTAGG CCCCCAATCA GCATGTTTCT TCTCAGTGTG |
| 8641 | TTTTATACAT GCTGCAGAAT CATAATAGAC AGCATAATAG ACAGCATAAC AAAAACTAAA |
| 8701 | AATGCCAGGG GAAAAAAGCA ATTTACTGAT TACAACATAT TACTCAGAAT CAAGTTCTGT |
| 8761 | TCTTTGAGGA ATATTGATTG GGGGAAAATG AAAATAATGA TGGGGAGGTC CCTTTTCTCT |
| 8821 | TTGCTTTGCT TTTAAACTAC GGAAGTAGTC AGAAAGGGGT CAGGAATGTA ATATAAACCA |
| 8881 | GGTAGTCCTG GTAGGTAACG CAGCCGGAGG CAAAAGTGAG TGTTGAGTAT TGAGGCAAAC |
| 8941 | TGGAGGGCAT GGATACCACC TAGACAGATG CAAATATATA TTTAACAGGG AAAAAAGAAC |
| 9001 | CAAACAATTT CAACAAAAAA CCAAACAATT CCAACAAAAT TGGTCCAATA AGCAAACCTC |
| 9061 | TAGATAAATT TCAGTCCCTG GATGTTTTGT TAGGAACTCT TCCTACAATG CGTGCTTTCC |
| 9121 | ATTCTGAAAA GTCCTATCTA CTTGCCTGAT CCACTTCTCC TTCCATCCTA AACGATTTTC |
| 9181 | AGTGGTAGTA TATTACTGTT GTCTCTGTCT CTACTTATAT ATCTTCCCCT TTTCACTCAC |
| 9241 | TCCTCTCAGG TACAGCTCTT CAGTTTGCCC TTATTCTTGT TTCCTTGTCA ATGACTTGTT |
| 9301 | TTGTGTCCCT CTTACAGATG GAGCAGACCT GAAACTGAGA GTGGTAGATG GAGTCACTGA |
| 9361 | ATGTTCAGGA AGATTGGAAG TGAAATTCCA AGGAGAATGG GGAACAATCT GTGATGATGG |
| 9421 | CTGGGATAGT GATGATGCCG CTGTGGCATG TAAGCAACTG GGATGTCCAA CTGCTGTCAC |
| 9481 | TGCCATTGGT CGAGTTAACG CCAGTGAGGG AACTGGACAC ATTTGGCTTG ACAGTGTTTC |
| 9541 | TTGCCATGGA CACGAGTCTG CTCTCTGGCA GTGTAGACAC CATGAATGGG GAAAGCATTA |
| 9601 | TTGCAATCAT AATGAAGATG CTGGTGTGAC ATGTTCTGGT AAGTGAAAAC AAAACACCGG |
| 9661 | AAGGACCTGT GTTCTTCAGG ATTAGGAATG GATATGAGAT AGGAGAAAAA TTGTATCTAA |
| 9721 | TATTTTCTTT GTTGGGAATT CTTTTACAGT TGTGACAAAT CTTTAACATA TTCTTCATTT |
| 9781 | GAGTAGTTTG GAGGGTTGTC TGACTGTTTT CTATAATAAA TGTCCCAAGT GCTATGAGGT |
| 9841 | ACCACATTTC AAATTCTAAT TCTACCTGAA GCTCCAAAAA GACAAAATGT TATAGGTCTT |
| 9901 | TTCTTTATAT CTAATTTGCT TATGGTTTTT AGCCATTGAC AATTTTTTTT TTCTTAACTC |
| 9961 | TTGAAACTAT AATCCTATTT CTAACCAAAT TCATGTTCTA TACTGGCTCT TCAAAAACCC |
| 10021 | AGGAGATGGG AAAGCCAGAA TCTCCAGTGT TTCAGCTTCT GGGAAGGAGC AAGTTTTTAA |
| 10081 | 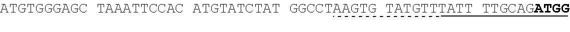 |
| 10141 | 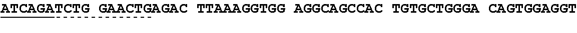 |
| 10201 | GGAAATTCAG AAACTGGTAG GAAAAGTGTG TGATAGAAGC TGGGGACTGA AAGAAGCTGA |
| 10261 | TGTGGTTTGC AGGCAGCTGG GATGTGGATC TGCACTCAAA ACATCATATC AAGTTTATTC |
| 10321 | CAAAACCAAG GCAACAAACA CATGGCTGTT TGTAAGCAGC TGTAATGGAA ATGAAACTTC |
| 10381 | TCTTTGGGAC TGCAAGAATT GGCAGTGGGG TGGACTTAGT TGTGATCACT ATGACGAAGC |
| 10441 | CAAAATTACC TGCTCAGGTA AGAATTTCAA TCAATGTGTT AGGAAATTGC ATTCTACTTT |
| 10501 | CTTTTACATG TAGCTGTCCA GTTTTCCCAG CACCACTTGT TGAAGAGACT GTCTTTTCTT |
| 10561 | CATCATATAG TCCTACATCC TTTGTCATAA ATTAATTGAC CATAGGTGTG TGGGTTTATA |
| 10621 | TCTGGGCTCT CTATTCTGTT CCTTTGATCT ATGTGTCTGT TTTTATGCCA GCACCATGCT |
| 10681 | GTTTTGATTA CTATAGCTTT GTAGTATCAT CTGAAGTCAG GAAACATGAT TCCTCCAGCT |
| 10741 | TTGTTCTTCT TTCTCAAGAT TGTTTTGTCT ATTCAGAGTT TTATGTTCCT ATGCAGATTT |
| 10801 | TATTTTTATT TTTATTTTAT TTTTATTTTT TTTATTTTCC CACTGTACGG CAAGGGGGTC |
| 10861 | AGGTTATCCT TACATGTATA CATTACAATT ACAGTTTTTC CCCCACCCTT TCTTCTGTTG |
| 10921 | CAACATGAGT ATCTAGACAA AGTTCTCAAT GCTATTCAGC AGGATCTCCT TGTAAATCTA |
| 10981 | TTCTAAGTTG TGTCTGATAA GCCCAAGCTC CCGATCCCTC CCACTCCCTC CCCCTCCCAT |
| 11041 | CAGGCAGCCA CAAGTCTCTT CTCCAAGTCC ATGATTTTCT TTTCTGAGGA GATGTTCATT |
| 11101 | TGTGCTGGAT ATTAGATTCC AGTTATAAGG GATATCATAT GGTATTTGTC TTTGTCTTTC |
| 11161 | TGGCTCATTT CACTCAGGAT GAGATTCTCT AGTTCCATCC ATGTTGCTGC AAATGGCATT |
| 11221 | ATGTCATTCT TTTTTATGGC TGAGTAGTAT TCCATTGTGT ATATATACCA CCTCTTCTGA |
| 11281 | ATCCAATCCT CTGTCGATGG ACATTTGGGT TGTTTCCATG TCCTGGCTAT TGTGAATAGT |
| 11341 | GCTGCAATGA ACATGCGGGT GCACGTGTCT CTTTTAAGTA GAGCTTTGTC CGGATAGATG |
| 11401 | CCCAAGAGTG GGATTGCAGG GTCATATGGA AGTTCTATGT ATAGATTTCT AAGGTATCTC |
| 11461 | CAAACTGTCC TCCATAGTGG CTGTACCAGT TTACATTCCC AGCAGCAGTG CAGGAGGGTT |
| 11521 | CCCTTTTCTC CACAGCCCCT CCAGCACTTG TTATTTGTGG ATTTATTAAT GATGGCCATT |
| 11581 | CTGACTGGTG TGAGGTGGTA TCTCATGGTA GTTTTGATTT GCATTTCTCT TATAATCAGC |
| 11641 | GATGTTGAGC ATTTTTTCAT GTGTTTGCTG GCCATCTGTG TATCTTCTTT GGAGAAATGT |
| 11701 | CTATTCAGGT CTTTTGCCCA TTTTTCCATT GATTGATTGT TTTTTTTGCT GTTGAGTTGT |
| 11761 | ATAAGTTGCT TATATATTCT AGAGATTAAG CCCTTGTCAG TTGCACCTAT GCAGATTTTA |
| 11821 | AAACTATTTT CTCTAGTTCT ATGAAAAATA CCATTGGTAA TTTGATAGGG ATTGCCCTGA |
| 11881 | ATCTGTAGAT TGCCTTGGAT AGTATTGCCA TTTTAACAAT ACTGAATCTT CCAATTCGAG |
| 11941 | AGCACAGTGT ATCTTTCTTT CTGTGTCATC TTCAGTTCTT CTCATCTGCA TCTTATAGTT |
| 12001 | TTAGAAGTAC AGGTCTTTTG CCTCCTAAGG TGGGTTTTTT CCTAGGCATT TTATTCTTTT |
| 12061 | CAATGTGATA GTGAATGAAA TTGTTTCCTT AATTCTTTCT CTCTCTTTTT TAATGGCTTC |
| 12121 | ACCTGCAGCA TATGGAAGTC CCCAGGCTAG GGATCAAATC ACAGCTGCAG CTATGTCCAT |
| 12181 | GCCACTGCCT TGGCAACAGC AGATCTGAGC CACATCTGCC ACTTACACTG TAGCTTACAA |
| 12241 | TAATGCTGAA TCCTTAACCC ACTGCTAGAA CCTGAATCCT CACAGAAACA ATGTCGGGGT |
| 12301 | CCTTACCTCT CTGAGCCACA ATGGGAAATC TTCATTTTTC TTTCTGATAA TTTGTTGTTA |
| 12361 | GTGTATAGAA ATGAAACAGG TTTCAGCATA TTAATTCTTA TCCTGAAGTT TTACCCAATT |
| 12421 | CATTGATAAA CTCTAGTAGC TTTTTGGTGG TGTCTTTAGG ATTTTCTATG TATAGATTCA |
| 12481 | TGTTACCTGC AAACAGTGCC ATTATTACTT CCTTTTTTCC AAATTGGATT CCTTTTATTT |
| 12541 | CTTTTTCTTC TCTGCTGTGA CTAGGATTTC CAAAATCATG TTGAATAAAA GTAGCAAGAA |
| 12601 | TCAGCATCCT TGCTTTGTTC CTGACCTTAG AAGAAACACA TTCAGCATTT AACTGTCGAG |
| 12661 | TATGATGTTA GCTGTGGGCT TATCATATAT GGCATTTATT ATTTTGAGGT ATATTCCCTC |
| 12721 | TATACCCACT TTGTTGAGAA CTTTTTATCA TGAATGGATG TTAAACTTTG TCTAAAGCTT |
| 12781 | TTTCTGCATC TAGATAACCC TATTATTTTT CTTTTCTAAT TTGTTCATGT GGTGTATCAC |
| 12841 | ACTGATTTAT TTGCAGATGT GCATCCATTC ATGTATCCCA CTTGATCGTG GTGTGTAATC |
| 12901 | TTTTTAGTGT ATTAGTGAAT TTGGTTGCTA GTATTTTGTT TGAGGATTTT TGCATATACA |
| 12961 | TTCATCAGCG GTATTGGATT TTAAATCTTT TGTATGTGTC TTGTTTTGGT ATCAGGGTAT |
| 13021 | CCTCTAGGGT ATCCTCCTAG AATGAGTTCA GAAGGGTACA TTTCTTTGGG GAATATATTT |
| 13081 | GGTAGAATTC ACTTTTGAAG CTGTCTGGTC CTGTTCTTTT GTTTGTCGGG AAGTTCTTTT |
| 13141 | TAAATTATTA TTATTACTGA TTCAATTTCA TTACTGGTAA TTGGACCATT TATATTTTCT |
| 13201 | TTTTTTTCCT GGTTCAATCT TGGGAGATTG TATGTTTTAA AAATTTGTCC AGTTCTTCTA |
| 13261 | GGTTGTTCAT TTTATTGGAA TGTAATTGTT TGTTTATCTT TTTTTTTGCA TTTTCTAGGG |
| 13321 | CCGCACCCAT GGCATATGGA AGTTCCCAGG CTAGGGGTCT AATCGGAACT GTAGCCACTG |
| 13381 | GCCTACCCCA GAGCCACAGC AACGTGGGAT CTGAGCCGCA TCTTCGACCT ATACCACAGC |
| 13441 | TCACAACAAT GCGGGATCCT TAACCCACTG AGCAAGGCCA GGGATTGAAC CTGCAACCTC |
| 13501 | ATGGTTCCTA GTTGGATTAG TTAACCACTG AGCCACGACG GGAACTCCAA TGGTATGTAA |
| 13561 | TTGTTTATAG TGATCTCTTA TGAGTCTTTA TTTTTCTGTA GTAATCATAA CTTCTCTTAT |
| 13621 | TTCATTTTGA TCTTATTGAC TTGAGCCCTC TGTTTTTTTC TTAGTGACTC TAGCTAAAGG |
| 13681 | TTTATCAATT TTGTTCATTT TTTTCAAGGA TCTGGCTCTT AATTTCATTC AACTTTTCTA |
| 13741 | TTTATTTTAG TCTCTATTTC ATTTACTTCT GTTCAGATTT TTATGATTTC TTTCTTTCTA |
| 13801 | CTAAGTTCAG TTTTGGTTTG TTCTTTTCTA TTTCCTTTAA GTGTAAGGTT ATGTTGTTTA |
| 13861 | TTTGAGATTT TTGTTTCTTG AGGAAACAGG CTTGCATATT TGTAAACTTC CCTCTTAGAA |
| 13921 | TAGTTTTTCT TAAGTTCCAT AGTTTTTTTT TTTTATTTTG TGGTTTTTAT TTTTCCATTA |
| 13981 | TAGTTCATTT ACAGTGTTCT GCCAATTCCT ACTATATAGC AAAGTGACCC AGTCATATAT |
| 14041 | ATATGTATAT ATGTATATAT ACACATACAT ATACACATTA TCCTCCATCA TGTTCCATCA |
| 14101 | CAAGTGACTG GATACAGTTC CCTGTGCTAT ATAGCAGGAT CTCATTGCTT ATCCACTCCA |
| 14161 | AATGTAATAG TTTGCATCTA TTAACCCCAG ATGTCCCATA GATTTGGAAT TGTGTTTTTG |
| 14221 | TTTTCATTCG TATTCAGGTT TTTTTTAATT TCCTCTTTGA TTTCTTCAGT AATCCATTTG |
| 14281 | TTGCTTAGTA ATATATTGTT TAGCCTCTGC GTGTTTGTGG TTTGTTGCAA TTTTCTTCTT |
| 14341 | GTAGTTGATT TCTAGTCTCT TTGTGTTGTA GTTGGAAAAG ATGTATGATA TGATTTCAAC |
| 14401 | TTTCCTAAAT TTACCAAGGC TTGTTTTGTG GCCTAGCATG TGATATATCC TGAAGAATGT |
| 14461 | TCCATGTGCA CATGAAAAAA ATGAATATTC TGCTGCTTTC AAATGGAATG CTCTCTCTAT |
| 14521 | TTCAATTATG TCCATCTCTA ATGTTTTGGG AACATGTTCT TTTGCTACCT CATTTTGCCT |
| 14581 | AATTTGCTGT TTTGGGTTCT AAATATCTGG TAGGTTGGTT ACATTTTCCA ACCTTGGACA |
| 14641 | AATAACCTTT TGTTGAAACA TCCTGTGCTT CCCAGCAGCA CACTCCTCTC TGGTCACCAG |
| 14701 | AGCTATATGT TCCAGGGGTG CCCCCCTATG CTGACTTTGT GAGAACTTCT TTTGCAGTTG |
| 14761 | GCTGACTACT GTAGGTGGTC TTGTAGGCAT GGCTGGCCCC CAGTCTGGTT GTTTGCAAGA |
| 14821 | AGCTGCCTTG TACAAAGGCT GCCAGTCACT TGTTGGTGGG ACTGGGTCAT GGGGTGGCTG |
| 14881 | GCTATAGAGA CCAGGGTTGT CTCAGGGGTA GTGCTGTCTC ATTTGTGGGT TTAGCCACGT |
| 14941 | TTTGCAGTGG GTGATTGTGG TTCCAGGGTT CCTAGATCTA GTGTCAGCTT GTGGGTACTG |
| 15001 | GGGTCCCCAG CTGCAGGGCC TAGGAGCTTC AGAGCTAGAG CTAACCTCCT GGTGGGTAGA |
| 15061 | CTGTGTCCTG ACAAGGCAGG TTGTAGTGTT ACAGTGATCC TGGGGCTAGT ATCTATCCAC |
| 15121 | TGGGGGGTAA GACTTGTCCC AGGGCTAGCA CCAGCTCTCT GGTGGGTAGA TCTAGGTCCT |
| 15181 | GGAGGTTCTG GCTGCAGGGC CAGGGATCCA GGAGCTGGTG TTGACTGGTT GGTGGACAGG |
| 15241 | GCCAAGGCCC AGAGTGTCCC CAGGCTAGAT CTACTTCAGT GATGGGTGGA TCTAGGTCCT |
| 15301 | GTATTTCTGG CTACAGGGCT CTGGGATCCC AGAGTTGGTA TGTCAGTCAA CTGACATACA |
| 15361 | GGGCTGGAGG CAGAGAGTCC TGAGGCTGGT GCCTGCCCAC TGGTGGGTGG AGCTGGGATT |
| 15421 | CAGGGTCTCT GACTGAAGTG CCCTGGGGAT CCCTGGGCTA GTGCTGGCCC ACTGGTGTGT |
| 15481 | GTTTGGTTGG GTCCTGGCCA TTCTGGTAGA CAGGGCCATA TTCCCATATT CCAGGGTGGC |
| 15541 | TGTAGGCTCA GGGAATCTCA AGGCAACCTA CTGCTGGTTA GAGGAGTGTG TGGGGAGGTG |
| 15601 | CTATGTCCCT GTCCAGTTTG TTGCTTGGCA TGAAGCATCC CAGTACTGGT GCCAACAGGC |
| 15661 | TAATTAGTGG GTCTGGGTCC TGGTGCTAAT AAGCTAGAGG GAAGATTCAA AAATGACATT |
| 15721 | TTTTTAACAC CAGTGTCCTT GTGGTAAAAT GAACTCCCCA GAATGGCTAC CACCAGTGTC |
| 15781 | TATGTCCCCA TGGTGAATTC TAATTGCTCC TGTCTCTTGA AGTGGCTCTC CAAGATCAAC |
| 15841 | AGGTGGGTCT GATCTAAGCT CCTTTCAAAT TACTGCTTCT GCCCTGGGTC CCAGAACATG |
| 15901 | TGAGATTTTG TGTGTCCTTT AAGAGTGGAG TCTCTATTTC CCACTGCTCT CTGGTTCTCC |
| 15961 | CCAAAGTAAG CCCTGCTGGC TTTCAAAACT TCTGGGAGCT TGCCTTCTTG GTATAGGACT |
| 16021 | CCTGGGCTAG GGAGTCTAAT GTTTGGCTTA GACCCCTTAC TGCTTGGGAA GAATCTCTGC |
| 16081 | AACTGTAATG AATTATCTTC CTATTTGTGG GTTGCTGAGG ATATGGTCTT AACTGTTCTG |
| 16141 | TGTTCTACCC CTCCTATCCA TCTTGTTGTG GTTCCTTCTT TATATCTTTA GTTGTAGAAA |
| 16201 | AGTTTTTCTT ATCAACAGTT GCTCTGTAAA TTGTAACTTG GGTGTACACC TAGTAGGAGG |
| 16261 | TGAGCTCAGG GTCTTCCTAC TCTGCCATCT TGGCCATGTC CTCTAAACAT TTTGGTGTAT |
| 16321 | TTCACTGCAA CCTTTTTAAA AATCTCAAAA GTGAGCTGTG ATTGGCTAGT CTTGTGGATA |
| 16381 | ATCTCTAGCA TTTGATGCTA ATCATATTTA TACAAATACT TTGTTGAAAA GTGATGCCTT |
| 16441 | TTTAACTATT ATTAAAAAAC GTATTGACAT AACTATTGCT ATTATACTGA AAAGAAAGAC |
| 16501 | CTTAGAGAAA ATAGCATAAG AGCAAAACCA TTAAACATGG AGACATCTAG TCATAGGGTG |
| 16561 | GAAATTTTAT GTGGTGCATA TCCCCTAACC AGTGGCTTTA CACCAGGCAC ATCCTAACTA |
| 16621 | AGATCTGCTC CCAAGTGTCT TCCCTGATGC TTTAAATTGT GTTACATGGA AACTATCCTT |
| 16681 | TGATGAAGAA ATGCAACCTT TTAAAATACA ACATTGAAAC TTTTGTGCTT TAATTTTGCT |
| 16741 | TTTCAACATT TTTTCTTTTT AAAAGAAGAA ATTTATTTGT TTTTTTAAAT TTTAATGGCC |
| 16801 | ACGGCATATG GAAGTTCTCA GGCCAGGGAT AGAATTCAAG CCACAGGTGC GACCCATGCC |
| 16861 | ACAACTGCTG CAACACCAGA TCCTTTAACC CACTGCACCA GGCCAGGGAT TGAAGCCTTG |
| 16921 | CCTTACTGAC AATCTGAGCC ACTTCAGTCA GATAAAGAAA TTTCTTCATT AAGCAGAGTA |
| 16981 | TTCACATGGT TTAAACTTCA AAATATTAAA GTGTAAACTC TTTCCCCACC ACTGTCCCCA |
| 17041 | GCTCACCAAC TCTACTTACC ACAGACAACT GATGTGGTTA GGGTATTTAA ATAGTAAATC |
| 17101 | CAAGAAAATA TAAACAAATC CGTATATATA GGTTTCACCC CATTTTATTA TCCTAATGTT |
| 17161 | GCATATCATA TAAACTATAC TGTCCCTTGG GTATTCACTT AGTAAAATAT TTTGATCATA |
| 17221 | ATTTCCTATC AGTATTTAAA GAGCTTTCTG AAATTATTTC TGTATAACAT TTCTTTTCTC |
| 17281 | ATCATCTATT ATGTGCATTT ATTTATATTT TAACTTCTTT TATTAGATGA AATTATCTTC |
| 17341 | TGCTTCAGCT TTTTTTTTTT TTTAAGAACA CACAGTTGGG TTTTTTAAGG TTAATACCAC |
| 17401 | CTTTGTTTTC TAAGTCATTA AATTTGTTTT TCTATTAATT CACTTCTGAT TCTTTGAAGT |
| 17461 | TTGATTTCTT TTTAGCTTTT AACTTCTTGA GTTGTATGCT TAATTAATTT TGATTCTTTC |
| 17521 | CTATTTATTA ATATACATAT TTGAAGCTAT AGGTTTTCCA CTGAGTATAC CAGTAGCTAT |
| 17581 | ATCGTATAAT TGATGAACTG ATCCTCTGTG AGTCTGGGAC ATAAACGTCC TATGACTGTT |
| 17641 | ATGTGGTAGC TGTGAATTGC TCTTTTTAGA TTATAAAGTT CTCATCTTTT ATAGTTGAAC |
| 17701 | AATTTTTGTC CTGAATCAAA TTTGTTGGAT ATTAATATCA CATCTATTGC TTTATTTATT |
| 17761 | TTCTATTCTC ACTTTTAACC TCTGTGAATA ATTTCACTCT AGGTGCCTCA CTTTTTTCAT |
| 17821 | AATAGAATTG GGATTTATTT TTAAAAGGAC TCTGATTAAG TAATTTTCTT TTTCTGATAT |
| 17881 | GGGAGATATA TTTGACCTTA ACTTAGTCAC ATTATGCATT GTTCTCTTGT CATGTTATGT |
| 17941 | ATACATAACA TTTATTGTCA TTATGGTACA ACTAAAAACA TATTTCACTC TGTGACCTTT |
| 18001 | ATGGGGACTC AGCATTTGTT TAGGAATGTG GAAGTATATT TGTATATCTG ATAATTTCCT |
| 18061 | TCCAAATTTA AAAAGGTTTG TATATTTTCA TATTAACATA TTTCATATTA ATTAGCATGA |
| 18121 | ATTTCAGCTG CATTAAAAGG AAAACCACCT GAGTGGTAAA GAAAAAGTTT TTTTTTCTCT |
| 18181 | TTTTTTTTTT TTTTTTTTTA ATGGCCACAT CTGTGGCATG TGAAGTTCCC AGGCTAGGGG |
| 18241 | KCGAATAGGA GCTACAGCTG CCAGCTTGCA CCACAGCCAC AACAATGCCA GAGCCAAGCC |
| 18301 | TCATCTGCGA CCTATACCAC AACTCATGGC AATGCTGGTT CCTTAACCCC CTGAGTGAGG |
| 18361 | CCTGGGGTCA AACCCACATC CTCATGGATA CTAACCGGCT TTGTTACCGC TGAGCCATGA |
| 18421 | GGGAAACTCC CTTTTTCTCA TTGAAAATAA GTCAAATAGA TAAGCAGCTT AAGGCTGTTT |
| 18481 | GGGTGATTCT GTGGTCCAGT AATTATCAAA TCCTACTGGA CAAGAATAGA GAATGTGCAA |
| 18541 | ATGAGGGAAC GTGTTGGTGA GATCAGGCTC TGCCCACTGA GCTATCCTCT GTCATGGGCC |
| 18601 | CTGTGCTGTT CTCAGAGCTG TACTTCCTAG GGCATTGTTC TCATTTCAAT TCTGAGTTCA |
| 18661 | GTGTGGAGAG TATACGTGTG TGGGGGCTGC ACGCTTTTCA CAACCCACTT TCTGCTGATA |
| 18721 | CTGATTTAGG GATCCTTGGA TTGCTTTACA GTTGAGTCAT CATTAACTAG TGTCACTTGC |
| 18781 | CTTCAAAGTC AGCAAAATAA TTGTCTCCAA ACTAGTAGGC TTCTAGTGTA TTTGCTTTAA |
| 18841 | TCCAATGCCA TGTGAAAGTA ACATGGTCAA AGAATAAGTT ATATACCTTG ACCTACCCTG |
| 18901 | TGACCAGGCT CTTCCTCTTA ATTTATTGAC CACTGCCTTA AGGTCATTTG AAACCATGGG |
| 18961 | TTTGGGAGGA AGGCAAGGCC TAAATCCCGT CTTTGTTGGA AGGCTCACTG TCCTTGTCTT |
| 19021 | TAGAGCATCA TTTTTTTTTA AACTGGGGTA CAGTTTATTT ACAGTGTTGT GTCAATTTCT |
| 19081 | GCTGTACAGC ATAGTGACCC AGTCATACAC ATACATACAT TCTTTTTCTC ATACTATCTT |
| 19141 | CAATTTTATT TTGTGCTAAG TCTGCCATTT TATCATCACC TCAGTTTGAA GGACAGGATA |
| 19201 | TTTAGAGTTT GTTTTTTTTT TCCCCCCAAT CCTGCAATTT CTAAATTATA AGACTCTCAA |
| 19261 | TTAGCCGTAT ATAACAGCTG CAGGCACAGG ATGTCTCCCT CACAAAATTG GTATTTTTCC |
| 19321 | TTCCATTTCT TCTTGCAGTT TGGCTATTTC TTGTCTGAGT TCATCTCTCT TTTTAAGTGT |
| 19381 | TAAAAAGGGC AAGGAGGATT CATGCTATGT CAACATTATG ATTTTTTCTT TTCTATACTT |
| 19441 | GATAAGAGTA TACTTTTCCC AAATGTCATC CAACTTTTCA GCATCAGTTT GGACATGGTT |
| 19501 | TTCTTTTCAA GGTGGTATTT CTCTAATGTC ACTTGAATAA CAAGACTCGT TAGTTCTCCA |
| 19561 | GGCTACAATA TCCTAGTCTG AGTATATTCT GCATGTTAAT TCTATTCAGC CACATCCATA |
| 19621 | ATTTAGGTTT TATTCCTGGA ACACCTCACT TTTTTTTTTT TTTTGGTCTT TTTATAGCCA |
| 19681 | TAACCATGGC ATATGGAGGT TCCCAGGCTA GGGGTCTAAT CTGAGCTTTA GCCACTGGCC |
| 19741 | CATGCCACAG CCACAGCCAT GCCACATCTG AGCCACATCT GTGACCTTTT CCACAGCTCA |
| 19801 | CAGAAACACC AGATCCCTAA CCCACTGAGT GAGGCCAGGG GTCAAACCTG TAACCTCATG |
| 19861 | GTTCCTAGTC AGATTCGTTT CCTCTGTACC ACGATGGGAA TTCCTAATAC CTCACTTATG |
| 19921 | ATAACACATT CTGAATTATT TAGGATTCTA TTATACTGCA TGTAATAGAA ATCCCAAATA |
| 19981 | GCAAAATTTG CAACTTAAGG CAGGTTCCTG TCTTTACAAA ATCATGTTTT CCTTTGCTAT |
| 20041 | ATGTGCACTT TGCTTTCCTC TGTGAATTCC CTTTTTTGTT ATATTTCTAT AGCTTTTGGA |
| 20101 | AACACTTTTA CTTATTTGGG GGGGCCTAGA TTTTTAACCC TCTCCTTGTT TTTCTAGAAA |
| 20161 | TAGAGTTTAT AATTTTATTT CTTCATTTAC TTGATACTTT CAAGAGATTT CCAGGAAAAA |
| 20221 | AATTATGGAA ATACTGTCTC TGTGCCTGCC AAGTTCAAAC TAAGAATTGT ATAATCTGTT |
| 20281 | TTAATTCTTA AGCATTTATA GATGACAAGG CTTTGTGTCT GATAGGGGCC AGCGAACTCA |
| 20341 | GTAAAGAGGG AAGATGAGAA AGATAATGGC AAGAATTTAT CCCTGAAGTG TAGTTTTGAC |
| 20401 | AAACCAGTCA CAAAGAGGTC TAAGAAATTT TGGTCACAAA GTTGTTTTGA ATCCCAGGCA |
| 20461 | TTTTATTTGC AATGATTGCA TATGTTCTGG AAAGGACATC TGAACCTAAG AAATAGTTCA |
| 20521 | TTTGCATTGT GTTATATTTT ACTAAGGTCT GAGAAATAAT CTTGAGATGA GAATGAACTC |
| 20581 | TACTTCTTCA GAGTCTGGAA GGAATAAATT ATGAAAATGT ATTAATGCTT CTTTAAACCA |
| 20641 | TATTGTATAT TTATCTATTA CTAAACAAAA AGAAGTAGCT CTATTTATTT ATTTATTTAT |
| 20701 | TTATTTATTT ATGTCTTTTG TCTCTTTAGG GCCACACCTG TGGCATATGG AGGTTCCCAG |
| 20761 | GCTAGAGGTC CAATTGGAGA TGTAGCAGCC AGCCTATGCC AGAGCCACCG CAACACGGGA |
| 20821 | TCTGAGCCAC GTCTGTGACT TACACCACAG CTCACAGCAA CGCCTGATCC TCAACCCACT |
| 20881 | GAGCGAGGCC AGGGATCGAA CCCATGTCCT CATGGATGCT AGTTGGGTTC ATTAACTGCT |
| 20941 | GAGCCATGAT GGGAACTCCA AATTAATTAT TTCTTATATT TGTTCTTCAT ATATTCATTT |
| 21001 | CTATAGAAAG AAATAAATAC AGATTCAGTT AATGATGGCA GGTAAAAGCT TAACTTATTA |
| 21061 | ATCAAAGGAG TTAATCCAGG CACAAAAATT CAATTCATGG CTCTCTGTTA AAATTTAGGT |
| 21121 | ATAGGTTTAG CAGGAAGAAA AGGTTAGTAG ATGCAGACTA TTACATTTAG AATGGATGGA |
| 21181 | CAATGAAGTC CTACTATACA GCACAGGGAA CTATATCCAA TCTCTTGGGA TAGAATATGA |
| 21241 | TGGAAGACAA AATCAGAACA AGAGAGTATA TATATATGTG TGTGTGTGTG TGTGTGTGTG |
| 21301 | TGTGTGTGTG TGTGTGTGTG ACTGGGTCAC CCTGCGGCAC AGCAGAAATT GGCAGAACAT |
| 21361 | TGTAAATCAA CTATACTTTA ATAGGAAAAA TACTTTTAAG GGCTAAATTT CCAATATTCT |
| 21421 | AACCATGTAC ACAGAGTAAA TGTCATAAGG ATGCCAGTCT GTGTAGAGAT TGATGTGTTA |
| 21481 | CTAGCAGATT CATGAAATAA AGGCTGAGGA TGTAGTCCCC AAGTCACTTC TGAGTGGAAG |
| 21541 | AATTTCTCCT TTGTCCTGGA CTCAAATATT TTAGGATAAA GGAAAAAAGA AGATATTTAT |
| 21601 | AGAAGGGACT TGTTTTCAAG TACTTGACAA AATTTCACCA TTAAAGAGAA ATTTGTGGGA |
| 21661 | GTTCCCATCG TGGCTCAGTG GAAACAAATC CAACTAGGAA CCATGAGGTT GTGGGTTTGA |
| 21721 | TCCCTGGCCT CACTCAGTGG GTTAAGGATC CGGTGTTGCC GTGAGCTGTG GTGTAGGTTG |
| 21781 | CAGACACGGT TCTGATCCTG CGTTGCTGTG GCTGTGGCTG TGGTGTAGGC CAGCAGCAAA |
| 21841 | CAGCTCTGAT TAGACCCCTA GCCTGGAAAC CTCCATATGC CACAGGTGCA GCCCTAAAAA |
| 21901 | GACAAAAAAA GAGAAAAGAC AAAAAAAAAA AAAAAAAAAA AAAAAAAAAA AAAAAAAAAN |
| 21961 | NNNNNNNNNN NNNNNNNNNN NNNNNNNNNN NNNNNNNNNN NNNNNNNNNN NNNNNNNNNN |
| 22021 | NNNNNNNNNN NNNNNNNNNN NNNNNNNNNN NNNNNNNNNA GAACCACCAG AGGTATTTAT |
| 22081 | TTGTTTTTGC CTTTTTTCAC TGACTGTTTT TTGTTTGTTT GTTTGAGACT GATCTAGAAG |
| 22141 | ACTAGAGATT ACAAGAAATA TGGATTTGGC TCACTCTAAG AAACTGCTTT CATTCCAAGG |
| 22201 | TTTGGGTCTA TCCAAAAGTG GAATAGAATC ATATGAATAC TAGTTTATGA GTATTTAGTG |
| 22261 | AGAGGAATTT CAAGCTCAAA TAATGATTCA GCAAGATTAA ATTAAGGAGG GAATTTTCCT |
| 22321 | TGTGGCTGAG TGGGTTAAGG ACCCAATGTT GTCTCTGTGA GGATGTAGGT TCCATCCTGG |
| 22381 | GCTTTGCTCA TTAGGTTAAG GATCTGGCAT TGCTGCAGCT CAGACCCAGT GCTGCCCTGG |
| 22441 | TTGTGGCTTA GGCCAAAGCT GCAGCTCCAA TTCAATCTCT GGCCTGGGAA CCTCCATGTG |
| 22501 | CTACAAGGTG CGGCCTTAAA AGGAAAAAAA AAAAAATTAA ATCAAGGACT CAAGAGTCTT |
| 22561 | TCATTATTTG TGTTGTGGAA GCTATATTTG TTTTAAAGTC TTAGTTGTGT TTAGAAAGCA |
| 22621 | AGATGTTCTT CAACTCAAAT TTGGGAGGGA ACTTGTTTCA TACATTTTTA ATGGATAAGT |
| 22681 | GGCAAAATTT TCATGCTGAG GTGATCTATA GTGTTGTAAT GCAGAATATA GTCAGATCTT |
| 22741 | GAACATTTTA GGAAGTTGGT GAGGGCCAAT TGTGTATCTG TGCCATGCTG ATAAGAATGT |
| 22801 | CAAGGGATCA CAAGAATTCG TGTTATTTGA CAGCAGTCAT CTTTAAAAGG CATTTGAGAA |
| 22861 | AGTCCAATTT CAAATGCATT TCCTTTCTTT AAAAGATAAA TTGAAGAAAA TAAGTCTTTA |
| 22921 | TTTCCCAAGT AAATTGAATT GCCTCTCAGT CTGTTAAAAG AAACTCTTAC CTTGATGATT |
| 22981 | GCGCTCTTAA CCTGGCAAAG ATTGTCTTTA AAATCTGAGC TCCATGTCTT CTGCTTTATT |
| 23041 | TCTGGTGTGC CTTTGACTCC Agattacagt aaatggagga cTGAGTATAG GGCTAAAAAG |
| | GAT\|T-------sgSL27---- |
| 23101 | TAGAGAGAAT GGATGCATAT TATCTGTGGT CTCCAATGTG ATGAATGAAG TAGGCAAATA |
| 23161 | CTCAAAGGAA AGAGAAAGCA TGCTCCAAGA ATTATGGGTT CCAGAAGGCA AAGTCCCAGA |
| 23221 | ATTGTCTCCA GGGAAGGACA GGGAGGTCTA gaatcggcta agcccactgt AGGCAGAAAA |
| | sgSL26----------C\|TGT |
| 23281 | ACCAAGAGGC ATGAATGGCT TCCCTTTCTC ACTTTTCACT CTCTGGCTTA CTCCTATCAT |
| 23341 | 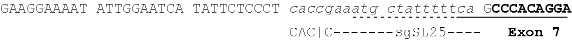 |
| 23401 | 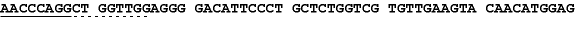 |
| 23461 | ACACGTGGGG CACCGTCTGT GATTCTGACT TCTCTCTGGA GGCGGCCAGC GTGCTGTGCA |
| 23521 | GGGAACTACA GTGCGGCACT GTGGTTTCCC TCCTGGGGGG AGCTCACTTT GGAGAAGGAA |
| 23581 | GTGGACAGAT CTGGGCTGAA GAATTCCAGT GTGAGGGGCA CGAGTCCCAC CTTTCACTCT |
| 23641 | GCCCAGTAGC ACCCCGCCCT GACGGGACAT GTAGCCACAG CAGGGACGTC GGCGTAGTCT |
| 23701 | GCTCAAGTGA GACCCAGGGA ATGTGTTCAC TTTGTTccca tgccatgaag agggtaGGGT |
| | ----sgSL28-------GG\|GTA |
| 23761 | 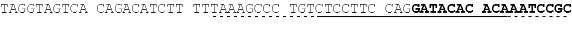 |
| 23821 | 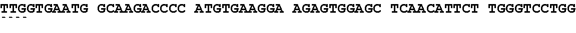 |
| 23881 | GGGTCCCTCT GCAACTCTCA CTGGGACATG GAAGATGCCC ATGTTTTATG CCAGCAGCTT |
| 23941 | AAATGTGGAG TTGCCCTTTC TATCCCGGGA GGAGCACCTT TTGGGAAAGG AAGTGAGCAG |
| 24001 | GTCTGGAGGC ACATGTTTCA CTGCACTGGG ACTGAGAAGC ACATGGGAGA TTGTTCCGTC |
| 24061 | ACTGCTCTGG GCGCATCACT CTGTTCTTCA GGGCAAGTGG CCTCTGTAAT CTGCTCAGGT |
| 24121 | AAGAGAATAA GGGCAGCCAG TGATGAGCCA CTCATGACGG TGCCTTAAGA GTGGGTGTAC |
| 24181 | CTAGGAGTTC CCATTGTGGC TCAGTGGTAA CAAACTCGAC TGGTATCCAT GAGGGTATGG |
| 24241 | GTTTGATCCC TGGCCTTGCT CAATGGGTTA AGGATCCAGC ATTGCTGTGA GCTGTGGTAT |
| 24301 | AGGTTGCAGA CTCTGCTCAG GTCCCATGTT GCTGTGATTG TGGTGTAGGC TGACTGTTGC |
| 24361 | AGCTTCAATT TGACCCCTAG CCCGGGAATT TCCATAGGCC ACACGTGCAG CACTAAGGAA |
| 24421 | GGAAAAAAAA AAAAAAAAAA AAAAGAGTGG GTGTGCCTAT AGTGAAGAAC AGATGTAAAA |
| 24481 | GGGAAGTGAA AGGGATTCCC CCATTCTGAG GGATTGTGAG AAGTGTGCCA GAATATTAAC |
| 24541 | TTCATTTGAC TTGTTACAGG GAAAGTAAAC TTGACTTTCA CGGACCTCCT AGTTACCTGG |
| 24601 | TGCTTACTAT ATGTCTTCTC AGAGTACCTG ATTCATTCCC AGCCTGGTTG ACCCATCCCC |
| 24661 | CTATCTCTAT GGCTATGTTT ATCCAGAGCA CATCTATCTA ACACTCCAGC TGATCTTCCT |
| 24721 | GACACAGCTG TGGCAACCCT GGATCCTTTA ACCAACTGTG CCAGGCTGGA GATCAAACCT |
| 24781 | AAGCCTCTGC AGCAACCCAA GCTGCTGCAG TCAGATTTTT AACCCCCTGT GCCACTGTGG |
| 24841 | GTATCTCCGA TATTTTGTAT CTTCTGTGAC TGAGTGGTTT GCTGTTTGCA GGGAACCAGA |
| 24901 | GTCAGACACT ATCCCCGTGC AATTCATCAT CCTCGGACCC ATCAAGCTCT ATTATTTCAG |
| 24961 | AAGAAAATGG TGTTGCCTGC ATAGGTGAGA ATCAGTGACC AACCTATGAA AATGATCTCA |
| 25021 | ATCCTCTGAA ATGCATTTTA TTCATGTTTT ATTTCCTCTT TGCAGGGAGT GGTCAACTTC |
| 25081 | GCCTGGTCGA TGGAGGTGGT CGTTGTGCTG GGAGAGTAGA GGTCTATCAT GAGGGCTCCT |
| 25141 | GGGGCACCAT CTGTGATGAC AGCTGGGACC TGAATGATGC CCATGTGGTG TGCAAACAGC |
| 25201 | TGAGCTGTGG ATGGGCCATT AATGCCACTG GTTCTGCTCA TTTTGGGGAA GGAACAGGGC |
| 25261 | CCATTTGGCT GGATGAGATA AACTGTAATG GAAAAGAATC TCATATTTGG CAATGCCACT |
| 25321 | CACATGGTTG GGGGCGGCAC AATTGCAGGC ATAAGGAGGA TGCAGGAGTC ATCTGCTCGG |
| 25381 | GTAAGTTCTG CACATCACTT CGGGTTACAA TGATTTAAGA AACAACTAAG GTGGGGCAAA |
| 25441 | GGGTAGTGAG GCATATCCAT CAGAGCAAAT TCCTTGAAAT ACGGACTCAG AGGAAACCAT |
| 25501 | TGTGAGATTG AGGTTCCCAG AGGTGTGGAT TTAATGAATT AGTGTTACCT CATGTACAAG |
| 25561 | GTAGTATACT ACCAGAAAGA TAAAAATTCA GAAGCGAGTT TGCAGCAAAA CTCATAGGGA |
| 25621 | GAACTTCTTT TATAAATAAT ATGAAGCTGG ATATTTAGTG CACCACCTGA TGACCACTTT |
| 25681 | ATTAATAAAT AAAGAGTTCC TGTTGTGGCG CAGCGGAAAT GAATCCGACA AATAATCATG |
| 25741 | AGTTTGCGGG TTTGATCCCT GACCTCGCTC AGTGGGTTGG GGATCTGGTG TTGCCATGAG |
| 25801 | CTGTGGTGTA GGTCGCAGAT GCTGCTTGGA TCCCGCTTTG CTGTGGCTGT GGTATAGTCT |
| 25861 | TGTGGCTACA GCTCCGATTT GACCGCTAGC CTGGGAACCT CCATATGCTG CGGGGGTGGC |
| 25921 | CCTCAAAAGC AAAATAAATA AATAAGTAAA TAAATAAGTA GTTTAAAAAG GACAAGAAGA |
| 25981 | AATATATTTG GTATTATATT CTACAGAGAC AAAGATAATC ACCATGCCCG ATTGATTTTT |
| 26041 | CAAGGCATAT AAATGAGACG TCATGGGAGC AAAAATGGTC ATAATACAAT GCCCTTGTTT |
| 26101 | TGTGTACATG GTAAGATTTT AGAAAGCATT GTGAAGTAGA AAAGTGTACT CAGTTATAAT |
| 26161 | ATATTGGAGA AAACAGTACT ATGAGAAGTA AAAAAATCTA CATGCCGGAA TTTATTTTTT |
| 26221 | TAATGTCTCT TTAGAGTCGC ACATGCGGCA TGTGGAGGTT CCCAGGCTAG GGGTCGAATC |
| 26281 | AGAGCTATAG CCACTGGCTT ATGGCACAGC CACAACAACG CTAGATCTGA GCCACATCAG |
| 26341 | AGACCTATAC TATAGCTCAT GGCAATGCCA GATCCTTAAC CTACTGAGCC AAGCCATGGG |
| 26401 | TCAAATCCAG GTCCTCATGG ATCCTAGGCA AATTCATTTC TGCTGAGCCA CGAAGGGAAC |
| 26461 | TCCTCAGAAG TGATTTTGAT GTTACTTTCT TTTCATGACA AATCTGGTAA AGTACATACA |
| 26521 | CATAGAAACT GAAGTGTCAG AAAGGGAAAT ATTTCATTTT AAGGTAATGT ATACAAAACA |
| 26581 | GTGGTTTTAC CATCTGAGTA TCTTGCTAAA TTTTAACTAT CAAGGACAAT TGCCAAAAAA |
| 26641 | AAAAAAAAAA GAGAGAGAGA GAGAACAGAA TAGGGTTATG AAGCTAAAAT CACAGGGTTA |
| 26701 | TGAAGCTAAA ATCACAGTAA TTTAGGGAGA AAAAAATCCA AAGCATGTAA TTGATAAAAG |
| 26761 | GCTCTGAGCC TTTGTTTGAG ATTTAGAATT CAACTTGGAA ATACCGGTGG TATTTTAAAG |
| 26821 | CAGTCCATAA GTATAAAATC CAAGGCTAAA AAGCCAGAAG GTATTTGTAG AACAAATATA |
| 26881 | TTTTAATAAG CTCTACCAAG TCATCCAGAA GCTACTAAAG AATTACTGGT CACTGACATA |
| 26941 | GTGTACCTGT TTTCAAGGCC ATTCTTACAT CAGAATAAAG GGAGAGCACC CTCTGAATCT |
| 27001 | TCAGAAAAGA TGTGAAAGTG CTAATTCTCT ATTTCATCCC AGAGTTCATG TCTCTCAGAC |
| 27061 | TGATCAGTGA AAACAGCAGA GAGACCTGTG CAGGGCGCCT GGAAGTTTTT TACAACGGAG |
| 27121 | CTTGGGGCAG CGTTGGCAGG AATAGCATGT CTCCAGCCAC AGTGGGGGTG GTATGCAGGC |
| 27181 | AGCTGGGCTG TGCAGACAGA GGGGACATCA GCCCTGCATC TTCAGACAAG ACAGTGTCCA |
| 27241 | GGCACATGTG GGTGGACAAT GTTCAGTGTC CTAAAGGACC TGACACCCTA TGGCAGTGCC |
| 27301 | CATCATCTCC ATGGAAGAAG AGACTGGCCA GCCCCTCAGA GGAGACATGG ATCACATGTG |
| 27361 | CCAGTGAGTA TCCATTCTTT AGCGCCACTG TTATCTTCTG ATCTACCTAA GCAGAAGTGT |
| 27421 | TATAACCTTT AGATAATCCC TATTCTACCT GGATGATGAG ATTCATTCTC TTTAATTTGG |
| 27481 | TGTGCAGGTA TTCAGGATCA GTGATCATTT TCCCAAAGAC CATCATGCTC TGATGGTCTT |
| 27541 | CTCAAAAGTT CTAATCAGTT GCTTCCTCCG TGAACAGTTG AGGAGCAGAG AATATGTAAT |
| 27601 | TCAGAATTTG ACTATTGAAT CATCCCATTT TTCTTTCACA TAGTCTTTTG TTGCACTGAG |
| 27661 | TATAAGGAGA GAAGCAGTCA GAAAGATCAA TCCTGAATTA TTTCTCCATT CTACATCTGT |
| 27721 | TTTAAATTTC AAAAAAAATT GTTATAGGTG ATTTACAATG TCTGTCAATT TCTGCTCTAC |
| 27781 | AGCAAAGTGA CCCAGTTATT TACATATACA TTCTTTTTCT CATATTTTTA AACCGGGAGA |
| 27841 | TTTCTATCCA CCTGGCAGTT TGAGGGAATT TAACATTATG CATTTATGTT AACTTTATTC |
| 27901 | ACCTGATGTT TTCTAAGTCA TACTGAGATT CTTATGTCCA GGATGGAATA CACCTGGTTT |
| 27961 | GCTGGAAAGA CATGTGCTTT CATAAAGATG AATTTTGGAA AAAATATAAA ATTTAAAAGT |
| 28021 | CCCATTAAAT AAGCAAAGTT TTAAGAGATT TCAAAAAAAA TTTCATCTCT CTCTTTTCCT |
| 28081 | CTTTGACCTC TTGGGCACGT TCATCTTCTC AAATATGATC TTGGTGTTTC TGACTTTTCA |
| 28141 | GACAAAATAA GACTTCAAGA AGGAAACACT AATTGTTCTG GACGTGTGGA GATCTGGTAC |
| 28201 | GGAGGTTCCT GGGGCACTGT GTGTGACGAC TCCTGGGACC TTGAAGATGC TCAGGTGGTG |
| 28261 | TGCCGACAGC TGGGCTGTGG CTCAGCTTTG GAGGCAGGAA AAGAGGCCGC ATTTGGCCAG |
| 28321 | GGGACTGGGC CCATATGGCT CAATGAAGTG AAGTGCAAGG GGAATGAAAC CTCCTTGTGG |
| 28381 | GATTGTCCTG CCAGATCCTG GGGCCACAGT GACTGTGGAC ACAAGGAGGA TGCTGCTGTG |
| 28441 | ACGTGCTCAG GTGAGGGCAG AGAGTCTGGA TTGAGCTTGG AAGCTCTGGC AGCAAAGAGA |
| 28501 | GGGTGGGCGG TGACCTGCAT TGGGTAAAGA TTGGAAGGTC CAGCCTAAGG ATCTGGTGGT |
| 28561 | GGGGGGAGAC ATGATGTTTC AGTCTGAAGA ATGATGAAAA CCTGTGTTGT TACGCATGGG |
| 28621 | CCTTCACCGA GGAAAGGAAC ATAACTTACA TGTATCCTCC TGCAGAGGGA GGAAGAACTA |
| 28681 | GGGGATTCTA GTTTTGTGTG GGAAGGAGCA GTTTACTTGG TTCAGGAGGC ACTAAAGGCT |
| 28741 | CAGATAGGAA ACAGAGATCT GTTCCATTCT TACTCCCAGA ACTGATTCTC TTCTCTTTTC |
| 28801 | TCCTACAGAA ATTGCAAAGA GCCGAGAATC CCTACATGCC ACAGGTATAT AAAAAAGTTT |
| 28861 | AAGAACATGG GACCCATTGT CTGCATTTTG TGGAATCCCT CTTATTAAGA CATTCTGGGT |
| 28921 | CAGAAGTTCT GAGGATTTGA CATTTACTTC AGCTATCTGT TATCTTACCC AAGAGAGGGA |
| 28981 | TGGTAACTAG GAACCCAGGT CTTTTAGCTA AGACATTATC ACCTCTTGTG ATGTTTACTT |
| 29041 | GTTCTCAGGT CGCTCATCTT TTGTTGCACT TGCAATCTTT GGGGTCATTC TGTTGGCCTG |
| 29101 | TCTCATCGCA TTCCTCATTT GGACTCAGAA GCGAAGACAG AGGCAGCGGC TCTCAGGTCT |
| 29161 | GAACAAAATT ACGGTCTCTC TAATGTTTCT ATGGGATAAG AAGCCTCTCT GGATAATAAA |
| 29221 | ACAAAAAAAT TACATTCAAG TATCAGTTGG CCAGAAAGAG GGAACCTAGA AGAGGTTTAA |
| 29281 | GCAGTTTCTC CGAAACAGGG AACAAGAATT CAGAGAAGAA AAGGCACATT GGCTGTACTG |
| 29341 | ATGATACCTG CACTCGCTAT GTATGTTTAA TGGGGGACAG TAGAGAATTG ATAGTTTAGA |
| 29401 | AGGAGTATGC TTATATGGTT CTGGATGAAT CCTGTATCCC CCCAAACATT TATTTTCTCT |
| 29461 | TACTATATAC TTATTACTAA TTTAACTCTT CTGTCAAGCC GTGTGCTAGG TTCTGAAGAT |
| 29521 | GGTTCAGACT TGGATACTCA AGTGCTTTTG TTTTCATGGA ATTTCCAGTT TAGTGGAAGA |
| 29581 | GATAAATATG TAAACAAATA AATTGCAATG TTTTATTATA CATTCGTGTG AATAAGGAAC |
| 29641 | AAAGGAGGCA CAGAGAATAA AGTAATTACT GAAAGGGGAA GGGGAGTATC AGAGACTTCT |
| 29701 | AAGTTTGGAG GCAGATTTTG AAGACAGAAA TCAAAGTACT GGGTAAGATG CATTTCAGGA |
| 29761 | AAGAAGAAAA ATATGTACAC GTGTAGAGAA GCTTAAAAGA GGGCACATTT GTTGTTTTGG |
| 29821 | AGGGGAGTAC AAGTTGAGTT AAAGAGAGAA GTTTCTGTTA AGGCTGAAGA ATAGGGAAGA |
| 29881 | TACACGTAGC GATGCTCTGT GTTGCATGAT AAGAAGAGTC GGAGTTATTA AAGAGTATGA |
| 29941 | GATAGGGGAG TGAGATAGGC AGGCAGGTCC TTAGAAAGTT CTGTTTGGAA ATGGGATGTC |
| 30001 | GGAGGGGTTG AAAGAGAACC ATATATTGAC AAGGAGAGCA TTTTGAAGTA GTTGTGATGA |
| 30061 | AAGATAAAAT GGACTTTATA GTGAGAATGG CTGGGAAAGG ATAGATTTTA TACAAATCTC |
| 30121 | CAATGAATTA CAGAAGAATG CTACCTGTCT TTGGGGAAGA AACAGGGTTA TCCGATGGCA |
| 30181 | TCCTGTTGCG TTTGAGTTCG TGACATCATG AGGGAAAGGC TTGGCAGCGT TTACTCGGTA |
| 30241 | CTGTGTGGTA ACTTATATGG AAAAAAATAT GAGAAGGAAT GAGTGTGTGT ATAACTAATT |
| 30301 | TACTTAGCTG TATGCCTGAA ATTAATACAA TTTTATAAGT CAACTCTACT CCAATAAAAC |
| 30361 | AAACAAATAA ATAAATAATT TTAACTACCT GAACAAAAAA AAAAGAATGG ACTGGAGACA |
| 30421 | AGTCAAAAGT ATGGATGATG ACTACGTTAT GCTTGCACTG CTGGGGAAAA GCACACATAG |
| 30481 | GGAGGGAACG TTTTATTATG ACCCAGTCCC TAACCTATGA CCTCTGTTAT CAGTTTTCTC |
| 30541 | AGGAGGAGAG AATTCTGTCC ATCAAATTCA ATACCGGGAG ATGAATTCTT GCCTGAAAGC |
| 30601 | AGATGAAACG GATATGCTAA ATCCCTCAGG TCCGTGGGTT CTTTGAGGGC CTGTAGCCCT |
| 30661 | GGGGTTCAGA TCAGCAGCTG CAGTTGAGGT TGAGGCATGC TACTTTGCAC AGCAGTAGAA |
| 30721 | AGAAATCTCA ACTGTAATAG GAAGCTTGGG ATGCATATGA GGAAGAAAGG CAAGAATGAA |
| 30781 | CCACAAATTA TTCTTAGGGA AGATAAAAAT TGCAGTCATG GGGAGACCTC TGGCTGAGAG |
| 30841 | GGCCGTGATT ATTTCTGACA GAGGGATTAT GGAGTAGAAT ATGATGGCTT GGACCTTTTT |
| 30901 | TCACTAAAAC AAGTCAGTCT TCTCAAAGGT AGTTTAGCTT TTCATATATC TTTCTCAGTT |
| 30961 | TCTTCCATTC CCATTTCCTG CCATTTTCCT TTCTCTAACT TTTATTTATT ATATTTTTTC |
| 31021 | CTAAAAGTTT AAATTTTCTA TATCTTTATC CCTTCAGAAG CCATCCCTAG TCACAGGACT |
| 31081 | AGTTTTATTT CCCATTATGT AATGCTTCTT TCTCTGTCTG TTGACTTCTA TTTAGAACCA |
| 31141 | GTGCACTAAA TCTGCCTCTA GGAACATACC TCTGCTAGGT TGCAAGAAAT ATCCCATTCC |
| 31201 | CCACTCACTC TGTGAAGACT CAATGCTTCT CAATATTCCT TACCTCCTGA GAGGGACTTG |
| 31261 | CCTCACTTCT TTAATCCAAG GGACTCGATT TTTGCCAAAA CTAAGTCAGG AAAACCTACA |
| 31321 | TAAGACATAG GAAAGACTTG CTGTGCTTCT TAAACCCCAC TGTTTGTTTT CCTAATTGTG |
| 31381 | AACAGTATTT TTAAAGTTCA AAGAGCTTCT AAGGCACTTG AGGGGAGATC TGATTTATTT |
| 31441 | CCCAGTAATT ATTTTATTCC TTTCAGAAAA TTCCAATGAA TAAGATGGTT TTAATGATGT |
| 31501 | GGGACTAATT TTTGTGTCTA AATCTCTTCC TATTTCTGGA TGAAAAAAAG GAGACCACTC |
| 31561 | TGAAGTACAA TGAAAAGGAA AATGGGAATT ATAACCTGGT GAGGTGAGTA AAAAGAATTT |
| 31621 | ATTCATCATT GCTGAAAACA GGTACATTCC TTTTGAAAGT TGGGAACTCC TCTGGTATTA |
| 31681 | GAAAAAAAAA AAAGAACGTA TATACACATA TATTTCCATG TCTATGTTTA TGTTTGTAAA |
| 31741 | TCCATATTCA GAATATGCAA CAACTTTTTA TAACTATGAC TTCAGTCCAT CTTTTAGTTA |
| 31801 | CATATATATT CTAAACAACA ACTATTGCTA AGAGAAGCTG GGTAAGTAAA TGTGAATAAA |
| 31861 | TCTTCTAAAG ATATTACAGG AAGTTCCTGC TGCGGCTCAG TGGGTTAAGG ACTTGATGTC |
| 31921 | TTTGTGAAGA TGAGGGCTCG AGCCCTGGCC TCACTCAGTG AGTTAAGGAT CTAGCATTGC |
| 31981 | TGTAAGCTGC AGCGTAGGTT GCAGATGGGG CTCAGATCCA GTGTTGCTGT GGCTGTGGCC |
| 32041 | TCAGTTGCAG CTCTGATTCA ACCCTTAGGC GAGGAACTTC CATATGCAGC AAATGTGGCC |
| 32101 | ATTAAAAAAA AACAAAAAAC ATTATAGGAG TCATTTCATA AAAGAGATAA GACGTTTCTA |
| 32161 | TAGTTATATA GTGCATACTC TGGTAAAGAT AGTATAGGAT ACTATAGGAA TATAGAAAGC |
| 32221 | TTGCCTATGA AAATTTGGGA AGATTGTGGA AAAGACATCT CAAAATATGG CATAGAAAAG |
| 32281 | AATCATATCT TTGAGGAACA GTAAGTTTTT CATTCAAAAC CGTGTATTGA ACATACTTAT |
| 32341 | GGTGACAAAT GGTGTCTTGA GTACTAAAAA TTCAGTGATA AAAGATGCTC TTGACAAAGA |
| 32401 | CATGGCTGTT GAATAGAAGG TCTCACTGTC AATGTGTGGG AATTATGGAC AGCCTATGTG |
| 32461 | GACACAGGGA ATAGATGAGA CTCTAGGCTG GAAGGCTGCA TTGAGCCCAG TAATGAATGG |
| 32521 | TCCTGTCTGA TATATTTCAT GCTCATATTT TATTTTAGGG ACTATTGGGG AGGTGGTGGG |
| 32581 | CTTTGGAAGA TTAAGCTGAG GCAAGACACA ATCAGATTGC CTTTTATAAT TTACTTTCAG |
| 32641 | GAGGAAAATC TAACTAAAGA AAAAAAGTGA ATAAGGCAAG AAACATAAGT TATACATCAA |
| 32701 | AAAGAAAAGG TAGTGGAGTT CCTGTTGTGG CTCAGTGGTT AATGAACCCT GCTAGGAACC |
| 32761 | ATGAGGTTGT GGGTTCGATC CCTGGCCTTG CTCAGTGGGT TAAGGATCCA GCGATGCCAT |
| 32821 | GAGTTGTGGT GTAGGTCGCA GACCGTGGCT TGGGTCCCGC ATTGCTGTGG CTATGGTGTT |
| 32881 | GGCTGGCAGC TGCAGACAGC TCTGATTA |

1. A genetically edited swine, the swine comprising an edited genome wherein the edit results in the deletion of SRCR5 domain from the CD163 protein produced by the swine.

2. The genetically edited swine of claim 1 wherein all of the other domains of the CD163 protein are present and their amino acid sequences are unaltered.

3. The genetically edited swine of claim 1 wherein the CD163 protein produced by the genetically edited swine remains substantially functional.

4. The genetically edited swine of claim 1 wherein the CD163 protein lacks the following amino acid sequence:
(SEQ ID NO: 2)
HRKPRLVGGDIPCSGRVEVQHGDTWGTVCDSDFSLEAASVLCRELQCGTV
VSLLGGAHFGEGSGQIWAEEFQCEGHESHLSLCPVAPRPDGTCSHSRDVG
VVCS.

5. The genetically edited swine of claim 4 wherein the CD163 protein produced by the genetically edited swine has no further changes to the wild type amino acid sequence.

6. The genetically edited swine of claim 1 which is homozygous or biallelic for the genome edit that results in the deletion of the SRCR5 domain from the CD163 protein produced by the animal.

7. The genetically edited swine of claim 1 wherein all cells of the animal comprise the edited genome.

8. The genetically edited swine of claim 1 wherein the genome of the swine is edited such that the sequence which codes for SRCR5 is absent from the mature mRNA produced from the edited CD163 gene.

9. The genetically edited swine of claim 1 wherein the swine comprises an edited genome in which exon 7 of the CD163 gene has been deleted.

10. The genetically edited swine of claim 1 wherein the splice acceptor site located at the 5′ of exon 7 of the CD163 gene is inactivated.

11. The genetically edited swine of claim 1 wherein exons 1 to 6 and 8 to 16 of the CD163 gene are unaltered relative to the wild type sequence.

12. The genetically edited swine of claim 11 wherein exon 7 and portions of introns 6 and 7, which flank exon 7, are deleted from the CD163 gene, but there are no other alterations in the remaining regions of the CD163 gene.

13. The genetically edited swine of claim 1 wherein the edited genome is edited such that the splice site donor sequence in intron 6 and the splice site acceptor site in intron 7 are unaltered and remain functional.

14. The genetically edited swine of claim 1 wherein the genome is edited such that at least a portion of the region of the CD163 gene extending from position 10466 to 23782 with reference to SEQ ID NO:1, is deleted.

15. The genetically edited swine of claim 1 wherein the genome is edited such that regions from positions 1 to position 10465 and from position 23783 to position 32908, with reference to SEQ ID NO:1, are unaltered.

16. The genetically edited swine of claim 1 wherein the genome is edited such that exon 7 is deleted along with up to 5000 bases, suitably up to 2000 bases, suitably up to 1000 bases, suitably up to 500 bases, suitably up to 300 bases or suitably up to 100 bases extending 5′ of the 5′ end of exon 7.

17. The genetically edited swine of claim 1 wherein the genome is edited such that exon 7 is deleted along with up to 75 bases extending 3′ of the 3′ end of exon 7.

18. The genetically edited swine of claim 1 wherein the genome is edited such that the edited genome comprises a deletion of the region extending from:
a) approximately position 23060 to approximately position 23760, for example from position 23065 to position 23753, with reference to SEQ ID NO:1;
b) approximately position 23260 to approximately position 23760, for example from position 23268 to position 23753, with reference to SEQ ID NO:1; or
c) approximately position 23370 to approximately position 23760, for example from position 23374 to position 23753, with reference to SEQ ID NO:1.

19. The genetically edited swine of claim 1 wherein the edited genome comprises an inserted sequence.

20. The genetically edited swine of claim 1 wherein the genome is edited such that the region extending from position 23378 to position 23416, with reference to SEQ ID NO:1, is edited such that the splice acceptor site in intron 6 is inactivated.

21. The genetically edited swine of claim 1 wherein the splice acceptor site in intron 6 is partially or entirely deleted, or its sequence altered in any other suitable way so that it is no longer functional.

22. The genetically edited swine of claim 20 wherein the splice acceptor site is edited to alter the sequence from AATGCTATTTTTCAGCCCACAGGAAACCCAGG (SEQ ID NO: 3) to AATGCTATTTTTCgGCCatggGGAAACCCAGG (SEQ ID NO: 4), wherein the sequence changes are shown in lower case.

23. The genetically edited swine of claim 1 wherein the genetically edited swine has improved tolerance or resistance to PRRSV infection compared to a wild type swine, preferably wherein the animal is resistant to PRRS infection.

24. A genetically edited swine cell or embryo, wherein the edit results in the deletion of SRCR5 domain from the CD163 protein that can be produced by the swine cell or embryo.

25. A method of producing a genetically edited swine, the method comprising the steps of:
a) providing a swine cell;
b) editing the genome of the cell to create a genome modification which results in the deletion of SRCR5 from the CD163 protein; and
c) generating an animal from said cell.

26. The method of claim 25 wherein the genome modification that results in deletion of SRCR5 from the CD163 protein is deletion of exon 7 from the CD163 gene or the inactivation of the splice acceptor site in intron 6 of the CD163 gene.

27. The method of claim 25 wherein in step a) the swine cell is a somatic cell, a gamete, a germ cell, a gametocyte, a stem cell (e.g. a totipotent stem cell or pluripotent stem cell) or a zygote.

28. The method of claim 25 wherein in step a) the swine cell is a single cell zygote and step b) of the method is at least initiated in the zygote at the single cell stage.

29. The method of claim 25 wherein in step b) comprises:
introducing a site-specific nuclease to the cell, the site-specific nuclease targeting a suitable target sequence in the CD163 gene;
incubating said cell under suitable conditions for said site-specific nuclease to act upon the DNA at or near to said target sequence; and
thereby induce an editing event in the CD163 gene that results in deletion of SRCR5 from the CD163 protein.

30. The method of claim 29 wherein the editing event that results in deletion of SRCR5 from the CD163 protein is the deletion of exon 7 from the CD163 gene or the inactivation of the splice acceptor site in intron 6 of the CD163 gene.

31. The method of claim 29 wherein step b) comprises introducing site-specific nucleases to the cell which are targeted to target sites flanking exon 7 of the CD163 gene so as to induce double-stranded DNA cuts on either side of exon 7 and thereby cause its deletion.

32. The method of claim 31 wherein one target site is in intron 6 and the cutting site is 3′ of the splice donor site at the 3′ end of exon 6, and wherein another target site is in intron 7 and the cutting site is 5′ of the splice acceptor site at the 5′ of exon 8.

33. The method of claim 25 wherein step b) comprises introducing an upstream site-specific nuclease to the cell, the upstream site-specific nuclease targeting a target site upstream of exon 7 of the CD163, and introducing a downstream site-specific nuclease to the cell, the downstream site-specific nuclease targeting a target site downstream of exon 7 of the CD163.

34. The method of claim 29 wherein step b) comprises introducing a site-specific nuclease that targets the splice acceptor site in intron 6.

35. The method of claim 34 wherein the site-specific nuclease that targets the splice acceptor site in intron 6 creates a single double stranded cut at the desired cutting site to inactivate the splice acceptor site associated with exon 7 by non-homologous end joining (NHEJ) or by homology directed repair (HDR).

36. The method of claim 35 comprising providing an HDR template having following sequence: GAAGGAAAATATTGGAATCATATTCTCCCTCACCGAAATGCTATTTTTCgGCCatggGGAA ACCCAGGCTGGTTGGAGGGGACATTCCCTGCTCTGGTC (SEQ ID NO:16), wherein lower case letters show the changes made compared to the unaltered sequence.

37. (canceled)

38. The method of claim 25 comprising the steps of:
providing a swine zygote;
introducing a site-specific nuclease to the zygote, the site-specific nuclease targeting a suitable target sequence in the CD163 gene;
incubating said zygote under suitable conditions for said site-specific nuclease to act upon the DNA at or near to said target sequence and thereby induce an editing event in the CD163 gene that results in deletion of SRCR5 from the CD163 protein; and
generating an animal from said genetically edited zygote.

39-44. (canceled)
